(19) United States
(12) Patent Application Publication
(10) Pub. No.: US 2026/0258504 A1
(43) Pub. Date: Sep. 3, 2026

(54) METHYLATION MARKER FOR CANCER

(71) Applicant: Methylomics B.V., Rijswijk (NL)

(72) Inventors: Joachim Bastiaan BOERS, Rotterdam (NL); Ruben Gerdo BOERS, Rotterdam (NL); Joost Henk GRIBNAU, Rotterdam (NL); Wilhelmus Gregorius Vincentius QUINT, (deceased) (NL); Willem Frederik Johannes VAN IJCKEN, Gouda (NL); Henricus Arno Marie VAN DEN MUNCKHOF, Nootdorp (NL); Kirkyra Chrisnelle Deftera TADEMA, Vlaardingen (NL); Liselotte Jacoba Maria MARTENS, Zwijndrecht (NL); Bobby Adrianus DRIESSEN, Zoetermeer (NL)

(21) Appl. No.: 19/141,942

(22) PCT Filed: Dec. 22, 2023
(86) PCT No.: PCT/NL2023/050686

§ 371 Date: Jun. 20, 2025

(30) Foreign Application Priority Data
Dec. 22, 2022 (EP) 22216119.2

(51) Int. Cl. C12Q 1/6886

(52) U.S. Cl. CPC C12Q 1/6886; C12Q 2600/106; C12Q 2600/112; C12Q 2600/118; C12Q 2600/154

(57) ABSTRACT

The invention relates to methods of identifying a gynaecological or ano-uro-genital tissue or cell sample or a tonsil, skin, blood, plasma, serum, saliva, serous fluid, urine or stool sample as indicative of cancer or as indicative of a predisposition to cancer, comprising determining the methylation status of one or more genes and/or promoter regions thereof and/or one or more chromosomal regions. The invention further relates to methods of classifying an individual as suffering from or being at risk of suffering from a gynaecological, ano-uro-genital, head and neck or cutaneous cancer, methods for determining methylation status of the one or more genes and/or promoter regions thereof and/or one or more chromosomal regions, method of treating an individual suffering from or being at risk of suffering from a gynaecological, ano-uro-genital, head and neck or cutaneous cancer and kits of parts comprising means for the detection of DNA methylation of one or more genes and/or promoter regions thereof and/or one or more chromosomal regions.

### FIELD OF THE INVENTION

The invention relates to the field of testing for cancer and for susceptibility to cancer, in particular gynaecological, ano-uro-genital, head and neck or cutaneous cancer. In particular, the invention relates to novel methylation markers, in particular multi-cancer methylation markers, and uses thereof.

### BACKGROUND OF THE INVENTION

The epidemiology of gynaecological and ano-uro-genital cancers is undergoing change. Cervical cancers are a major public health concern and although other anogenital cancers such as those of the vulva, vagina, penis, bladder and anus are relatively uncommon cancers, they are increasing in incidence. Screening programmes and the HPV vaccine are important in the prevention and early diagnosis of anogenital cancers. Infections with high-risk human papillomaviruses (hrHPV) can cause cervical cancer, as well as other anogenital cancers and head-and-neck cancers. However, most HPV infections remain asymptomatic. In addition, not all HPV-induced precancerous lesions progress to cancer.

Although human papillomavirus (HPV) infection has been found in most of the cervical cancer cases, and other cancer including gynaecological or ano-uro-genital cancer additional genetic and epigenetic changes are required for disease progression. Previously, it was thought that only genetic mutation plays a key role in cervical cancer development. But recent advances in the biology of cervical cancer revealed that epigenetic alteration is common in for instance cervical carcinogenesis and metastasis.

DNA methylation during carcinogenesis has been widely studied in recent years, as well as the development of cancer-specific methylation markers. Due to the tissue-specific pattern, DNA methylation is used to discriminate the tissue of origin for cancers. However, pan-cancer (also referred to as multi-cancer) DNA methylation biomarkers have not been extensively examined. Pan-cancer markers provide an attractive early general screening for cancer or the risk thereof. A number of studies on pan-cancer DNA methylation markers have been performed, but not for gynaocological and ano-uro-genital cancers. For instance, Liu et al. (Front. Cell Dev. Biol. 2021; https://doi.org/10.3389/fcell.2021.649168) identified differentially methylated sites and regions across 10 cancer types including breast cancer, colorectal cancer, head and neck carcinoma, kidney clear cell carcinoma, kidney papillary cell carcinoma, liver cancer, lung adenocarcinoma, lung squamous cell carcinoma, prostate cancer, and thyroid cancer; Yang et al. (Brief Bioinform. 2017 Sep. 1; 18(5):761-773. doi: 10.1093/bib/bbw063.) investigate pan-cancer wide DNA methylation patterns across 15 different cancer types including bladder urothelial carcinoma (BLCA), breast invasive carcinoma (BRCA), colon adenocarcinoma (COAD), esophageal carcinoma (ESCA), head and neck squamous cell carcinoma (HNSC), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), pancreatic adenocarcinoma (PAAD), prostate adenocarcinoma (PRAD), rectum adenocarcinoma (READ), thyroid carcinoma (T HCA) and uterine corpus endometrial carcinoma (UCEC); Saghafinia et al. (Cell Rep. 2018; 25(4):1066-1080.e8. doi: 10.1016/j.celrep.2018.09.082.) investigated hyper- and hypo-methylation markers in 24 cancer types. Li et al. (Evid Based Complement Alternat Med. 2022 Aug. 18; 2022:1150390. doi: 10.1155/2022/1150390) describes that expression of ARID3C is linked to hepatocellular carcinoma (HCC), while high expression thereof is associated with prolonged survival, and that CpG methylation in ARID family members is associated with HCC prognosis. WO 2016/109782 describes methylation markers for colorectal cancer. One of the markers (SEQ ID NO:12) is located on chromosome 8 (chr8:96161265-96161346).

Effective screening programs are critical to reduce the incidence and mortality. High-risk HPV (hr-HPV) testing as a primary screening tool discloses high sensitivity for HPV induced cancers but suboptimal specificity, and of course exclude non-HPV induced cancers. Adequate triage tests to reduce unnecessary colposcopy referrals and overdiagnosis and overtreatment are crucial for both HPV induced and non-HPV induced cancers. Hence, there remains a need in the art for DNA methylation-based markers, including pan-cancer markers, for both HPV and non-HPV induced gynaecological and ano-uro-genital cancers as triage test that have both high sensitivity and specificity.

### SUMMARY OF THE INVENTION

It is an object of the present invention to provide novel methylation markers that can differentiate between normal cells and samples and cells and samples predisposed to cancer. It is a further object of the invention to provide multi-cancer markers.

The invention therefore provides a method of identifying a gynaecological or ano-uro-genital tissue or cell sample or a tonsil, skin, blood, plasma, serum, saliva, serous fluid, urine or stool sample as indicative of cancer or as indicative of a predisposition to cancer, comprising determining the methylation status of one or more genes and/or promoter regions thereof and/or one or more chromosomal regions selected from table 1.

In a further aspect, the invention provides a method comprising determining methylation status of one or more genes and/or promoter regions thereof and/or one or more chromosomal regions selected from table 1 in a test sample. Said test sample is preferably a gynaecological or ano-uro-genital tissue or cell sample or a tonsil, skin, blood, plasma, serum, saliva, serous fluid, urine or stool sample. The cancer is preferably a gynaecological, ano-uro-genital, head and neck or cutaneous cancer, more preferably a HPV induced cancer or non-HPV induced gynaecological, ano-uro-genital, head and neck or cutaneous cancer.

In a further aspect, the invention provides a method for classifying an individual as suffering from or being at risk of suffering from a gynaecological, ano-uro-genital, head and neck or cutaneous cancer, the method comprising determining DNA methylation of one or more genes and/or promoter regions thereof and/or one or more chromosomal regions selected from table 1 in a sample of said individual, and classifying said individual based on said DNA methylation.

In a further aspect, the invention provides a method for determining methylation status of one or more genes and/or promoter regions thereof and/or one or more chromosomal regions selected from table 1 in a sample of an individual, the method preferably comprising contacting said sample with means for detecting methylation status of said one or more genes and/or promoter regions thereof and/or one or more chromosomal regions selected from table 1.

In a further aspect, the invention provides a method of treating an individual suffering from or being at risk of suffering from a gynaecological, ano-uro-genital, head and neck or cutaneous cancer, the method comprising:
classifying an individual according to a method of the invention;
identifying an individual that is suffering from or at risk of suffering from a gynaecological, ano-uro-genital, head and neck or cutaneous cancer; and
referring of the individual to the appropriate medical specialist, the method further preferably comprising:
identifying the gynaecological, ano-uro-genital, head and neck or cutaneous cancer; and
regular examining of the status and/or monitoring of the cancer or precancerous lesion; and/or
providing treatment of the cancer or precancerous lesion the identified individual, such as local resection or cryosurgery, laser therapy, hysterectomy, salpingo-oophorectomy, penectomy, transurethral resection, cystectomy, prostatectomy, polypectomy, colectomy, proctectomy, pelvic exenteration, laryngectomy, thyroidectomy, radiation therapy, chemotherapy, hormone therapy, immune therapy and combinations thereof. As will appreciated by the skilled person, the type of treatment depends on the cancer or precancerous lesion identified.

In a further aspect, the invention provides a use of one or more genes and/or promoter regions thereof and/or one or more chromosomal regions selected from table 1 as a multi-cancer marker for a gynaecological, ano-uro-genital, head and neck or cutaneous cancer and precancerous lesions thereof.

In a further aspect, the invention provides a use of one or more genes and/or promoter regions thereof and/or one or more chromosomal regions selected from table 1 as a multi-cancer marker for detection of cancer or a precancerous lesion in a gynaecological or ano-uro-genital tissue or cell sample or a tonsil, skin, blood, plasma, serum, saliva, serous fluid, urine or stool sample.

In a further aspect, the invention provides a use of one or more genes and/or promoter regions thereof and/or one or more chromosomal regions selected from table 1 as a multi-cancer marker for identifying a gynaecological or ano-uro-genital tissue or cell sample or a tonsil, skin, blood, plasma, serum, saliva, serous fluid, urine or stool sample as indicative of cancer or as indicative of a predisposition to cancer.

In a further aspect, the invention provides a use of one or more primers and/or probes specific for the detection of DNA methylation in one or more genes and/or promoter regions thereof and/or one or more chromosomal regions selected from table 1 as a multi-cancer marker for gynaecological, ano-uro-genital, head and neck or cutaneous cancer and precancerous lesions thereof.

In a further aspect, the invention provides a kit of parts comprising means for the detection of DNA methylation of one or more genes and/or promoter regions thereof and/or one or more chromosomal regions selected from table 1.

In a further aspect, the invention provides a use of a kit of parts according to the invention for classifying an individual as suffering from or being at risk of suffering from a gynaecological, ano-uro-genital, head and neck or cutaneous cancer.

In preferred embodiments, the methylation status is determined or detected using methylation specific PCR. In preferred embodiments, the methylation status is determined or detected using methylation specific primers and/or probes. In preferred embodiments, hypermethylation of one or more genes and/or promoter regions thereof and/or one or more chromosomal regions selected from table 1 indicates a sample of an individual suffering from gynaecological, ano-uro-genital, head and neck or cutaneous cancer or a sample of an individual that is predisposed to such cancer.

In a further aspect, the invention provides a method for classifying an individual as suffering from or being at risk of suffering from cervical cancer, vulvar cancer, colorectal cancer, anal cancer, or precancerous lesion thereof, the method comprising determining DNA methylation of chromosomal region METloc009 as indicated in table 1A, GLIS3 gene or a promoter region thereof and/or FRMD4B gene or a promoter region thereof in a sample of said individual, and classifying said individual based on said DNA methylation.

In a further aspect, the invention provides a method for determining methylation status of chromosomal region METloc009 as indicated in table 1A, GLIS3 gene or a promoter region thereof and/or FRMD4B gene or a promoter region thereof in a sample of an individual, the method preferably comprising contacting said sample with means for detecting methylation status of said METloc009, GLIS3 gene or a promoter region thereof and/or FRMD4B gene or a promoter region thereof.

In a further aspect, the invention provides a method for detecting cervical cancer, vulvar cancer, colorectal cancer or anal cancer or precancerous lesion thereof, the method comprising detecting hypermethylation in chromosomal region METloc009 as indicated in table 1A, GLIS3 gene or a promoter region thereof and/or FRMD4B gene or a promoter region thereof.

In one embodiment, a method is provided for detecting cervical cancer, vulvar cancer, colorectal cancer or anal cancer or precancerous lesion thereof, the method comprising detecting hypermethylation in chromosomal region METloc009 as indicated in table 1A, whereby hypermethylation indicates the presence of cervical cancer, vulvar cancer, colorectal cancer and/or anal cancer or precancerous lesion thereof, in particular cervical cancer, vulvar cancer, colorectal cancer and/or anal cancer.

In one embodiment, a method is provided for detecting vulvar cancer or precancerous lesion thereof, the method comprising detecting hypermethylation in GLIS3 gene or a promoter region thereof as indicated in table 1A, whereby hypermethylation indicates the presence of vulvar cancer or precancerous lesion thereof, in particular vulvar cancer.

In one embodiment, a method is provided for detecting cervical adenocarcinoma, the method comprising detecting hypermethylation in FRMD4B gene or a promoter region thereof as indicated in table 1A, whereby hypermethylation indicates the presence of cervical adenocarcinoma.

In a further aspect, the invention provides a method of treating an individual suffering from or being at risk of suffering from cervical cancer or vulvar cancer or precancerous lesion thereof, the method comprising:
classifying an individual with a method according to the invention;
identifying an individual that is suffering from or at risk of suffering from cervical cancer or vulvar cancer or precancerous lesion thereof; and
referring of the individual to the appropriate medical specialist, the method further preferably comprising:
identifying the cervical cancer or vulvar cancer or precancerous lesion thereof; and
regular examining of the status and/or monitoring of the cancer or precancerous lesion; and/or
providing treatment of the cancer or precancerous lesion the identified individual, such as local resection or cryosurgery, laser therapy, hysterectomy, radiation therapy, chemotherapy, hormone therapy, immune therapy and combinations thereof.

In preferred embodiments of these methods detection of hypermethylation in chromosomal region METloc009 as indicated in table 1A and/or GLIS3 gene or a promoter region thereof indicates the presence of vulvar cancer or a precancerous lesion, in particular vulvar cancer.

In further preferred embodiments of these methods, detection of hypermethylation in chromosomal region METloc009 as indicated in table 1A indicates the presence of cervical cancer or a precancerous lesion, in particular cervical cancer.

In further preferred embodiments of these methods, detection of hypermethylation in chromosomal region METloc009 as indicated in table 1A indicates the presence of colorectal cancer or anal cancer or a precancerous lesion, in particular colorecta cancer or anal cancer.

In further preferred embodiments of these methods, detection of hypermethylation in FRMD4B gene or a promoter region thereof indicates the presence of cervical adenocarcinoma.

In a further aspect, the invention provides a use of one or more primers and/or probes specific for the detection of DNA methylation in chromosomal region METloc009 as indicated in table 1A, GLIS3 gene or a promoter region thereof and/or FRMD4B gene or a promoter region thereof as a marker for detecting cervical cancer or vulvar cancer or precancerous lesion thereof or colorectal or anal cancer or precancerous lesion thereof.

In a further aspect, the invention provides a kit of parts comprising means for the detection of DNA methylation of chromosomal region METloc009 as indicated in table 1A, GLIS3 gene or a promoter region thereof and/or FRMD4B gene or a promoter region thereof, preferably wherein said means comprise one or more primers and/or probes specific for said chromosomal region METloc009 as indicated in table 1A, GLIS3 gene or a promoter region thereof and/or FRMD4B gene or a promoter region thereof, or to a bisulfite converted sequence thereof.

In a further aspect, the invention provides a use of a kit of parts according to the invention for classifying an individual as suffering from or being at risk of suffering from cervical cancer or vulvar cancer or precancerous lesion thereof or colorectal or anal cancer or precancerous lesion thereof.

### DETAILED DESCRIPTION

The present inventors identified and validated novel methylation markers that can differentiate between normal cells and samples and cancerous cells and samples or cells and samples predisposed to cancer. In particular, it was found that hypermethylation of the relevant genes, promoter regions or chromosomal regions is indicative for a cancer and for a risk of developing cancer.

The novel methylation markers were identified by methylated DNA sequencing (MeD-seq), a method to detect DNA methylation profiles genome wide. Methylation dependent restriction enzyme LpnPI was used. Digestion by LpnPI generates small DNA fragments containing methylated CpG sites which are isolated and prepared for Next Generation Sequencing. In contrast to other methods, like bisulfite sequencing and array platforms, it omits the need for bisulfite conversion and enables genome wide profiling of DNA methylation in an unbiased manner. Because the DNA damaging bisulfite conversion is not needed, MeD-seq requires low amounts of DNA input compared to other methods. This makes MeD-seq very suitable in combination with other applications such as Laser Capture Microdissection (LCM), liquid biopsy samples (e.g. cell free DNA) and small populations of FACS sorted cell types.

The markers identified in table 1 are multi-cancer markers. In particular, they have been demonstrated to be hypermethylated in a variety of gynaecological and ano-uro-genital cancers, as well as in head and neck and cutaneous cancer. As demonstrated in the Examples herein, all multi-cancer markers, and in particular the markers Metloc001, Metloc002, ARID3C and ARL5C identified herein, show a high sensitivity and specificity. No DNA methylation markers have been previously identified that are useful for the wide variety of gynaecological, ano-uro-genital, head and neck or cutaneous cancers that can be detected with the present multi-markers. Such markers are in particular useful as a first screening for the presence of cancer, in particular gynaecological and ano-uro-genital cancers and head and neck and cutaneous cancer. Hence, the markers can be used in first screening of samples obtained using non-invasive methods, such as cervical scrapes or swabs, urine, stool, saliva and blood, including self-obtained samples. The markers of the invention can be used prior to the use of cancer-specific markers and, as such, lead to a large reduction in the use of such cancer-specific markers. With the markers of the invention it has become possible to assess the presence or absence in cancer or a risk thereof in a sample. A major advantage thereof is that only positively tested individuals need to be subjected to cancer-specific diagnosis, using for instance cancer-specific markers. This substantially reduces the number of gynaecological and ano-uro-genital cancers and head and neck and cutaneous cancer-specific tests that have to be performed, as well as the total number of tests.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The use of the alternative (e.g., “or”) should be understood to mean either one, both, or any combination thereof of the alternatives.

The percentage of identity of a nucleic acid sequence, such as a primer or probe, or the term “% sequence identity”, is defined herein as the percentage of residues of the full length of a nucleic acid sequence that is identical with the residues in a reference nucleic acid sequence after aligning the two sequences and introducing gaps, if necessary, to achieve the maximum percent identity. Methods and computer programs for the alignment are well known in the art, for example “Align 2”. Programs for determining nucleotide sequence identity are also well known in the art, for example, the BESTFIT, FASTA and GAP programs. These programs are readily utilized with the default parameters recommended by the manufacturer.

As used herein, the term “gynaecological cancer” refers to any cancer that originates in a woman's reproductive organs. In preferred embodiments, the gynaecological cancers include cervical cancer, endometrial cancer, vulvar cancer and ovarian cancer. In preferred embodiments, the gynaecological cancer is a carcinoma. Examples of such carcinomas are adenocarcinomas, squamous cell carcinomas, mucinous carcinomas, clear cell carcinomas, serous carcinomas and endometrioid carcinomas. A gynaecological cancer as used herein is preferably selected from the group consisting of cervical, vulvar, endometrial, ovarian, fallopian tube and vaginal cancer.

As used herein, the term “ano-uro-genital cancer” refers to anal cancer and any cancer that originates in the organs of the reproductive system and the urinary system. In preferred embodiments, the ano-uro-genital cancer is a carcinoma. Examples of such carcinomas are adenocarcinomas, squamous cell carcinomas, mucinous carcinomas, clear cell carcinomas, serous carcinomas and endometrioid carcinomas. An ano-uro-genital cancer or precancerous lesion as used herein is preferably selected from the group consisting of cervical, vulvar, endometrial, ovarian, fallopian tube, vaginal, colon, colorectal, anal, penile, bladder and prostate cancer and precancerous lesions, more preferably from the group consisting of cervical, vulvar, endometrial, ovarian, colon and colorectal cancer and precancerous lesions.

As used herein, the term “HPV induced cancer” refers to a cancer that is induced by high-risk human papilloma virus (hrHPV), such as HPV induced cervical, vulvar, vaginal, anal, head and neck (in particular tonsil), penile, and cutaneous cancer.

As used herein, the term “non-HPV induced cancer” refers to a cancer that is not induced by high-risk HPV, such as most or all occurrences of endometrial, ovarian, colon, colorectal, bladder, prostate, fallopian tube cancer.

Cancers defined herein as HPV induced also present as non-HPV induced cancers. The methylation markers of the present invention do not discriminate between, and are predictive for both HPV-induced and non-HPV induced cancers.

Cancer of the cervix is the fourth most common cancer in women world-wide. Cervical squamous cell carcinoma development is preceded by the formation of precancerous lesions, so-called cervical intraepithelial neoplasia (CIN), with three different grades. These range from mild dysplasia (CIN 1), to moderate dysplasia (CIN 2) and severe dysplasia/carcinoma in situ (CIN 3. CIN 1 is also referred to as low grade squamous intraepithelial lesion (LSIL) and CIN 2 and CIN 3 together as high grade squamous intraepithelial lesion (HSIL). Cervical adenocarcinoma are preceded by the precancerous precursor lesions referred to as adenocarcinoma in situ (ACIS). Precancerous cervical lesions are in principle detectable by e.g. cytology and treatable and cervical cancer is therefore considered a preventable disease. Cervical screening is aimed to early diagnose the high-grade precancerous lesions (CIN2, CIN3, ACIS) in order to prevent the development of cervical cancer and of treatable cancerous lesions. In preferred embodiments, precancerous lesions of cervical cancer CIN1, CIN2, CIN3 and ACIS are detected with a method of the invention, more preferably CIN2 and CIN3, most preferably CIN3. In further preferred embodiments, cervical squamous cell carcinomas (cSCC) and/or cervical adenocarcinoma (cAdC) or a risk of any thereof is detected with a method of the invention.

Vulvar cancer can both be HPV induced (about 30-40%) and non-HPV induced. High-grade vulvar intraepithelial neoplasia (VIN) is the considered the precancerous lesion of vulvar squamous cell carcinoma (vSCC), but the majority of VIN do not proceed to vSCC. VIN is divided into high-grade squamous intraepithelial lesion (HSIL), which is related to HPV, and differentiated VIN (dVIN), which is independent of HPV. In preferred embodiments, precancerous lesions of cancer vulvar intraepithelial neoplasia (VIN) and/or vulvar high grade squamous intraepithelial lesion (vHSIL) are detected with a method of the invention. In further preferred embodiments, vulvar carcinoma, more preferably vulvar squamous cell carcinomas (vSCC), or a risk of any thereof is detected with a method of the invention.

Endometrial cancer is the most common gynaecologic malignancy in many developed countries. Early stage endometrial cancer has a very good prognosis, but early detection strategies for endometrial cancer are lacking. Most endometrial cancers are low-grade and are preceded by hyperplasia precursors. However, grade 3 endometrioid, serous, clear cell and carcinosarcoma endometrial cancer are associated with high mortality. Endometrial cancers spontaneously shed tumor cells, including in cervical scrapes, and non-invasive sampling for early detection of endometrial cancer is therefore possible. In preferred embodiments, endometrioid, clear cell, carcinosarcoma and/or serous endometrial cancer or a risk thereof is detected with a method of the invention, more preferably endometrioid and/or serous endometrial cancer or a risk thereof.

Ovarian cancer is amongst the gynaecological cancers with the highest mortality. Early detection is the most important factor for long-term survival. The three main types of ovarian cancer are epithelial, germ cell and sex cord stromal ovarian cancer. Epithelial ovarian cancer is further divided into mucinous, clear cell, serous and endometrioid, transitional and undifferentiated ovarian cancer. In preferred embodiments, epithelial ovarian cancer or a risk thereof is detected with a method of the invention. In further preferred embodiments, mucinous, clear cell, serous and/or endometrioid ovarian cancer or a risk thereof is detected with a method of the invention.

As used herein an individual “at risk of suffering from cancer” or “predisposed to cancer” preferably means that the individual is an individual having a precancerous lesion or precancerous lesions, in particular high-grade precancerous lesions. Precancerous lesions are for instance cervical intraepithelial neoplasia (CIN)2 or CIN3, adenocarcinoma in situ (ACIS), vulvar intraepithelial neoplasia (VIN), and vulvar high grade squamous intraepithelial lesion (HSIL). Hence, in preferred embodiments, a method of the invention comprises determining whether said individual is suffering from a precancerous lesion, in particular CIN2, CIN3, ACIS, VIN, and/or HSIL.

As used herein, a methylated nucleic acid molecule or sequence is a nucleic acid molecule or sequence that contains one or more methylated nucleotides. A gene or promoter region or chromosomal region comprising at least one methylated nucleotide can be considered methylated. I.e. the methylation status of the gene or promoter region or chromosomal region is methylated. A gene or promoter region or chromosomal region that does not comprise any methylated nucleotides is unmethylated.

As used herein, a methylated nucleotide refers to the presence of a methyl moiety on a nucleotide, in particular on cytosine. Cytosine does not contain a methyl moiety on its pyrimidine ring, however 5-methylcytosine contains a methyl moiety at position 5 of its pyrimidine ring. The latter is herein also referred to as a methylated cytosine.

As used herein “promoter region” refers to a nucleotide sequence capable of controlling the expression of a genetic coding sequence. A promoter sequence is typically located 5′ of a coding sequence. As is known to a person skilled in the art, different promoters may direct the expression of a gene in different tissues or cell types, or at different stages of development, or in response to different environmental conditions.

“CpG sites” or “CG sites” are regions of DNA where a cytosine nucleotide is followed by a guanine nucleotide in the linear sequence of bases along its 5′→3′ direction. CpG sites occur with high frequency in genomic regions called CpG islands (or CG islands). In many genes, CpG islands begin just upstream of a promoter and extend into the coding sequence, although CpG islands can also be found in multiple regions of a gene or non-coding chromosomal region.

In a first aspect, the invention provides a method of identifying a gynaecological or ano-uro-genital tissue or cell sample or a tonsil, skin, blood, plasma, serum, saliva, serous fluid, urine or stool sample as indicative of cancer or as indicative of a predisposition to cancer, comprising determining the methylation status of one or more genes and/or promoter regions thereof and/or one or more chromosomal regions selected from table 1.

Also provided is a method comprising determining methylation status of one or more genes and/or promoter regions thereof and/or one or more chromosomal regions selected from table 1 in a test sample

The novel multi-cancer methylation markers of the present inventions are listed in table 1. The novel methylation markers were found to be multi-cancer predisposition markers, i.e. they are markers for multiple types of cancer. These markers are therefore herein also referred as multi-cancer markers.

As used herein the term “multi-cancer marker” indicates that the marker is a marker for multiple different types of cancer and/or their precancerous lesions, preferably at least three different types of cancer and/or their precancerous lesions, more preferably at least four different types of cancer and/or their precancerous lesions, more preferably at least five different types of cancer and/or their precancerous lesions, without distinguishing between said different cancers. Multi-cancer markers are also referred to as “pan-cancer”, “pan” and “general” markers. Said different types of cancer and their precancerous lesions are preferably, or are preferably selected from, gynaecological, ano-uro-genital, head and neck (in particular tonsil) or cutaneous cancers and their precancerous lesions. In preferred embodiments, said different types of cancer and their precancerous lesions are, or are selected from, cervical, vulvar, endometrial, ovarian, fallopian tube, vaginal, colon, colorectal, anal, head and neck (in particular tonsil), penile, bladder, prostate or cutaneous cancer and precancerous lesions thereof. In further preferred embodiments, said different types of cancer and their precancerous lesions are, or are selected from, cervical, vulvar, endometrial, ovarian, fallopian tube, head/neck (in particular tonsil), cutaneous, colorectal and anal cancer and precancerous lesions thereof. In further preferred embodiments, said different types of cancer and their precancerous lesions are, or are selected from, cervical, vulvar, endometrial, ovarian, fallopian tube, head/neck (in particular tonsil) or colorectal cancer and precancerous lesions thereof. In some preferred embodiments, said different types of cancer and their precancerous lesions are, or are selected from, HPV induced cervical, vulvar, vaginal, anal, head and neck (in particular tonsil), penile, or cutaneous cancer and precancerous lesions. In other preferred embodiments, said different types of cancer and their precancerous lesions are, or are selected from, non-HPV induced cervical, vulvar, endometrial, ovarian, fallopian tube, vaginal, colon, colorectal, anal, penile, bladder and prostate cancer or precancerous lesions.

Because they are multi-cancer markers, the markers of the present invention are excellent for first screening of test samples from individuals. For instance, the makers are suitable as triage test for in individuals with a positive HPV screening result or are otherwise designated for cancer testing, and are able to distinguish individuals who are suffering from or predisposed to cancer from individuals who are not suffering from or predisposed to cancer. Individuals identified as suffering from or predisposed to cancer are for instance referred to medical practitioners for further testing to identify the type of cancer, if appropriate, and/or for treatment. Hence, in preferred embodiments of a method of identifying a gynaecological or ano-uro-genital tissue or cell sample or a tonsil, skin, blood, plasma, serum, saliva, serous fluid, urine or stool sample as indicative of cancer or as indicative of a predisposition to cancer, the type of said cancer is unidentified or not identified, in particular not identified using the methylation markers listed in Table 1. In preferred embodiments the cancer is cervical, vulvar, endometrial, ovarian, fallopian tube, vaginal, colon, colorectal, anal, head and neck (in particular tonsil), penile, bladder, prostate and cutaneous cancer, preferably cervical, vulvar, endometrial, ovarian, fallopian tube, head/neck (in particular tonsil), cutaneous, colorectal or anal cancer, but the type of said cancer is unidentified or not identified, in particular not identified using the methylation markers listed in Table 1. Although the markers are designed as multi-markers or a multi-marker panel, the markers individually and in combinations have a sensitivity and specificity that is equal to or higher than that of cancer-specific marker.

TABLE 1
List of general methylation markers (multi-
cancer markers) of the invention.

| | chromo- | gene / | Chromosomal | |
|---|---|---|---|---|
| no | some | region | location (hg38) | Ensembl ID |
| 1 | chr3 | METloc002 | chr3: 147384749- | |
| | | | 147385097 | |
| 2 | chr1 | METloc001 | chr1: 145475713- | |
| | | | 145476399 | |
| 3 | chr9 | ARID3C | chr9: 34623552- | ENSG00000205143 |
| | | | 34624056 | |
| 4 | chr17 | ARL5C | chr17: 39165088- | ENSG00000141748 |
| | | | 39165444 | |
| 5 | chr1 | METloc003 | chr1: 148048856- | |
| | | | 148049542 | |
| 6 | chr13 | METloc004 | chr13: 112056904- | |
| | | | 112057851 | |
| 7 | chr7 | METloc005 | chr7: 101302849- | |
| | | | 101303191 | |
| 8 | chr7 | METloc006 | chr7: 35261398- | |
| | | | 35262258 | |
| 9 | chr22 | TPTEP1 | chr22: 16601841- | ENSG00000100181 |
| | | | 16602129 | |
| 10 | chr1 | METloc007 | chr1: 50415661- | |
| | | | 50416003 | |
| 11 | chr7 | METloc008 | chr7: 35257718- | |
| | | | 35258013 | |

Accordingly, in preferred embodiments of a method of the invention, the one or more genes and/or promoter regions thereof and/or one or more chromosomal regions selected from table 1 are hypermethylated in at least two of the following cancers and/or their precancerous lesions: cervical, vulvar, endometrial, ovarian, fallopian tube, vaginal, colon, colorectal, anal, head and neck, penile, bladder, prostate and cutaneous cancer, more preferably cervical, vulvar, endometrial, ovarian, fallopian tube, head/neck (in particular tonsil), cutaneous, colorectal and anal cancer and precancerous lesions thereof, more preferably cervical, vulvar, endometrial, ovarian, fallopian tube, head/neck (in particular tonsil) and colorectal cancer. In further preferred embodiments of a method of the invention, the one or more genes and/or promoter regions thereof and/or one or more chromosomal regions selected from table 1 are hypermethylated in at least three, more preferably at least four, more preferably at least five, more preferably all of said cancers and/or their precancerous lesions.

Accordingly, the invention therefore further provides a use of one or more genes and/or promoter regions thereof and/or one or more chromosomal regions selected from table 1 as a multi-cancer marker for a gynaecological, ano-uro-genital, head and neck or cutaneous cancer and precancerous lesions thereof. Also provided is a use of one or more genes and/or promoter regions thereof and/or one or more chromosomal regions selected from table 1 as a multi-cancer marker for detection of cancer or a precancerous lesion in a gynaecological or ano-uro-genital tissue or cell sample or a tonsil, skin, blood, plasma, serum, saliva, serous fluid, urine or stool sample. Also provided is a use of one or more genes and/or promoter regions thereof and/or one or more chromosomal regions selected from table 1 as a multi-cancer marker for identifying a gynaecological or ano-uro-genital tissue or cell sample or a tonsil, skin, blood, plasma, serum, saliva, serous fluid, urine or stool sample as indicative of cancer or as indicative of a predisposition to cancer. Also provided is a use of one or more primers and/or probes specific for the detection of DNA methylation in one or more genes and/or promoter regions thereof and/or one or more chromosomal regions selected from table 1 as a multi-cancer marker for gynaecological, ano-uro-genital, head and neck or cutaneous cancer and precancerous lesions thereof. In preferred embodiments the type of said cancer is unidentified or not identified, in particular not identified using the methylation markers listed in Table 1. The gynaecological, ano-uro-genital, head and neck or cutaneous cancer and precancerous lesions thereof are preferably or are preferably selected from, cervical, vulvar, endometrial, ovarian, fallopian tube, vaginal, colon, colorectal, anal, head and neck, penile, bladder, prostate and cutaneous cancer, more preferably from cervical, vulvar, endometrial, ovarian, fallopian tube, head/neck, cutaneous, colorectal and anal cancer and precancerous lesions thereof, more preferably from cervical, vulvar, endometrial, ovarian, fallopian tube, head/neck and colorectal cancer. However, in preferred embodiments the type of said cancer is unidentified or not identified, in particular not identified using the methylation markers listed in Table 1.

The methods, uses and methylation markers of the invention are particularly suitable for identifying or classifying individuals as suffering from or being at risk of suffering from cancer, in particular a gynaecological, ano-uro-genital, head and neck or cutaneous cancer and precancerous lesions thereof, more in particular cervical, vulvar, endometrial, ovarian, fallopian tube, vaginal, colon, colorectal, anal, head and neck, penile, bladder, prostate and cutaneous cancer, more preferably from cervical, vulvar, endometrial, ovarian, fallopian tube, head/neck, cutaneous, colorectal and anal cancer and precancerous lesions thereof, more preferably from cervical, vulvar, endometrial, ovarian, fallopian tube, head/neck and colorectal cancer. The invention therefore further provides a method for classifying an individual as suffering from or being at risk of suffering from cancer, in particular a gynaecological, ano-uro-genital, head and neck or cutaneous cancer, more in particular cervical, vulvar, endometrial, ovarian, fallopian tube, vaginal, colon, colorectal, anal, head and neck, penile, bladder, prostate and cutaneous cancer, more preferably from cervical, vulvar, endometrial, ovarian, fallopian tube, head/neck, cutaneous, colorectal and anal cancer and precancerous lesions thereof, more preferably from cervical, vulvar, endometrial, ovarian, fallopian tube, head/neck and colorectal cancer, the method comprising determining DNA methylation of one or more genes and/or promoter regions thereof and/or one or more chromosomal regions selected from table 1 in a sample of said individual, and classifying said individual based on said DNA methylation.

In some embodiments, a method of the invention comprises classifying an individual as being at risk of suffering from cancer. This preferably comprises determining whether the individual is an individual having a precancerous lesion or precancerous lesions. In preferred embodiments, said precancerous lesions are cervical, vulvar, endometrial or ovarian precancerous lesions, more preferably cervical or vulvar precancerous lesions.

Further provided is a method for analysing a sample of an individual, the method comprising determining DNA methylation of one or more genes and/or promoter regions thereof and/or one or more chromosomal regions selected from table 1 in a said sample. In preferred embodiments said method comprises typing said sample based on said DNA methylation.

The invention further provides a method for determining methylation status of one or more genes and/or promoter regions thereof and/or one or more chromosomal regions selected from table 1 in a sample of an individual. Said sample is preferably a gynaecological or ano-uro-genital tissue or cell sample or a tonsil, skin, blood, plasma, serum, saliva, serous fluid, urine or stool sample. In preferred embodiments, said sample as defined herein is a self-sample.

In addition to the multi-cancer markers listed in table 1, the present inventors have identified novel cancer-specific methylation markers. These are listed in Table 1A. It was found that chromosomal region METloc009 is a specific methylation marker for cervical and vulvar cancer and colorectal and anal cancer, GLIS3 is a specific methylation marker for vulvar cancer and FRMD4B is a specific marker for cervical adenocarcinoma. As demonstrated in the Examples herein, all three specific methylation markers listed in Table 1A have an excellent specificity and sensitivity and other characteristics (see Table 3). In particular, FRMD4B has both a high sensitivity (0.92) and specificity (0.97) for detecting cervical adenocarcinoma.

The invention therefore further provides a method for classifying an individual as suffering from or being at risk of suffering from cervical cancer or vulvar cancer or precancerous lesion thereof or colorectal or anal cancer or precancerous lesion thereof, the method comprising determining DNA methylation of chromosomal region METloc009 as indicated in table 1A, GLIS3 gene or a promoter region thereof and/or FRMD4B gene or a promoter region thereof in a sample of said individual, and classifying said individual based on said DNA methylation.

Also provided is a method for determining methylation status of chromosomal region METloc009 as indicated in table 1A, GLIS3 gene or a promoter region thereof and/or FRMD4B gene or a promoter region thereof in a sample of an individual, the method preferably comprising contacting said sample with means for detecting methylation status of said METloc009, GLIS3 gene or a promoter region thereof and/or FRMD4B gene or a promoter region thereof.

Also provided is a method for detecting cervical cancer or vulvar cancer or precancerous lesion thereof or colorectal or anal cancer or precancerous lesion thereof, the method comprising detecting hypermethylation in chromosomal region METloc009 as indicated in table 1A, GLIS3 gene or a promoter region thereof and/or FRMD4B gene or a promoter region thereof.

In preferred embodiments of these methods detection of hypermethylation in chromosomal region METloc009 as indicated in table 1A and/or GLIS3 gene or a promoter region thereof indicates the presence of vulvar cancer or a precancerous lesion, in particular vulvar cancer.

In further preferred embodiments of these methods, detection of hypermethylation in chromosomal region METloc009 as indicated in table 1A indicates the presence of cervical cancer or a precancerous lesion, in particular cervical cancer.

In further preferred embodiments of these methods, detection of hypermethylation in chromosomal region METloc009 as indicated in table 1A indicates the presence of colorectal or anal cancer or precancerous lesion thereof, in particular colorectal or anal cancer.

In further preferred embodiments of these methods, detection of hypermethylation in FRMD4B gene or a promoter region thereof indicates the presence of cervical adenocarcinoma.

Also provided is a use of one or more primers and/or probes specific for the detection of DNA methylation in chromosomal region METloc009 as indicated in table 1A, GLIS3 gene or a promoter region thereof and/or FRMD4B gene or a promoter region thereof as a marker for detecting cervical cancer or vulvar cancer or precancerous lesion thereof or colorectal or anal cancer or precancerous lesion thereof.

TABLE 1A
List of specific methylation markers of the invention.

| | chromo- | Gene / | Chromosomal | |
|---|---|---|---|---|
| No. | some | region | locatie (hg38) | Ensemble ID |
| 1 | chr10 | METloc009 | chr10: 23173346- | |
| | | | 23173626 | |
| 2 | chr9 | GLIS3 | chr9: 4300003- | ENSG00000107249 |
| | | | 4300291 | |
| 3 | chr3 | FRMD4B | chr3: 69542419- | ENSG00000114541 |
| | | | 69542699 | |

In some embodiments, the methods and uses of the invention comprise determining the DNA methylation and/or methylation status of one or more genes and/or promoter regions thereof and/or one or more chromosomal regions selected from table 1 or table 1A. The genes and/or promoter regions thereof and the chromosomal regions listed in table 1 have been identified by the present inventors as markers whose methylation status is linked to cancer, in particular a gynaecological, ano-uro-genital, head and neck or cutaneous cancer, more in particular cervical, vulvar, endometrial, ovarian, fallopian tube, vaginal, colon, colorectal, anal, head and neck, penile, bladder, prostate and cutaneous cancer, more in particular cervical, vulvar, endometrial, ovarian, fallopian tube, head/neck, cutaneous, colorectal and anal cancer and precancerous lesions thereof, more in particular cervical, vulvar, endometrial, ovarian, fallopian tube, head/neck and colorectal cancer. The genes and/or promoter regions thereof and the chromosomal region listed in table 1A have been identified by the present inventors as specific methylation markers for cervical and vulvar cancer and colorectal and anal cancer (METloc009), vulvar cancer (GLIS3) or cervical adenocarcinoma (FRMD4B). These genes or their promoter regions and chromosomal regions listed in table 1 and 1A are herein also referred as methylation markers. Analyses of the methylation status of these genes, promoter regions and chromosomal regions has not been reported before, nor have they been reported as a methylation marker for disease in general or cancer. The present inventors for the first time demonstrate that the methylation status of these genes, promoter regions and chromosomal regions has value as marker and prognostic marker In particular as multi-cancer markers for the genes, promoters and chromosomal regions of table 1, in particular for a gynaecological, ano-uro-genital, head and neck or cutaneous cancer, more in particular cervical, vulvar, endometrial, ovarian, fallopian tube, vaginal, colon, colorectal, anal, head and neck, penile, bladder, prostate and cutaneous cancer, more in particular cervical, vulvar, endometrial, ovarian, fallopian tube, head/neck, cutaneous, colorectal and anal cancer and precancerous lesions thereof, more in particular cervical, vulvar, endometrial, ovarian, fallopian tube, head/neck and colorectal cancer, or as specific methylation marker for the genes, promoters and chromosomal regions of table 1A.

Suitable methods for determining methylation status of genes, promoter regions and chromosomal regions are known in the art and detailed herein below. A method for determining methylation status of said one or more genes, promoter regions and/or chromosomal regions preferably comprises contacting said sample with means for detecting methylation status of said one or more genes and/or promoter regions thereof and/or one or more chromosomal regions selected from table 1 or 1A. In preferred embodiments, said means for detecting methylation status of genes, promoter regions thereof or chromosomal regions as defined herein comprise primers and/or probes specific for said gene, promoter region or chromosomal region. Said contacting step preferably comprises adding primers and/or probes to a sample comprising nucleic acid, preferably DNA.

Also provided is therefore a method of determining methylation status of one or more genes and/or promoter regions thereof and/or one or more chromosomal regions selected from table 1 or table 1A in a test sample comprising using methylation specific PCR primers and/or probes in methylation specific PCR to determine methylation status of the one or more genes and/or promoter regions thereof and/or one or more chromosomal regions selected from table 1 or table 1A.

The genes indicated in table 1 and 1A are identified by their Ensembl id or chromosomal location if no Gene name/id or Ensembl id is available. Chromosomal location can be found at the National Center for Biotechnology Information (http://www.ncbi.nlm.nih.gov) and are readily identifiable by a skilled person.

A skilled person will understand that functionally relevant variants of each of the gene, promoter or chromosomal region sequences may also be detected according to the methods and uses of the invention. For example, the methylation status of splice variants of the listed genes or of sequences containing single nucleotide polymorphisms (SNP) may be determined or detected according to the methods of the invention. Variant sequences preferably have at least 90%, at least 91%, at least 92%, at least 93%, at least 94%, at least 95%, at least 96%, at least 97%, at least 98%, or at least 99% nucleotide sequence identity with the nucleotide sequences indicated in the tables herein. Programs for determining percentage nucleotide sequence identity are available in the art, including the Basic Local Alignment Search Tool (BLAST) available from the National Center for Biotechnology Information.

The chromosomal regions listed in table 1 and table 1A are identified based on Homo sapiens (human) genome assembly GRCh38 (hg38). The tables indicate the location of the chromosomal region by chromosome id, start position, end position and location which is a concatenation of the three previously mentioned indicators.

The chromosomal regions listed in table 1 and table 1A are referred to as “METloc”, which is an abbreviation of “methylation location”, followed by a numerical value. Any reference to these regions as METloc001, METloc002, etc. is a reference to the relevant chromosomal location indicated in table 1 or table 1A.

As used herein methylation or hypermethylation is DNA methylation or DNA hypermethylation. As demonstrated in the Examples herein, it was found that differential methylation of the methylation markers listed in table 1 is able to differentiate multiple types of gynaecological, ano-uro-genital, head and neck or cutaneous cancers and precancerous lesions. Additionally, differential methylation of the methylation markers listed in table 1A is able to differentiate specific types of cancer, as described herein. In preferred embodiments, determining methylation status comprises determining differential methylation. In preferred embodiments, differential methylation of the one or more genes, promoter regions and/or chromosomal regions is indicative of cancer as defined herein or a predisposition to cancer as defined herein. In further preferred embodiments, hypermethylation of the one or more genes, promoter regions or chromosomal regions is indicative of cancer as defined herein or a predisposition to cancer as defined herein.

In preferred embodiments, methylation status and/or differential methylation of the methylation markers of the invention is determined and/or detected in the chromosomal location or one of the chromosomal locations as indicated in table 1 or table 1A for the relevant marker or markers. E.g. the methylation status and/or differential methylation of the marker METloc001 is preferably determined or detected within the location chr1:145475713-145476399. As another example, the methylation status and/or differential methylation of the marker ARID3C is preferably determined or detected within the location chr9:34623552-34624056.

In particular, it was found by the present inventors that hypermethylation of the genes, their promoter regions and chromosomal regions in table 1 and table 1A is indicative for cancer and prognostic for cancer. “hypermethylation,” when used in reference to a gene, a gene region, or a CpG island as described herein. As used herein “hypermethylation” means that methylation of nucleotides is found in relative abundance. It preferably indicates that the methylation status of the marker in a cell, sample or test sample of an individual that is suffering from or at risk of suffering from a cancer as defined herein is higher than the methylation status of the marker in a reference sample as defined herein below, in particular in a similar cell or sample of an individual that is not suffering from or at risk of suffering from said cancer. The term hypermethylation includes any methylation of cytosine at a position that is normally unmethylated in the relevant gene, promoter region thereof or chromosomal region, i.e. unmethylated in a reference cell or sample as defined herein below, in particular in a cell or sample of an individual that is not suffering from or at risk of suffering from said cancer.

Accordingly, a method of the invention preferably comprises determining whether said marker genes, their promoter regions and/or chromosomal regions are hypermethylated, whereby the presence of such hypermethylation of the genes, promoter region and/or chromosomal regions listed in table 1 indicates the presence or risk of a gynaecological, ano-uro-genital, head and neck or cutaneous cancer, in particular a cervical, vulvar, endometrial, ovarian, fallopian tube, vaginal, colon, colorectal, anal, head and neck, penile, bladder, prostate or cutaneous cancer, more in particular cervical, vulvar, endometrial, ovarian, fallopian tube, head/neck, cutaneous, colorectal and anal cancer and precancerous lesions thereof, more in particular cervical, vulvar, endometrial, ovarian, fallopian tube, head/neck and colorectal cancer. The presence of such hypermethylation of the genes, promoter region and/or chromosomal region listed in table 1A indicates the presence or risk of cervical and vulvar cancer or colorectal and anal cancer (for METloc009), vulvar cancer (for GLIS3 or promoter region) or cervical adenocarcinoma (FRMD4B or promoter region).

In preferred embodiments, hypermethylation of the methylation markers of the invention is determined and/or detected in the chromosomal location or one of the chromosomal locations as indicated in table 1 or table 1A for the relevant marker.

In preferred embodiments, the methylation status or DNA methylation, in particular hypermethylation, of at least chromosomal region METloc001 is determined or detected, preferably within a location as defined in table 1 for METloc001, optionally in combination with methylation status or DNA methylation, in particular hypermethylation, of one or more other genes, their promoter regions and/or chromosomal regions listed in table 1. Analyses of the methylation status of this chromosomal region has not been reported before, nor has this chromosomal region been reported as a marker for disease in general or cancer, let alone that its methylation status is a marker for cancer or a predisposition to cancer. As demonstrated in the Example herein, it was found that hypermethylation of METloc001 is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Tables 2 and 4), and a particularly high sensitivity, specificity, positive predictive value, negative predictive value, accuracy and/or Matthews correlation coefficient as methylation marker for head/neck (tonsil) cancer (see Table 6), colorectal and anal cancer (see Table 7) and cutaneous cancer (see Table 8).

In other preferred embodiments, the methylation status or DNA methylation, in particular hypermethylation, of at least chromosomal region METloc002 is determined or detected, preferably within a location as defined in table 1 for METloc002, optionally in combination with methylation status or DNA methylation, in particular hypermethylation, of one or more other genes, their promoter regions and/or chromosomal regions listed in table 1. Analyses of the methylation status of this chromosomal region has not been reported before, nor has this chromosomal region been reported as a marker for disease in general or cancer, let alone that its methylation status is a marker for cancer or a predisposition to cancer. As demonstrated in the Example herein, it was found that hypermethylation of METloc002 is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Tables 2 and 4), and a particularly high sensitivity, specificity, positive predictive value, negative predictive value, accuracy and/or Matthews correlation coefficient as methylation marker for head/neck (tonsil) cancer (see Table 6), colorectal and anal cancer (see Table 7) and cutaneous cancer (see Table 8).

In other preferred embodiments, the methylation status or DNA methylation, in particular hypermethylation, of at least chromosomal region ARID3C (AT-rich interaction domain 3C) or a promoter region thereof is determined or detected, preferably within a location as defined in table 1 for ARID3C or a promoter region thereof, optionally in combination with methylation status or DNA methylation, in particular hypermethylation, of one or more other genes, their promoter regions and/or chromosomal regions listed in table 1. ARID3C is a member of the ARID (AT-rich interaction domain) family of proteins. Analyses of the methylation status of ARID3C and its promoter region thereof has not been reported before, nor has ARID3C and its promoter region been reported as a marker for disease in general or cancer, let alone that its methylation status is a multi-cancer marker for cancer or a predisposition to cancer. As demonstrated in the Example herein, it was found that hypermethylation of ARID3C or its promoter region is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Tables 2 and 4), and a particularly high sensitivity, specificity, positive predictive value, negative predictive value, accuracy and Matthews correlation coefficient as methylation marker for head/neck (tonsil) cancer (see Table 6) and colorectal and anal cancer (see Table 7).

In other preferred embodiments, the methylation status or DNA methylation, in particular hypermethylation, of at least chromosomal region ARL5C (ADP Ribosylation Factor Like GTPase 5C) or a promoter region thereof is determined or detected, preferably within a location as defined in table 1 for ARL5C or a promoter region thereof, optionally in combination with methylation status or DNA methylation, in particular hypermethylation, of one or more other genes, their promoter regions and/or chromosomal regions listed in table 1. As demonstrated in the Example herein, it was found that hypermethylation of ARL5C or its promoter region is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Tables 2 and 4), and a particularly high sensitivity, specificity, positive predictive value, negative predictive value, accuracy and Matthews correlation coefficient as methylation marker for head/neck (tonsil) cancer (see Table 6) and colorectal and anal cancer (see Table 7).

In other preferred embodiments, the methylation status or DNA methylation, in particular hypermethylation, of at least chromosomal region METloc003 is determined or detected, preferably within a location as defined in table 1 for METloc003, optionally in combination with methylation status or DNA methylation, in particular hypermethylation, of one or more other genes, their promoter regions and/or chromosomal regions listed in table 1. Analyses of the methylation status of this chromosomal region has not been reported before, nor has this chromosomal region been reported as a marker for disease in general or cancer, let alone that its methylation status is a marker for cancer or a predisposition to cancer. As demonstrated in the Example herein, it was found that hypermethylation of this chromosomal region is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Table 2).

In other preferred embodiments, the methylation status or DNA methylation, in particular hypermethylation, of at least chromosomal region METloc004 is determined or detected, preferably within a location as defined in table 1 for METloc004, optionally in combination with methylation status or DNA methylation, in particular hypermethylation, of one or more other genes, their promoter regions and/or chromosomal regions listed in table 1. Analyses of the methylation status of this chromosomal region has not been reported before, nor has this chromosomal region been reported as a marker for disease in general or cancer, let alone that its methylation status is a marker for cancer or a predisposition to cancer. As demonstrated in the Example herein, it was found that hypermethylation of this chromosomal region is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Table 2).

In other preferred embodiments, the methylation status or DNA methylation, in particular hypermethylation, of at least chromosomal region METloc005 is determined or detected, preferably within a location as defined in table 1 for METloc005, optionally in combination with methylation status or DNA methylation, in particular hypermethylation, of one or more other genes, their promoter regions and/or chromosomal regions listed in table 1. Analyses of the methylation status of this chromosomal region has not been reported before, nor has this chromosomal region been reported as a marker for disease in general or cancer, let alone that its methylation status is a marker for cancer or a predisposition to cancer. As demonstrated in the Example herein, it was found that hypermethylation of this chromosomal region is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Table 2).

In other preferred embodiments, the methylation status or DNA methylation, in particular hypermethylation, of at least chromosomal region METloc006 is determined or detected, preferably within a location as defined in table 1 for METloc006, optionally in combination with methylation status or DNA methylation, in particular hypermethylation, of one or more other genes, their promoter regions and/or chromosomal regions listed in table 1. Analyses of the methylation status of this chromosomal region has not been reported before, nor has this chromosomal region been reported as a marker for disease in general or cancer, let alone that its methylation status is a marker for cancer or a predisposition to cancer. As demonstrated in the Example herein, it was found that hypermethylation of this chromosomal region is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Table 2).

In other preferred embodiments, the methylation status or DNA methylation, in particular hypermethylation, of at least TPTEP1 gene (Transmembrane Phosphatase With Tensin Homology Pseudogene 1) or a promoter region thereof is determined or detected, preferably within a location as defined in table 1 for TPTEP1 or a promoter region thereof, optionally in combination with methylation status or DNA methylation, in particular hypermethylation, of one or more other genes, their promoter regions and/or chromosomal regions listed in table 1. Analyses of the methylation status of TPTEP1 and its promoter region thereof has not been reported before, nor has TPTEP1 and its promoter region been reported as a marker for disease in general or cancer, let alone that its methylation status is a multi-cancer marker for cancer or a predisposition to cancer. As demonstrated in the Example herein, it was found that hypermethylation of TPTEP1 or its promoter region is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Table 2).

In other preferred embodiments, the methylation status or DNA methylation, in particular hypermethylation, of at least chromosomal region METloc007 is determined or detected, preferably within a location as defined in table 1 for METloc007, optionally in combination with methylation status or DNA methylation, in particular hypermethylation, of one or more other genes, their promoter regions and/or chromosomal regions listed in table 1. Analyses of the methylation status of this chromosomal region has not been reported before, nor has this chromosomal region been reported as a marker for disease in general or cancer, let alone that its methylation status is a marker for cancer or a predisposition to cancer. As demonstrated in the Example herein, it was found that hypermethylation of this chromosomal region is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Table 2).

In other preferred embodiments, the methylation status or DNA methylation, in particular hypermethylation, of at least chromosomal region METloc008 is determined or detected, preferably within a location as defined in table 1 for METloc008, optionally in combination with methylation status or DNA methylation, in particular hypermethylation, of one or more other genes, their promoter regions and/or chromosomal regions listed in table 1. Analyses of the methylation status of this chromosomal region has not been reported before, nor has this chromosomal region been reported as a marker for disease in general or cancer, let alone that its methylation status is a marker for cancer or a predisposition to cancer. As demonstrated in the Example herein, it was found that hypermethylation of this chromosomal region is an excellent multi-cancer marker for gynaecological, ano-uro-genital, head and neck and cutaneous cancers that provides a particularly high sensitivity, specificity, positive predictive value, negative predictive value, accuracy and Matthews correlation coefficient as a multi-cancer methylation marker for determining the presence of cancer and predisposition to cancer (see Table 2).

In other preferred embodiments, the methylation status or DNA methylation, in particular hypermethylation, of at least chromosomal region METloc009 is determined or detected, preferably within a location as defined in table 1A for METloc009, optionally in combination with methylation status or DNA methylation, in particular hypermethylation, of GLIS3 gene or a promoter region thereof, preferably within a location as defined in table 1A for GLIS3. Analyses of the methylation status of this chromosomal region has not been reported before, nor has this chromosomal region been reported as a marker for disease in general or cancer, let alone that its methylation status is a marker for cervical and vulvar cancer and colorectal and anal cancer. As demonstrated in the Example herein, it was found that hypermethylation of this chromosomal region is an excellent marker for cervical and vulvar cancer, with a high sensitivity, specificity, positive predictive value, negative predictive value, accuracy and Matthews correlation coefficient (see Tables 3 and 9). It was further found that hypermethylation of this chromosomal region is an excellent marker for colorectal and anal cancer, with a high sensitivity, specificity, positive predictive value, negative predictive value, accuracy and Matthews correlation coefficient (see Table 10).

In other preferred embodiments, the methylation status or DNA methylation, in particular hypermethylation, of at least GLIS3 gene (GLIS Family Zinc Finger 3) or a promoter region thereof is determined or detected, preferably within a location as defined in table 1A for GLIS3, optionally in combination with methylation status or DNA methylation, in particular hypermethylation, of METloc009, preferably within a location as defined in table 1A for METloc009. Analyses of the methylation status of this gene or promoter region thereof has not been reported before, nor has this gene or promoter region thereof been reported as a marker for disease in general or cancer, let alone that its methylation status is a marker for cervical and vulvar cancer. As demonstrated in the Example herein, it was found that hypermethylation of this gene or promoter region thereof is an excellent marker for cervical and vulvar cancer, with a high sensitivity, specificity, negative predictive value, accuracy and Matthews correlation coefficient (see Tables 3 and 9).

In other preferred embodiments, the methylation status or DNA methylation, in particular hypermethylation, of at least FRMD4B gene (FERM Domain Containing 4B) or a promoter region thereof is determined or detected, preferably within a location as defined in table 1A for FRMD4B. Analyses of the methylation status of this gene or promoter region thereof has not been reported before, nor has this gene or promoter region thereof been reported as a marker for disease in general or cancer, let alone that its methylation status is a marker for cervical and vulvar cancer. As demonstrated in the Example herein, it was found that hypermethylation of this gene or promoter region thereof is an excellent marker for cervical and vulvar cancer, with a high sensitivity, specificity, negative predictive value, accuracy and Matthews correlation coefficient (see Tables 3 and 9).

In some preferred embodiments, the methylation status and/or DNA methylation of two or more of the genes, promoter regions thereof and chromosomal regions listed in table 1 is determined or detected, whereby the presence of such hypermethylation of one or both of said genes, promoter regions thereof and/or chromosomal regions indicates the presence or risk of a gynaecological, ano-uro-genital, head and neck or cutaneous cancer, in particular a cervical, vulvar, endometrial, ovarian, fallopian tube, vaginal, colon, colorectal, anal, head and neck, penile, bladder, prostate or cutaneous cancer, more in particular cervical, vulvar, endometrial, ovarian, fallopian tube, head/neck, cutaneous, colorectal and anal cancer and precancerous lesions thereof, more in particular cervical, vulvar, endometrial, ovarian, fallopian tube, head/neck and colorectal cancer. In some preferred embodiments, the methylation status of three or more, four or more, five or more of the genes, promoter regions thereof and chromosomal regions listed in table 1 is determined or detected, whereby the presence of such hypermethylation of one or more of said genes, promoter regions thereof and/or chromosomal regions indicates the presence or risk of a gynaecological, ano-uro-genital, head and neck or cutaneous cancer, in particular a cervical, vulvar, endometrial, ovarian, fallopian tube, vaginal, colon, colorectal, anal, head and neck, penile, bladder, prostate or cutaneous cancer, more in particular cervical, vulvar, endometrial, ovarian, fallopian tube, head/neck, cutaneous, colorectal and anal cancer and precancerous lesions thereof, more in particular cervical, vulvar, endometrial, ovarian, fallopian tube, head/neck and colorectal cancer.

In some preferred embodiments, methylation status or DNA methylation, in particular hypermethylation, of chromosomal locations METloc001 or METloc002 or ARID3C or ARL5C or a promoter region of ARID3C or ARL5C is determined, in combination with methylation status of one or more other genes, their promoter regions and/or chromosomal regions listed in table 1, more preferably of METloc001 or METloc002 or ARID3C or a promoter region thereof in combination with methylation status of one or more other genes, their promoter regions and/or chromosomal regions listed in table 1.

In further preferred embodiments, the methylation status and/or DNA methylation, in particular hypermethylation, of chromosomal location METloc001 and METloc002, preferably within a region as defined in table 1 for METloc001 and METloc002, and optionally of one or more other genes, promoter regions thereof and chromosomal regions listed in table 1 is determined or detected. As demonstrated in Table 5 the combination of METloc001 and METloc002 provides a particularly high sensitivity, specificity, positive predictive value, negative predictive value, accuracy and Matthews correlation coefficient as a multi-cancer methylation marker for determining the presence of cancer and predisposition to cancer, in particular for detecting vulvar, cervical, endometrial, fallopian tube and ovarian cancer.

In further preferred embodiments, the methylation status and/or DNA methylation, in particular hypermethylation, of METloc001 and ARID3C or a promoter region thereof, preferably within a region as defined in table 1 for METloc001 and ARID3C or a promoter region thereof, and optionally of one or more other genes, promoter regions thereof and chromosomal regions listed in table 1 is determined or detected. As demonstrated in Table 5 the combination of METloc001 and ARID3C provides a particularly high sensitivity, specificity, positive predictive value, negative predictive value, accuracy and Matthews correlation coefficient as a multi-cancer methylation marker for determining the presence of cancer and predisposition to cancer, in particular for detecting vulvar, cervical, endometrial, fallopian tube and ovarian cancer.

In further preferred embodiments, the methylation status and/or DNA methylation, in particular hypermethylation, of METloc001 and ARL5C or a promoter region thereof, preferably within a region as defined in table 1 for METloc001 and ARL5C or a promoter region thereof, and optionally of one or more other genes, promoter regions thereof and chromosomal regions listed in table 1 is determined or detected. As demonstrated in Table 5 the combination of METloc001 and ARL5C provides a particularly high sensitivity, specificity, positive predictive value, negative predictive value, accuracy and Matthews correlation coefficient as a multi-cancer methylation marker for determining the presence of cancer and predisposition to cancer, in particular for detecting vulvar, cervical, endometrial, fallopian tube and ovarian cancer.

In further preferred embodiments, the methylation status and/or DNA methylation, in particular hypermethylation, of METloc001 and METloc003, preferably within a region as defined in table 1 for METloc001 and METloc003, and optionally of one or more other genes, promoter regions thereof and chromosomal regions listed in table 1 is determined or detected.

In further preferred embodiments, the methylation status and/or DNA methylation, in particular hypermethylation, of METloc001 and METloc004, preferably within a region as defined in table 1 for METloc001 and METloc004, and optionally of one or more other genes, promoter regions thereof and chromosomal regions listed in table 1 is determined or detected.

In further preferred embodiments, the methylation status and/or DNA methylation, in particular hypermethylation, of METloc001 and METloc005, preferably within a region as defined in table 1 for METloc001 and METloc005, and optionally of one or more other genes, promoter regions thereof and chromosomal regions listed in table 1 is determined or detected.

In further preferred embodiments, the methylation status and/or DNA methylation, in particular hypermethylation, of METloc001 and METloc006, preferably within a region as defined in table 1 for METloc001 and METloc006, and optionally of one or more other genes, promoter regions thereof and chromosomal regions listed in table 1 is determined or detected.

In further preferred embodiments, the methylation status and/or DNA methylation, in particular hypermethylation, of METloc001 and TPTEP1 or a promoter region thereof, preferably within a region as defined in table 1 for METloc001 and TPTEP1 or a promoter region thereof, and optionally of one or more other genes, promoter regions thereof and chromosomal regions listed in table 1 is determined or detected.

In further preferred embodiments, the methylation status and/or DNA methylation, in particular hypermethylation, of METloc001 and METloc007, preferably within a region as defined in table 1 for METloc001 and METloc007, and optionally of one or more other genes, promoter regions thereof and chromosomal regions listed in table 1 is determined or detected.

In further preferred embodiments, the methylation status and/or DNA methylation, in particular hypermethylation, of METloc001 and METloc008, preferably within a region as defined in table 1 for METloc001 and METloc008, and optionally of one or more other genes, promoter regions thereof and chromosomal regions listed in table 1 is determined or detected.

In further preferred embodiments, the methylation status and/or DNA methylation, in particular hypermethylation, of METloc002 and ARID3C or a promoter region thereof, preferably within a region as defined in table 1 for METloc002 and ARID3C or a promoter region thereof, and optionally of one or more other genes, promoter regions thereof and chromosomal regions listed in table 1 is determined or detected. As demonstrated in Table 5 the combination of METloc002 and ARID3C provides a particularly high sensitivity, specificity, positive predictive value, negative predictive value, accuracy and Matthews correlation coefficient as a multi-cancer methylation marker for determining the presence of cancer and predisposition to cancer, in particular for detecting vulvar, cervical, endometrial, fallopian tube and ovarian cancer.

In further preferred embodiments, the methylation status and/or DNA methylation, in particular hypermethylation, of METloc002 and ARL5C or a promoter region thereof, preferably within a region as defined in table 1 for METloc002 and ARL5C or a promoter region thereof, and optionally of one or more other genes, promoter regions thereof and chromosomal regions listed in table 1 is determined or detected. As demonstrated in Table 5 the combination of METloc002 and ARL5C provides a particularly high sensitivity, specificity, positive predictive value, negative predictive value, accuracy and Matthews correlation coefficient as a multi-cancer methylation marker for determining the presence of cancer and predisposition to cancer, in particular for detecting vulvar, cervical, endometrial, fallopian tube and ovarian cancer.

In further preferred embodiments, the methylation status and/or DNA methylation, in particular hypermethylation, of METloc002 and METloc003, preferably within a region as defined in table 1 for METloc002 and METloc003, and optionally of one or more other genes, promoter regions thereof and chromosomal regions listed in table 1 is determined or detected.

In further preferred embodiments, the methylation status and/or DNA methylation, in particular hypermethylation, of METloc002 and METloc004, preferably within a region as defined in table 1 for METloc002 and METloc004, and optionally of one or more other genes, promoter regions thereof and chromosomal regions listed in table 1 is determined or detected.

In further preferred embodiments, the methylation status and/or DNA methylation, in particular hypermethylation, of METloc002 and METloc005, preferably within a region as defined in table 1 for METloc002 and METloc005, and optionally of one or more other genes, promoter regions thereof and chromosomal regions listed in table 1 is determined or detected.

In further preferred embodiments, the methylation status and/or DNA methylation, in particular hypermethylation, of METloc002 and METloc006, preferably within a region as defined in table 1 for METloc002 and METloc006, and optionally of one or more other genes, promoter regions thereof and chromosomal regions listed in table 1 is determined or detected.

In further preferred embodiments, the methylation status and/or DNA methylation, in particular hypermethylation, of METloc002 and TPTEP1 or a promoter region thereof, preferably within a region as defined in table 1 for METloc002 and TPTEP1 or a promoter region thereof, and optionally of one or more other genes, promoter regions thereof and chromosomal regions listed in table 1 is determined or detected.

In further preferred embodiments, the methylation status and/or DNA methylation, in particular hypermethylation, of METloc002 and METloc007, preferably within a region as defined in table 1 for METloc002 and METloc007, and optionally of one or more other genes, promoter regions thereof and chromosomal regions listed in table 1 is determined or detected.

In further preferred embodiments, the methylation status and/or DNA methylation, in particular hypermethylation, of METloc002 and METloc008, preferably within a region as defined in table 1 for METloc002 and METloc008, and optionally of one or more other genes, promoter regions thereof and chromosomal regions listed in table 1 is determined or detected.

In further preferred embodiments, the methylation status and/or DNA methylation, in particular hypermethylation, of ARID3C or a promoter region thereof and ARTL5C or a promoter region thereof, preferably within a region as defined in table 1 for ARID3C or a promoter region thereof and ARTL5C or a promoter region thereof, and optionally of one or more other genes, promoter regions thereof and chromosomal regions listed in table 1 is determined or detected. As demonstrated in Table 5 the combination of ARID3C and ARL5C provides a particularly high sensitivity, specificity, positive predictive value, negative predictive value, accuracy and Matthews correlation coefficient as a multi-cancer methylation marker for determining the presence of cancer and predisposition to cancer, in particular for detecting vulvar, cervical, endometrial, fallopian tube and ovarian cancer.

In further preferred embodiments, the methylation status and/or DNA methylation, in particular hypermethylation, of ARID3C or a promoter region thereof and METloc003, preferably within a region as defined in table 1 for ARID3C or a promoter region thereof and METloc003, and optionally of one or more other genes, promoter regions thereof and chromosomal regions listed in table 1 is determined or detected.

In further preferred embodiments, the methylation status and/or DNA methylation, in particular hypermethylation, of ARID3C or a promoter region thereof and METloc004, preferably within a region as defined in table 1 for ARID3C or a promoter region thereof and METloc004, and optionally of one or more other genes, promoter regions thereof and chromosomal regions listed in table 1 is determined or detected.

In further preferred embodiments, the methylation status and/or DNA methylation, in particular hypermethylation, of ARID3C or a promoter region thereof and METloc005, preferably within a region as defined in table 1 for ARID3C or a promoter region thereof and METloc005, and optionally of one or more other genes, promoter regions thereof and chromosomal regions listed in table 1 is determined or detected.

In further preferred embodiments, the methylation status and/or DNA methylation, in particular hypermethylation, of ARID3C or a promoter region thereof and METloc006, preferably within a region as defined in table 1 for ARID3C or a promoter region thereof and METloc006, and optionally of one or more other genes, promoter regions thereof and chromosomal regions listed in table 1 is determined or detected.

In further preferred embodiments, the methylation status and/or DNA methylation, in particular hypermethylation, of ARID3C or a promoter region thereof and TPTEP1 or a promoter region thereof, preferably within a region as defined in table 1 for ARID3C or a promoter region thereof and TPTEP1 or a promoter region thereof, and optionally of one or more other genes, promoter regions thereof and chromosomal regions listed in table 1 is determined or detected.

In further preferred embodiments, the methylation status and/or DNA methylation, in particular hypermethylation, of ARID3C or a promoter region thereof and METloc007, preferably within a region as defined in table 1 for ARID3C or a promoter region thereof and METloc007, and optionally of one or more other genes, promoter regions thereof and chromosomal regions listed in table 1 is determined or detected.

In further preferred embodiments, the methylation status and/or DNA methylation, in particular hypermethylation, of ARID3C or a promoter region thereof and METloc008, preferably within a region as defined in table 1 for ARID3C or a promoter region thereof and METloc008, and optionally of one or more other genes, promoter regions thereof and chromosomal regions listed in table 1 is determined or detected.

In further preferred embodiments, the methylation status and/or DNA methylation, in particular hypermethylation, of ARL5C or a promoter region thereof and METloc003, preferably within a region as defined in table 1 for ARL5C or a promoter region thereof and METloc003, and optionally of one or more other genes, promoter regions thereof and chromosomal regions listed in table 1 is determined or detected.

In further preferred embodiments, the methylation status and/or DNA methylation, in particular hypermethylation, of ARL5C or a promoter region thereof and METloc004, preferably within a region as defined in table 1 for ARL5C or a promoter region thereof and METloc004, and optionally of one or more other genes, promoter regions thereof and chromosomal regions listed in table 1 is determined or detected.

In further preferred embodiments, the methylation status and/or DNA methylation, in particular hypermethylation, of ARL5C or a promoter region thereof and METloc005, preferably within a region as defined in table 1 for ARL5C or a promoter region thereof and METloc005, and optionally of one or more other genes, promoter regions thereof and chromosomal regions listed in table 1 is determined or detected.

In further preferred embodiments, the methylation status and/or DNA methylation, in particular hypermethylation, of ARL5C or a promoter region thereof and METloc006, preferably within a region as defined in table 1 for ARL5C or a promoter region thereof and METloc006, and optionally of one or more other genes, promoter regions thereof and chromosomal regions listed in table 1 is determined or detected.

In further preferred embodiments, the methylation status and/or DNA methylation, in particular hypermethylation, of ARL5C or a promoter region thereof and TPTEP1 or a promoter region thereof, preferably within a region as defined in table 1 for ARL5C or a promoter region thereof and TPTEP1 or a promoter region thereof, and optionally of one or more other genes, promoter regions thereof and chromosomal regions listed in table 1 is determined or detected.

In further preferred embodiments, the methylation status and/or DNA methylation, in particular hypermethylation, of ARL5C or a promoter region thereof and METloc007, preferably within a region as defined in table 1 for ARL5C or a promoter region thereof and METloc007, and optionally of one or more other genes, promoter regions thereof and chromosomal regions listed in table 1 is determined or detected.

In further preferred embodiments, the methylation status and/or DNA methylation, in particular hypermethylation, of ARL5C or a promoter region thereof and METloc008, preferably within a region as defined in table 1 for ARL5C or a promoter region thereof and METloc008, and optionally of one or more other genes, promoter regions thereof and chromosomal regions listed in table 1 is determined or detected.

In further preferred embodiments, the methylation status and/or DNA methylation, in particular hypermethylation, of METloc003 and METloc004, METloc003 and METloc005, METloc003 and METloc006, METloc003 and METloc007, METloc003 and METloc008, METloc003 and TPTEP1 or a promoter region thereof, METloc004 and METloc005, METloc004 and METloc006, METloc004 and METloc007, METloc004 and METloc008, METloc004 and TPTEP1 or a promoter region thereof, METloc005 and METloc006, METloc005 and METloc007, METloc005 and METloc008, METloc005 and TPTEP1 or a promoter region thereof, METloc006 and METloc007, METloc006 and METloc008, METloc006 and TPTEP1 or a promoter region thereof, METloc007 and METloc008, METloc007 and TPTEP1 or a promoter region thereof, or METloc008 and TPTEP1 or a promoter region thereof, preferably within a region as defined in table 1 for these genes, promoter region thereof and/or chromosomal regions, and optionally of one or more other genes, promoter regions thereof and chromosomal regions listed in table 1 is determined or detected.

In case the methylation status of a combination of more than one of the genes, their promoter region and/or chromosomal regions listed in table 1 is determined or detected, hypermethylation of at least one the markers is indicative for an individual suffering from gynaecological, ano-uro-genital, head and neck or cutaneous cancer as defined herein or an individual that is at risk of suffering from a gynaecological, ano-uro-genital, head and neck or cutaneous cancer as defined herein.

A sample or test sample may comprise a cancer cell, a pre-cancer cell, a proliferating cell, or any other cell wherein the presence of a gynaecological, ano-uro-genital, head and neck or cutaneous cancer as defined herein can be detected.

The sample or test sample to be analyzed in the methods of the invention can be a sample comprising cells or sample comprising cell-free nucleic acid, in particular cell-free DNA. In preferred embodiments, the sample is selected from the group consisting of a tissue sample, cell sample, blood sample, plasma sample, serum sample, saliva sample, urine sample or stool sample.

In further preferred embodiments, in particular if methylation of one or more multicancer markers listed in table 1 is detected or determined, the sample is selected from a cervical, vulvar, endometrial, ovarian, fallopian tube, head/neck, cutaneous, colorectal and anal sample. In further preferred embodiments, the sample is selected from a cervical, vulvar, endometrial, ovarian, fallopian tube, head/neck and colorectal sample, in particular from a cervical, vulvar, endometrial, ovarian, fallopian tube, head/neck and colorectal tissue or cells.

A tissue sample is for instance a cytology of biopsy sample, including liquid biopsy sample. Examples of tissue samples and cell samples are for instance tissue or cell scrapings or swabs, such as a cervical scraping or swab. In preferred embodiments, the tissue or cell sample is a gynaecological or ano-uro-genital tissue or cell sample. In preferred embodiments, the cell sample is a gynaecological or ano-uro-genital cell sample. Examples of tissues and cells are cervical tissue and cells, vulvar tissue or cells, endometrial tissue or cells, ovarian tissue or cells, fallopian tube tissue or cells, vaginal tissue or cells, colon tissue or cells, colorectal tissue or cells, anal tissue or cells, penile tissue or cells, bladder tissue or cells and prostate tissue or cells. In other embodiments, the tissue sample is a biopsy, such as a tonsil biopsy or a colorectal biopsy. In other embodiments, the sample is a liquid biopsy sample.

In preferred embodiments, the sample is selected from a cervical, vulvar, endometrial, ovarian, fallopian tube, head/neck, cutaneous, colorectal and anal tissue or cell sample. In further preferred embodiments, the sample is a cervical, vulvar, endometrial, ovarian, fallopian tube, head/neck or colorectal tissue or cell sample. In further preferred embodiments, the sample is a cervical tissue or cell sample.

In other preferred embodiments, the sample is a sample that can be obtained with non-invasive methods, such as cervical scrapes or swabs, urine, stool, saliva or blood.

In other preferred embodiments, the sample is a urine sample. A urine sample can be either a cell containing sample or a cell-free urine sample, e.g. a sample comprising cell-free DNA.

In preferred embodiments, the sample is a sample of a tissue that is relevant for the type of cancer that the individual is suspected to suffer from or suspected to be at risk of suffering from.

For instance, if an individual is suspected to be suffering from or at risk of suffering from cervical cancer, the sample is preferably a sample comprising cervical tissue or cells or cervical cell-free DNA, such as a cervical tissue sample, cervical or vaginal scraping, lavage or swab or a cell-containing or cell-free urine sample, more preferably a sample comprising cervical tissue or cells, such as a cytology sample, cervical or vaginal scraping, lavage or swab or a cell-containing urine sample, more preferably a cervical tissue sample or cervical or vaginal scraping, lavage or swab.

Similarly, if an individual is suspected to be suffering from or at risk of suffering from vulvar, endometrial, ovarian, fallopian tube, vaginal or bladder cancer, the sample is preferably a sample comprising vulvar, endometrial, ovarian, fallopian tube, vaginal or bladder tissue or cells or vulvar, endometrial, ovarian, fallopian tube, vaginal or bladder cell-free DNA, such as a cytology sample, a vaginal scraping, lavage or swab or a cell-containing or cell-free urine sample, more preferably a cytology sample, a vaginal scraping, lavage or swab or a cell-containing urine sample, more preferably a cytology sample or a vaginal scraping, lavage or swab.

Similarly, if an individual is suspected to be suffering from or at risk of suffering from colon, colorectal or anal cancer, the sample is preferably a sample comprising colon, colorectal or anal tissue or cells or colon, colorectal or anal cell-free DNA, such as a cytology sample or a cell-containing or cell-free stool sample, more preferably a colon, colorectal or anal tissue or cell sample.

Similarly, if an individual is suspected to be suffering from or at risk of suffering from penile or prostate cancer, the sample is preferably a sample comprising penile or prostate tissue or cells or penile or prostate cell-free DNA, such as a cytology sample or a cell-containing or cell-free urine sample, more preferably a penile or prostate tissue or cell sample or cell-containing urine sample, more preferably a penile or prostate tissue or cell sample.

Similarly, if an individual is suspected to be suffering from or at risk of suffering from head and neck cancer, the sample is preferably a sample comprising oral cavity or oropharynx tissue or cells or oral cavity or oropharynx cell-free DNA, such as a cytology sample or a scraping, lavage or swab from the oral cavity or oropharynx, or a cell-containing or cell-free saliva sample, more preferably an oral cavity or oropharynx sample or cell-containing saliva sample, more preferably a oral cavity or oropharynx tissue or cell sample.

Similarly, if an individual is suspected to be suffering from or at risk of suffering from fallopian tube cancer, the sample is preferably a sample comprising fallopian tube tissue or cells or fallopian tube cell-free DNA, such as a cytology sample or a cell-containing sample, more preferably a fallopian tube tissue or cell sample.

The sample can be a self-sample or a sample taken by a medical professional, e.g. a physician-taken sample. In preferred embodiments the sample is a self-sample. As used herein, the term “self-sample” refers to a self-collected sample, i.e. a sample that is collected by the individual that is screened for the present or absence or risk of cancer. Examples of self-samples are a self-collected cervical or vaginal scraping, swap or lavage, a urine sample or a stool sample and a scraping, lavage or swab from the oral cavity or oropharynx. A preferred self-sample is a cervical or vaginal scraping, swap or lavage.

Hypermethylation or methylation status of chromosomal region METloc009, preferably in the region as indicated in Table 1A, is preferably determined in a cervical sample, preferably tissue, cell or cell free DNA sample, if the individual is suspected of suffering from or at risk of suffering from cervical cancer, in a vulvar sample, preferably tissue, cell or cell free DNA sample, if the individual is suspected of suffering from or at risk of suffering from cervical cancer or in a colorectal or anal sample, preferably tissue, cell or cell free DNA sample, if the individual is suspected expected to be suffering from or at risk of suffering from colorectal or anal cancer.

Hypermethylation or methylation status of GLIS3 gene or promoter region thereof, preferably in the region as indicated in Table 1A, is preferably determined in a vulvar sample, preferably tissue, cell or cell free DNA sample.

Hypermethylation or methylation status of FRMD4B gene or promoter region thereof, preferably in the region as indicated in Table 1A, is preferably determined in a cervical sample, preferably tissue, cell or cell free DNA sample.

In preferred embodiments, the methylation status of the one or more genes, promoter regions and/or chromosomal regions listed in table 1 or table 1A is compared with a reference. The term “reference” as used herein refers to a sample or value from one or more healthy individuals not suffering from cancer or to a sample or value from one or more individuals suffering from cancer, in particular a gynaecological, or ano-uro-genital, head and neck or cutaneous cancer. Alternatively, a reference refers to a sample or value from one or more individuals that are known to suffer from cancer, in particular a gynaecological or ano-uro-genital cancer. A reference value is preferably a value for methylation of the same gene, promoter region or chromosomal region of which the methylation status is determined. In preferred embodiments, the methylation status is compared with a reference value. A reference sample is preferably the same type of sample as the sample that is used for determining methylation status. I.e. a reference sample is a comparable sample as the sample used for determining methylation status. Similarly, a reference value is preferably a value for methylation The reference can be a reference sample obtained from a single individual. It is, however, preferred, that the reference is a value based on sample from a plurality of individuals, i.e. a plurality of healthy individuals, or a plurality of subjects known to suffer from cancer, preferably the same type of cancer. Said plurality is for instance at least 5 individuals, at least 10 individuals, at least 20 individuals, at least 30 individuals, at least 50 individuals, at least 75 individuals, or at least 100 individuals. Based on a comparison with a reference, in particular a reference sample or reference value, it can be determined whether an individual is suffering from or at risk of suffering from cancer. In preferred embodiments, differential methylation of the one or more genes, promoter regions or chromosomal regions, in particular as compared to a reference value, is indicative of cancer or a predisposition to cancer. In further preferred embodiments, hypermethylation of the one or more genes, promoter regions or chromosomal regions, in particular as compared to a reference value, is indicative of cancer or a predisposition to cancer. The specific reference, in particular reference sample or reference value, that is used in a method or use of the invention, depends on the specific method, and a skilled person is well capable of identifying and using an appropriate reference, reference sample and/or reference value.

Methods to determine or detect the methylation status of genes, promoter sequences or chromosomal regions are known in the art. Detection is performed on nucleic acid, in particular DNA. Suitable methods include the use of nucleic acid primers and/or a probe and restriction endonucleases, in particular methylation specific primers and/or probe for methylation specific PCR and methylation sensitive and/or dependent restriction endonucleases.

Methylation-Specific PCR (MSP), including quantitative methylation-specific PCR (QMSP,) is based on a reaction of bisulfite, e.g. sodium bisulfite, with DNA which converts unmethylated cytosines of to uracil, followed by a sequence-specific PCR. Methylated cytosines will not be converted with this reaction and the sequence-specific primers are designed to overlap the site that show differential methylation in cancer. This allows for the determination of the methylation status of the site as methylated or unmethylated.

As an alternative methylation-sensitive and/or dependent restriction endonucleases can be used to detect methylated CpG dinucleotide motifs. Such endonucleases may either preferentially cleave non-methylated recognition sites relative to methylated recognition sites or preferentially cleavemethylated relative to non-methylated recognition sites, respectively.

Other means for detection of hypermethylation that are reliant on specific sequences can be used, including but not limited to electrophoresis, hybridization, amplification, sequencing (e.g. pyrosequencing of bisulfite treated DNA or TAPS (Tet-assisted pyridine borane sequencing) converted DNA), methyl sensitive southern blotting, methylated DNA immunoprecipitation (MeDIP), ligase chain reaction, chromatography, mass spectrometry and combinations of such techniques.

In preferred embodiments, the methylation status, DNA methylation, in particular hypermethylation, of one or more genes, promoter regions thereof or chromosomal regions listed in table 1 or table 1A is determined or detected using primer and/or probes specific for said one or more genes, promoter regions and/or chromosomal regions. Hence, in preferred embodiments, means for detecting methylation status of a gene, promoter region thereof or chromosomal region as defined herein comprise primers and/or probes specific for said gene, promoter region or chromosomal region.

In preferred embodiments, the methylation status, DNA methylation, in particular hypermethylation, of the one or more genes, promoter sequences or chromosomal regions is determined using methylation specific PCR or quantitative methylation-specific PCR (QMSP).

In preferred embodiments, the methylation status, DNA methylation, in particular hypermethylation, of the one or more genes, promoter sequences or chromosomal regions is determined using methylation specific primers and/or probes.

In preferred embodiments, hypermethylation of one or more genes and/or promoter regions thereof and/or one or more chromosomal regions selected from table 1 indicates a sample of an individual suffering from gynaecological, ano-uro-genital, head and neck or cutaneous cancer or a sample of an individual that is predisposed to a gynaecological, ano-uro-genital, head and neck or cutaneous cancer.

In preferred embodiments, hypermethylation of one or more genes and/or promoter regions thereof and/or one or more chromosomal regions selected from table 1 indicates a sample of an individual suffering from a cervical, vulvar, endometrial, ovarian, fallopian tube, vaginal, colon, colorectal, anal, head and neck, penile, bladder, prostate and cutaneous cancer, more in particular cervical, vulvar, endometrial, ovarian, fallopian tube, head/neck, cutaneous, colorectal and anal cancer and precancerous lesions thereof, more in particular cervical, vulvar, endometrial, ovarian, fallopian tube, head/neck and colorectal cancer, or a sample of an individual that is predisposed to a cervical, vulvar, endometrial, ovarian, fallopian tube, vaginal, colon, colorectal, anal, head and neck, penile, bladder, prostate and cutaneous cancer, more in particular cervical, vulvar, endometrial, ovarian, fallopian tube, head/neck, cutaneous, colorectal and anal cancer and precancerous lesions thereof, more in particular cervical, vulvar, endometrial, ovarian, fallopian tube, head/neck and colorectal cancer.

In preferred embodiments, hypermethylation of METloc009 indicates a sample of an individual suffering from a cervical or vulvar cancer.

In preferred embodiments, hypermethylation of GLIS3 or promoter region indicates a sample of an individual suffering from a vulvar cancer.

In preferred embodiments, hypermethylation of FRMD4B indicates a sample of an individual suffering from a cervical adenocarcinoma.

In preferred embodiments methylation status, DNA methylation, in particular hypermethylation, is determined in the CpG rich promoter and/or sequence of the genes and/or chromosomal regions listed in table 1 or table 1A.

The invention further provides a kit of parts comprising means for the detection of methylation status and/or DNA methylation, in particular hypermethylation, of one or more genes and/or promoter regions thereof and/or one or more chromosomal regions selected from table 1 or table 1A. In preferred embodiments, said means comprise one or more primers and/or probes specific for said one or more genes and/or promoter regions thereof and/or one or more chromosomal regions. In further preferred embodiments, said primers and/or probes are methylation specific primers and/or probes.

In one aspect, the invention provides a kit of parts comprising:
methylation specific PCR primers complementary to the sequence of chromosomal region METloc009 as indicated in table 1A, GLIS3 gene or a promoter region thereof and/or FRMD4B gene or a promoter region thereof, in particular to the sequence of the chromosomal location(s) for METloc009, GLIS3 and/or FRMD4B indicated in table 1A, or to a bisulfite converted sequence thereof, and
a probe specific for the same marker or markers, i.e. chromosomal region METloc009 as indicated in table 1A, GLIS3 gene or a promoter region thereof and/or FRMD4B gene or a promoter region thereof, in particular to the sequence of the same marker or markers, i.e. chromosomal location(s) for METloc009, GLIS3 and/or FRMD4B indicated in table 1A, preferably to a bisulfite converted sequence thereof.

In another aspect, the invention provides a kit of parts comprising:—methylation specific PCR primers complementary to the sequence of one or more genes and/or promoter regions thereof and/or one or more chromosomal regions selected from table 1, in particular to the sequence of the chromosomal location indicated in table 1, or to a bisulfite converted sequence thereof, and
a probe specific for one or more genes and/or promoter regions thereof and/or one or more chromosomal regions selected from table 1, in particular to the sequence of the chromosomal location indicated in table 1, or to a bisulfite converted sequence thereof, preferably to a bisulfite converted sequence thereof.

In another preferred embodiment, a kit of parts of the invention comprises means for detection of methylation status and/or DNA methylation, in particular hypermethylation, of METloc001, METloc002, ARID3C or a promoter region thereof or ARL5C or a promoter region thereof, optionally in combination with means for detection of methylation status and/or DNA methylation, in particular hypermethylation, of one or more other genes and/or promoter regions thereof and/or one or more other chromosomal regions selected from table 1. Said means preferably comprise one or more primers and/or probes specific for at least METloc001, METloc002 or ARID3C or a promoter region thereof. In one preferred embodiment, said means comprise one or more primers and/or probes specific for METloc001, in particular to the sequence of the chromosomal location indicated in table 1. In one preferred embodiment, said means comprise one or more primers and/or probes specific for METloc002, in particular to the sequence of the chromosomal location indicated in table 1. In one preferred embodiment, said means comprise one or more primers and/or probes specific for ARID3C or a promoter region thereof, in particular to the sequence of the chromosomal location indicated in table 1.

In preferred embodiments, a kit of parts of the invention comprises means, preferably primers and/or a probe, specific for at least two genes and/or promoter regions thereof and/or one or more chromosomal regions selected from table 1, in particular specific for a bisulfite converted sequence thereof. Said at least two genes and/or promoter regions thereof and/or one or more chromosomal regions selected from table 1 are preferably selected from METloc001, METloc002, ARID3C or a promoter region thereof and ARL5C or a promoter region thereof. In further preferred embodiments, a kit of parts of the invention comprises means, preferably primers and/or a probe, specific for at least three genes and/or promoter regions thereof and/or one or more chromosomal regions selected from table 1, in particular specific for a bisulfite converted sequence thereof. Said at least three genes and/or promoter regions thereof and/or one or more chromosomal regions selected from table 1 are preferably selected from METloc001, METloc002, ARID3C or a promoter region thereof and ARL5C or a promoter region thereof. In preferred embodiments, said primers and probe are methylation specific primers and a probe. Methylation specific primers and probe are in particular specific for a bisulfite converted sequence of the relevant gene, promoter region and/or chromosomal region.

In some preferred embodiments, a kit of parts of the invention comprises means for detection of methylation status and/or DNA methylation, in particular hypermethylation, of METloc001 and METloc002, in particular of the chromosomal location indicated for these genes in table 1, optionally in combination with means for detection of methylation status and/or DNA methylation, in particular hypermethylation, of one or more other genes and/or promoter regions thereof and/or one or more other chromosomal regions selected from table 1. Said means preferably comprise one or more primers and/or probes specific for METloc001 and METloc002, in particular the chromosomal location indicated in table 1, or to a bisulfite converted sequence thereof, preferably to a bisulfite converted sequence thereof.

In some preferred embodiments, a kit of parts of the invention comprises means for detection of methylation status and/or DNA methylation, in particular hypermethylation, of ARID3C or a promoter region thereof and ARL5C or a promoter regions thereof, in particular of the chromosomal location indicated for these genes in table 1, optionally in combination with means for detection of methylation status and/or DNA methylation, in particular hypermethylation, of one or more other genes and/or promoter regions thereof and/or one or more other chromosomal regions selected from table 1. Said means preferably comprise one or more primers and/or probes specific for ARID3C or a promoter region thereof and ARL5C or a promoter regions thereof or to a bisulfite converted sequence thereof, preferably to a bisulfite converted sequence thereof.

Other preferred kits comprise means, in particular primers and a probe, more in particular methylation specific primers and a probe, for the preferred combinations of genes or a promoter region thereof and/or chromosomal region selected from table 1 indicated herein above.

In preferred embodiments, a kit of parts of the invention comprising means for the detection of methylation status and/or DNA methylation, in particular hypermethylation, of one or more genes and/or promoter regions thereof and/or one or more chromosomal regions selected from table 1 is a kit of part for use in identifying a gynaecological or ano-uro-genital tissue or cell sample or a tonsil, skin, blood, plasma, serum, saliva, serous fluid, urine or stool sample as indicative of cancer or as indicative of a predisposition to cancer.

In other preferred embodiment, the kit of parts of the invention is a kit of parts for use in determining hypermethylation or methylation status of one or more genes and/or promoter regions thereof and/or one or more chromosomal regions selected from table1 in a sample of an individual.

The invention also provides a use of a kit of parts according to the invention comprising means for the detection of methylation status and/or DNA methylation, in particular hypermethylation, of one or more genes and/or promoter regions thereof and/or one or more chromosomal regions selected from table 1 for classifying an individual as suffering from or being at risk of suffering from cancer, in particular a gynaecological, ano-uro-genital, head and neck or cutaneous cancer, as defined herein.

In other preferred embodiments, the kit of parts of the invention is a kit of parts for use in determining hypermethylation or methylation status of chromosomal region METloc009 as indicated in table 1A, GLIS3 gene or a promoter region thereof and/or FRMD4B gene or a promoter region thereof in a sample of an individual. the invention provides a use of such kit of parts according to the invention for classifying an individual as suffering from or being at risk of suffering from cervical cancer or vulvar cancer or precancerous lesion thereof, in particular for:
classifying an individual as suffering from or being at risk of suffering from cervical cancer or vulvar cancer if hypermethylation of METloc009 and/or GLIS3 is determined,
classifying an individual as suffering from or being at risk of suffering from cervical cancer or precancerous lesion thereof if hypermethylation of METloc009 is determined, and/or
classifying an individual as suffering from or being at risk of suffering from cervical carcinoma if hypermethylation of FRMD4B is determined.

The terms “primer” and “probe” are well known in the art. The term “primer” refers to a single-stranded oligonucleotide that is capable of annealing to a target nucleotide sequence allowing a DNA polymerase to attach, thereby serving as a point of initiation of DNA synthesis when placed under conditions in which synthesis of primer extension product which is complementary to a nucleic acid strand is induced, i.e., in the presence of nucleotides and an agent for polymerization such as DNA polymerase and at a suitable temperature and pH. A primers specific for a gene or sequence or a primer pair as used herein refers to one forward and one reverse primer as commonly used in DNA amplification such as in polymerase chain reaction (PCR) amplification. The term “probe” refers to a single-stranded oligonucleotide that will recognize and hybridize to a complementary sequence in a target nucleotide sequence. As used herein, when using primers or a primer pair and a probe specific for a certain gene, promoter region or chromosomal location, the probe hybridizes to the sequence amplified using the primers or primer pair.

The primers and/or probes used in a method of the invention or in a kit of the invention are preferably capable of hybridizing to the gene sequence or promoter region or chromosomal region as listed in table 1 or table 1A or to a bisulfite converted sequence thereof. As detailed herein above, a methylation specific PCR can be performed based on bisulfite modification of the relevant sequences. Hence, in preferred embodiments, said primers and/or probes are methylation specific primers and/or probes.

In preferred embodiments, the primers and/or probes used in a method of the invention or in a kit of the invention are selected from table 14 or table 15 or sequences that are at least 75% identical thereto and able to specifically hybridize to their target sequence, more preferably at least 80%, 85%, 90% or 95% identical thereto and able to specifically hybridize to their target sequence. In some embodiments, at least one of the primers and/or the probe contain at least one locked nucleic acid base.

In preferred embodiments, at least one of the primers or of the primer pair specific for one or more genes and/or promoter regions thereof and/or one or more chromosomal regions selected from table 1 or table 1A or to a bisulfite converted sequence thereof used in a method of the invention present in a kit of the invention comprise a detectable label. In some embodiments both primers comprises a detectable label. Alternatively or additionally, a probe specific for one or more genes and/or promoter regions thereof and/or one or more chromosomal regions selected from table 1 or table 1A used in a method of the invention present in a kit of the invention comprise a detectable label. Suitable detectable labels, both for primers and probes, include radioisotopes, enzymes, and physicochemical reporters, such as fluorescent or chemiluminescent dyes.

In one aspect, the invention provides a method of treating an individual suffering from or being at risk of suffering from a gynaecological, ano-uro-genital, head and neck or cutaneous cancer, the method comprising:
classifying an individual according to a method of the invention;
identifying an individual that is suffering from or at risk of suffering from a gynaecological, ano-uro-genital, head and neck or cutaneous cancer; and
referring of the individual to the appropriate medical specialist, the method further preferably comprising:
identifying the gynaecological, ano-uro-genital, head and neck or cutaneous cancer; and
regular examining of the status and/or monitoring of the cancer or precancerous lesion; and/or
providing treatment of the cancer or precancerous lesion the identified individual.
   Treatment options include as local resection or cryosurgery, laser therapy, hysterectomy, salpingo-oophorectomy, penectomy, transurethral resection, cystectomy, prostatectomy, polypectomy, colectomy, proctectomy, pelvic exenteration, laryngectomy, thyroidectomy, radiation therapy, chemotherapy, hormone therapy, immune therapy, gene therapy and combinations thereof. As will appreciated by the skilled person, the type of treatment depends on the cancer or precancerous lesion identified.

In another aspect, the invention provides a method of treating an individual suffering from or being at risk of suffering from cervical cancer or vulvar cancer or precancerous lesion thereof, the method comprising:
classifying an individual with a method according to the invention;
identifying an individual that is suffering from or at risk of suffering from cervical cancer or vulvar cancer or precancerous lesion thereof; and
referring of the individual to the appropriate medical specialist,
   the method further preferably comprising:
identifying the cervical cancer or vulvar cancer or precancerous lesion thereof; and
regular examining of the status and/or monitoring of the cancer or precancerous lesion; and/or
providing treatment of the cancer or precancerous lesion the identified individual, such as local resection or cryosurgery, laser therapy, hysterectomy, radiation therapy, chemotherapy, hormone therapy, immune therapy and combinations thereof.

In preferred embodiments of these methods detection of hypermethylation in chromosomal region METloc009 as indicated in table 1A and/or GLIS3 gene or a promoter region thereof indicates the presence of vulvar cancer or a precancerous lesion, in particular vulvar cancer.

In further preferred embodiments of these methods, detection of hypermethylation in chromosomal region METloc009 as indicated in table 1A indicates the presence of cervical cancer or a precancerous lesion, in particular cervical cancer.

In further preferred embodiments of these methods, detection of hypermethylation in FRMD4B gene or a promoter region thereof indicates the presence of cervical adenocarcinoma.

As used herein “identifying” the cancer means establishing the type of cancer. In preferred embodiments it means identifying the cancer as cervical, vulvar, endometrial, ovarian, fallopian tube, vaginal, colon, colorectal, anal, head and neck, penile, bladder, prostate or cutaneous cancer, more in particular cervical, vulvar, endometrial, ovarian, fallopian tube, head/neck, cutaneous, colorectal and anal cancer and precancerous lesions thereof, more in particular cervical, vulvar, endometrial, ovarian, fallopian tube, head/neck or colorectal cancer. In some embodiments it means identifying a HPV induced cancer as cervical, vulvar, vaginal, anal, head and neck, penile, and cutaneous cancer and/or identifying a non-HPV induced gynaecological, ano-uro-genital, head and neck or cutaneous cancer as endometrial, ovarian, colon, colorectal, bladder, prostate, fallopian tube cancer. The type of cancer can be determined using known methods in the art, such as cytology or known cancer-specific markers.

In another aspect, the invention provides a method for assigning treatment to an individual of whom hypermethylation of in a gynaecological or ano-uro-genital tissue or cell sample or a tonsil, skin, blood, plasma, serum, saliva, serous fluid, urine or stool sample is determined.

In preferred embodiments, treating an individual or treatment of an individual is tailored to the specific type of gynaecological, ano-uro-genital, head and neck or cutaneous cancer. Hence, in preferred embodiments a method of the invention comprises identifying the gynaecological, ano-uro-genital, head and neck or cutaneous cancer, in particular a cancer as defined herein.

In some embodiments, in particular if the individual is identified as being at risk of suffering from a gynaecological, ano-uro-genital, head and neck or cutaneous cancer, in particular a cervical, vulvar, endometrial, ovarian, fallopian tube, vaginal, colon, colorectal, anal, head and neck, penile, bladder, prostate or cutaneous cancer, more in particular cervical, vulvar, endometrial, ovarian, fallopian tube, head/neck, cutaneous, colorectal and anal cancer and precancerous lesions thereof, more in particular cervical, vulvar, endometrial, ovarian, fallopian tube, head/neck or colorectal cancer, with a marker, in particular multi-cancer marker, of the invention, treatment comprises regular examination of the status and/or monitoring of the cancer or precancerous lesion. Such regular examination refers preferably to periodic examining, e.g. annually, bi-annually or three-monthly, examining of the status and/or monitoring of the cancer or precancerous lesion, such as by cancer specific diagnostics, including e.g. cytology.

In some embodiments, treatment comprises examination of the status of the cancer and/or monitoring the cancer or precancerous lesion by referral to the appropriate medical specialist after a positive test outcome with a marker of the invention. The choice of medical specialist is dependent on sample type tested (i.e. gynaecological or ano-uro-genital tissue or cell sample or a tonsil, skin, blood, plasma, serum, saliva, serous fluid, urine or stool sample). Based on said examination it can be decided to therapeutically treat the cancer or precancerous lesion. Treatment options include as local resection or cryosurgery, laser therapy, hysterectomy, salpingo-oophorectomy, penectomy, transurethral resection, cystectomy, prostatectomy, polypectomy, colectomy, proctectomy, pelvic exenteration, laryngectomy, thyroidectomy, radiation therapy, chemotherapy, hormone therapy, immune therapy and combinations thereof. As will appreciated by the skilled person, the type of treatment depends on the cancer or precancerous lesion identified.

Referral to a gynaecologist should be initiated after a positive test outcome from a gynaecological or genital tissue or cell sample for further cancer staging or risk to cancer assessment regarding cervical cancer, vulvar cancer, endometrial cancer, ovarian cancer, fallopian tube cancer, vaginal cancer or precancerous lesion thereof. Gynaecological assessment of these cancers and associated precancerous lesions, and positive identification of such cancer or precancerous lesion, may lead to local resection or cryosurgery or laser therapy or hysterectomy or salpingo-oophorectomy in combination with or by itself treated with radiation therapy and/or chemotherapy and/or hormone therapy and/or immune therapy, in particular at a stage of the cancer or precancerous lesion when such treatment is appropriate. Such determination is preferably based on the regular examination of the status and/or monitoring of the cancer or precancerous lesion.

Referral to an urologist should be initiated after a positive test outcome from an uro-genital tissue or cell sample or urine sample for further cancer staging or risk to cancer assessment regarding penile cancer, bladder cancer, prostate cancer or precancerous lesion thereof. Urological assessment of these cancers and associated precancerous lesions, and positive identification of such cancer or precancerous lesion, may lead to local resection or cryosurgery or laser therapy or penectomy or transurethral resection or cystectomy or prostatectomy in combination with or by itself treated with radiation therapy and/or chemotherapy and/or hormone therapy and/or immune therapy, in particular at a stage of the cancer or precancerous lesion when such treatment is appropriate. Such determination is preferably based on the regular examination of the status and/or monitoring of the cancer or precancerous lesion.

Referral to a specialist in Internal Medicine should be initiated after a positive test outcome from a tonsil, skin, blood, plasma, serum or stool sample for further cancer staging or risk to cancer assessment regarding colon cancer, colorectal cancer, anal cancer, cutaneous cancer or precancerous lesion thereof. Assessment by specialist in Internal Medicine of these cancers and associated precancerous lesions, and positive identification of such cancer or precancerous lesion, may lead to local resection or polypectomy or colectomy or proctectomy or pelvic exenteration in combination with or by itself treated with radiation therapy and/or chemotherapy and/or hormone therapy and/or immune therapy, in particular at a stage of the cancer or precancerous lesion when such treatment is appropriate. Such determination is preferably based on the regular examination of the status and/or monitoring of the cancer or precancerous lesion.

Referral to an otolaryngologist (ENT, ears nose throat) should be initiated after a positive test outcome from a saliva sample for further cancer staging or risk to cancer assessment regarding head and neck cancer or precancerous lesion thereof. Assessment by an otolaryngologust (ENT, ears nose throat) of above-mentioned cancers and associated precancerous lesions, and positive identification of such cancer or precancerous lesion, may lead to local resection or laser surgery or laryngectomy or thyroidectomy in combination with or by itself treated with radiation therapy and/or chemotherapy and/or hormone therapy and/or immune therapy, in particular at a stage of the cancer or precancerous lesion when such treatment is appropriate. Such determination is preferably based on the regular examination of the status and/or monitoring of the cancer or precancerous lesion.

In addition, referral to a General Practitioner (GP) can be initiated after a positive test outcome in a screening setting where testing was not initiated by a specific medical specialist. This may apply to testing in a gynaecological or ano-uro-genital tissue or cell sample or a tonsil, skin, blood, plasma, serum, saliva, serous fluid, urine or stool sample. In this situation cancer staging or risk to cancer assessment should focus on cervical cancer, vulvar cancer, endometrial cancer, ovarian cancer, fallopian tube cancer, vaginal cancer, induced colon, colorectal, anal cancer, head and neck cancer, penile cancer or precancerous lesion thereof, in particular HPV induced cervical cancer, vulvar cancer, endometrial cancer, ovarian cancer, fallopian tube cancer, vaginal cancer, induced colon, colorectal, anal cancer, head and neck cancer, penile cancer or precancerous lesion thereof.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: IGV plot showing a different methylated region (DMR) with higher abundance in several distinct cancer samples over two different control type samples illustrating regional pan markers.

FIG. 2: Receiver operating characteristic (ROC) curve analysis and area under the curve (AUC) of MeD-seq read counts for 11 general markers (METloc001, METloc002, METloc003, METloc004, METloc005, METloc006, METloc007, METloc008, ARID3C, ARL5C and TPTEP1 for detecting cervical, vulvar, endometrial, fallopian tube and ovarian cancer in whole tissue (LCM) sections.

FIG. 3: Boxplot comparison of the MeD-seq read counts for eleven general markers for detecting vulvar, cervical, endometrial, fallopian tube and ovarian cancer in LCM samples.

FIG. 4: Receiver operating characteristic (ROC) curve analysis and area under the curve (AUC) of MeD-seq read counts for METloc009; cervical and vulvar cancer, GLIS3; vulvar cancer and FRMD4B; cervical adenocarcinoma. Results for vulvar, cervical, endometrial, fallopian tube and ovarian cancer LCM samples.

FIG. 5: Boxplot comparison of the MeD-seq read counts for METloc009; cervix and vulvar cancer, GLIS3; vulvar cancer and FRMD4B; cervical adenocarcinoma. Results for vulvar, cervical, endometrial, fallopian tube and ovarian cancer in LCM samples.

FIG. 6: Receiver operating characteristic (ROC) curve analysis and area under the curve (AUC) of methylation ratios for METloc001, METloc002, ARID3C and ARL5C for detecting vulvar, cervical, endometrial, fallopian tube and ovarian cancer in whole tissue sections.

FIG. 7: Boxplot comparison of the qMSP results of METloc001, METloc002, ARID3C and ARL5C in formalin fixed paraffin embedded (FFPE) normal, precancer and cancer tissues of the vulva. * p<0.05, ** p<0.01, *** p<0.001, Ref: reference, ns: not significant, na: not applicable

FIG. 8: Boxplot comparison of the quantitative methylation-specific PCR (qMSP) results of METloc001, METloc002, ARID3C and ARL5C in formalin fixed paraffin embedded (FFPE) normal, precancer and cancer tissues of the cervix. * p<0.05, ** p<0.01, *** p<0.001, Ref: reference, ns: not significant, na: not applicable

FIG. 9: Boxplot comparison of the qMSP results of METloc001, METloc002, ARID3C and ARL5C in formalin fixed paraffin embedded (FFPE) normal and cancer tissues of the endometrium. * p<0.05, ** p<0.01, *** p<0.001, Ref: reference, ns: not significant, na: not applicable

FIG. 10: Boxplot comparison of the qMSP results of METloc001, METloc002, ARID3C and ARL5C in formalin fixed paraffin embedded (FFPE) normal and cancer tissues of the fallopian tube. * p<0.05, ** p<0.01, *** p<0.001, Ref: reference, ns: not significant, na: not applicable

FIG. 11: Boxplot comparison of the qMSP results of METloc001, METloc002, ARID3C and ARL5C in formalin fixed paraffin embedded (FFPE) normal and cancer tissues of the ovaries. * p<0.05, ** p<0.01, *** p<0.001, Ref: reference, ns: not significant, na: not applicable

FIG. 12: Receiver operating characteristic (ROC) curve analysis and area under the curve (AUC) of methylation ratios for METloc001, METloc002, ARID3C and ARL5C for detecting tonsil cancer in whole tissue sections.

FIG. 13: Boxplot comparison of the qMSP results of METloc001, METloc002, ARID3C and ARL5C in formalin fixed paraffin embedded (FFPE) normal and cancer tissues of the tonsils. * p<0.05, ** p<0.01, *** p<0.001, Ref: reference, ns: not significant, na: not applicable

FIG. 14: Receiver operating characteristic (ROC) curve analysis and area under the curve (AUC) of methylation ratios for METloc001, METloc002, ARID3C and ARL5C for detecting colorectal and anal cancers in whole tissue sections.

FIG. 15: Boxplot comparison of the qMSP results of METloc001, METloc002, ARID3C and ARL5C in formalin fixed paraffin embedded (FFPE) normal and cancer tissues of colon and rectum. * p<0.05, ** p<0.01, *** p<0.001, Ref: reference, ns: not significant, na: not applicable

FIG. 16: Boxplot comparison of the qMSP results of METloc001, METloc002, ARID3C and ARL5C in formalin fixed paraffin embedded (FFPE) normal and cancer tissues of the anal tissue. * p<0.05, ** p<0.01, *** p<0.001, Ref: reference, ns: not significant, na: not applicable

FIG. 17: Receiver operating characteristic (ROC) curve analysis and area under the curve (AUC) of methylation ratios for METloc001 and METloc002 for detecting skin cancer in whole tissue sections.

FIG. 18: Boxplot comparison of the qMSP results of METloc001 and METloc002 in formalin fixed paraffin embedded (FFPE) normal, precancer (Actinic Keratosis (AK), Bowen's disease (MB; Morbus Bowen)) and cancer of skin tissue. * p<0.05, ** p<0.01, *** p<0.001, Ref: reference, ns: not significant, na: not applicable

FIG. 19: Receiver operating characteristic (ROC) curve analysis and area under the curve (AUC) of methylation ratios for METloc009; cervical and vulvar cancer, GLIS3; vulvar cancer and FRMD4B; cervical adenocarcinoma. Results for vulvar and cervical cancer in whole tissue sections.

FIG. 20: Boxplot comparison of the qMSP results of METloc009, GLIS3 and FRMD4B in formalin fixed paraffin embedded (FFPE) normal, precancer and cancer tissues of vulva and cervix (SCC-HPV negative, SCC+HPV positive). * p<0.05, ** p<0.01, *** p<0.001, Ref: reference, ns: not significant, na: not applicable

FIG. 21: Receiver operating characteristic (ROC) curve analysis and area under the curve (AUC) of methylation ratios for METloc009 detecting colorectal and anal cancer in whole tissue sections.

FIG. 22: Boxplot comparison of the qMSP results of METloc009 in formalin fixed paraffin embedded (FFPE) normal, precancer and cancer of the colorectal and anal tissues. * p<0.05, ** p<0.01, *** p<0.001, Ref: reference, ns: not significant, na: not applicable

FIG. 23: Receiver operating characteristic (ROC) curve analysis and area under the curve (AUC) of methylation ratios for METloc001, METloc002, ARID3C and ARL5C for detecting CIN3+ in physician taken cytology samples.

FIG. 24: Boxplot comparison of the qMSP results of METloc001, METloc002, ARID3C and ARL5C methylation in physician-taken cytology samples of normal, CIN1, CIN2, CIN3, CIN3+ and cervix cancer patients. * p<0.05, ** p<0.01, *** p<0.001, Ref: reference, ns: not significant, na: not applicable

### EXAMPLES

### Materials and Methods

To identify epigenetic changes that could play a role in the development of precancer and cancer, DNA methylation profiles of human cancers and corresponding normal tissues were generated using the MeD-seq assay. For this. laser capture microdissection (LCM) was used for target cell enrichment. After processing of the NGS data and running of python scripts, marker discovery lists were generated. By combining the marker discovery lists and the visualized methylation profiles, the most promising candidate hypermethylated epigenetic markers could be identified. The MeD-seq read counts were used to generate ROC curve and boxplots. Quantitative methylation-specific PCRs (qMSPs) were developed for these markers and the potential value of the DNA methylation of these markers as markers for the detection of cancer was evaluated by testing samples with and without (pre)cancer DNA. The methylation data were used to generate ROC curve and boxplots.

### Laser Capture Microdissection (LCM) for Target Cell Enrichment

For good-quality genome-wide DNA methylation input of a purest possible population of target cells is required. LCM is an accurate method for isolating these specific target cells out of complex heterogeneous tissue. For LCM, sections of FFPE tissue were cut and mounted on membranes fixed on glass slides. Next, the sections were dewaxed, dehydrated and stained. After digital imaging of the stained slides, regions of interest were annotated by a pathologist. The annotated regions were cut by a laser beam using the Leica Laser Microdissection system and collected in tubes. After the extraction of genomic DNA from the microdissected tissues by proteinase K, the concentrations of the extracted DNA were determined, and the samples were ready for MeD-seq analysis.

### MeD-Seq Assay

MeD-seq assays were essentially performed as previously described [Boers R, Boers J et al. Genome Res. 2018 January; 28(1):88-99. doi:10.1101/gr.222885.117]. In short, 8 μl genomic DNA (input ranged from 8 to 50 ng) from selected tissues were digested with LpnPI (New England Biolabs, Ipswich, MA) yielding 32 bp fragments around the fully methylated recognition site containing a CpG. Samples were prepped for sequencing using the ThruPLEX DNA-seq 96D kit (Rubicon Genomics, Takara Bio Europe, Saint-Germain-en-Laye, France) and purified on a Pippin HT system with 3% agarose gel cassettes (Sage Science, Beverly, MA). Libraries were multiplexed and sequenced until ~20 million reads on an Illumina HiSeq 2500 for 50 bp single reads according to the manufacturer's instructions (Illumina, San Diego, CA).

### Processing of NGS Data

For sorting the obtained sequenced reads into separate files for each sample in a sequenced run the dual indexed samples were demultiplexed using bcl2fastq software (Illumina). Subsequent data processing was carried out using specifically created scripts in Python, which include a trimming step to remove the Illumina adapters and a filtering step based on LpnPI restriction site occurrence between 13 and 17 bp from the 5′ or 3′ end of the read, after which DNA fragments which were methylated remain. Reads passing the filter were mapped to the human genome (hg38) using Bowtie 2 [version 2.3.3, Langmead B, Salzberg S. Fast gapped-read alignment with Bowtie 2. Nature Methods. 2012, 9:357-359]. Using all unambiguously mapped reads, count scores were assigned to each individual LpnPI site in the genome.

### Marker Discovery Lists

Using an in house developed bioinformatics pipeline Mimir chromosomal regions that are differentially methylated are found and ranked. The following python packages are used; NumPy (1.19.1) scikit-learn (0.23.2) and SciPy (1.5.2). All methylation samples from MeD-seq are loaded in and subdivided in control samples and cancer samples. Using a sliding window the whole human genome is scanned for Differentially Methylated Regions (DMR). Regions that have a Mann-Whitney U test p-value above Bonferroni corrected 0.20 are discarded. The DMR that do pass the filter are ranked using AUC score.

### Visualisation of Methylation Profiles

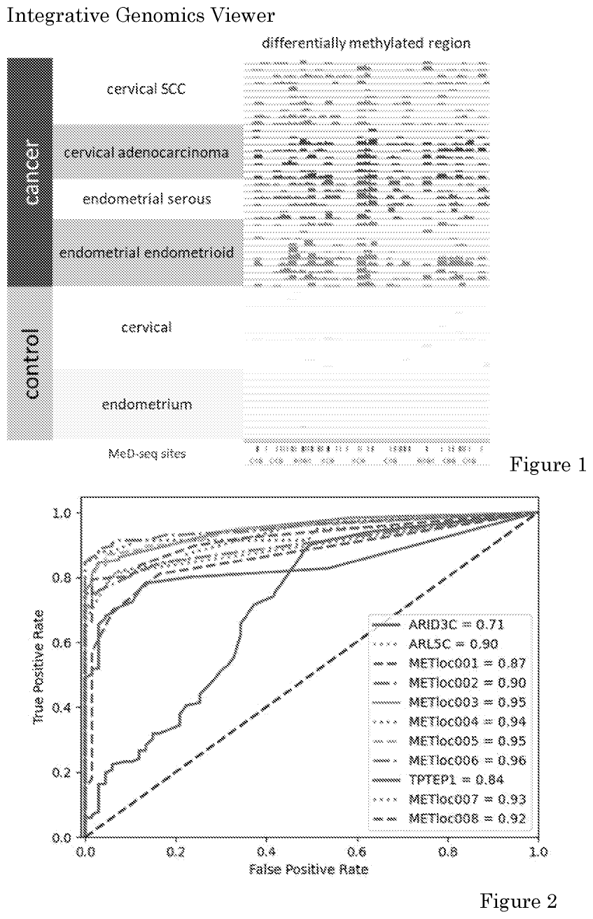

Comparison of cancer and control methylation profiles by an in house developed bioinformatics pipeline Mimir facilitates the identification of specific or general Differentially Methylated Regions (DMR) in a genome wide fashion. However, not all DMR's are suitable to create powerful cancer detection markers. For this, DNA methylation tracks figures were generated using Integrative Genomics Viewer (IGV). By loading in the bigwig files of MeD-seq per sample into IGV, any region on the human genome was visualized showing the MeD-seq methylation per sample (FIG. 1).

### Identification of Candidate Hypermethylated Epigenetic Markers

To determine if a DMR is discriminating enough and suitable to create a powerful marker, we use visualizations in Integrative Genomics Viewer (IGV) using hg38 as reference, to select our top DMR's. These are shown in table 1. After identification of our top DMR's, a selection was made of DMR's that meet all the technical criteria for optimal designing a quantitative methylation-specific PCR (qMSP) able to distinguish samples with tumor DNA from samples without tumor DNA. The qMSP assay is performed on bisulfite converted DNA and requires primer and probe design on the bisulfite-converted sequence of the selected DMR region. Bisulfite-conversion changes unmethylated cytosines to uracil, while methylated cytosines in CpG dinucleotides remain unchanged, allowing methylated DNA to be distinguished from unmethylated DNA. Multiple primers/probe sets covering the most important methylated CpG's in the selected DMR are designed. For selection of the most optimal primers/probe set, bisulfite-converted fully methylated, bisulfite-converted fully unmethylated DNA, non-converted DNA and no template controls are tested.

DNA Methylation Analysis Using Multiplex Quantitative Methylation-Specific PCR (qMSP)

For qMSP analysis, proteinase K digestion was done on sections of FFPE samples. Because these samples generally yield highly degraded DNA, the presence of enough amplifiable human DNA of sufficient quality and the absence of PCR inhibitors were determined using an inhouse DNA QC qPCR. The isolated DNA was bisulfite-converted using the EZ DNA Methylation kit (Zymo Research, Orange, CA, USA) according to the manufacturer's instructions, converting unmethylated cytosines into uracils while methylated cytosines remain unchanged. In brief, the qMSP reaction for the amplification of methylated bisulfite-converted DNA was performed in a final reaction volume of 12.5 μl, containing designed primers/probe sets and 25 ng input of bisulfite-converted DNA in a PCR Master Mix. Bisulfite-converted sample DNA was analyzed for methylation markers using a multiplex quantitative methylation-specific PCR (qMSP) assay, targeting selected genes and the reference gene, 6-actin (ACTB). The qMSP was performed under the following conditions: 95.0° C. for 5 minutes followed by 45 cycles 95.0° C. for 15 seconds and 63.0° C. for 50 seconds. Fluorescence data were collected at the end of each annealing/extension step for determination of the Cq values. For each sample, the Cq values of the marker reactions were normalized for DNA concentration using the ACTB Cq value. The multiplex was performed on the CFX96 7 Real-Time PCR System using in vitro enzymatically methylated human genomic DNA as calibrator. Cq values were measured at fixed thresholds for fluorescence. For each sample, the Cq values of the marker reactions were normalized for DNA concentration using the ACTB Cq value and the ΔΔCq ratio (2^{-ΔΔCq}×100) was calculated by comparing the marker Cq values to the Cq values of ACTB and calibrator. Primers and probes for this multiplex qMSP are shown in Table 14 and Table 15

### ROC Curve and Boxplots

To evaluate methylation levels, boxplots were computed from the log 2-transformed ΔΔCq ratios of the markers. Using a custom python script receiver operating characteristic (ROC) curves and area under the ROC Curve (AUC) are calculated and visualized using the python packages SciPy (1.5.2) and Matplotlib (3.3.1). Boxplots are generated with python packages Matplotlib (3.3.1) and seaborn (0.11.0).

### Results

### Example A. Identification of Differentially Methylated Regions

Table 1B shows DMR's that were identified in the MeD-seq assays as discriminating enough and suitable to create a powerful multi-cancer marker. These DMR's were found within three known genes and their promoter region (ARL3C, ARID5C and TPTEP1) and eight chromosomal regions that are not linked to a known gene (indicated herein as METloc001 to METloc008).

Table 2 shows the sensitivity, specificity, positive and negative predictive values, accuracy and Matthews correlation coefficient of the MeD-seq read counts for these general markers for detecting a cancer selected from vulvar, cervical, endometrial, fallopian tube and ovarian cancer in LCM samples.

The chromosomal locations in which the differential methylation was found is indicated in table 1B for each of the markers. The METloc001 region, METloc002 region, ARL3C and ARID5C were selected for further characterization.

TABLE 2
Classification statistics of MeD-seq read counts for eleven
general markers for detecting a cancer selected from vulvar,
cervical, endometrial, fallopian tube and ovarian cancer
in LCM samples. Sensitivity(SEN), specificity(SPC), positive
and negative predictive values(PPV, NPV), accuracy(ACC)
and Matthews correlation coefficient (MCC).

| Marker | SEN | SPC | PPV | NPV | ACC | MCC |
|---|---|---|---|---|---|---|
| METloc001 | 0.69 | 0.94 | 0.95 | 0.64 | 0.78 | 0.6219 |
| METloc002 | 0.76 | 0.97 | 0.98 | 0.7 | 0.84 | 0.7055 |
| ARID3C | 0.75 | 0.58 | 0.76 | 0.57 | 0.69 | 0.4279 |
| ARL5C | 0.66 | 1 | 1 | 0.63 | 0.79 | 0.6428 |
| METloc003 | 0.81 | 0.99 | 0.99 | 0.75 | 0.87 | 0.7668 |
| METloc004 | 0.86 | 0.97 | 0.98 | 0.8 | 0.9 | 0.8089 |
| METloc005 | 0.87 | 0.96 | 0.97 | 0.81 | 0.9 | 0.8067 |
| METloc006 | 0.89 | 0.97 | 0.98 | 0.83 | 0.92 | 0.8373 |
| TPTEP1 | 0.76 | 0.88 | 0.92 | 0.68 | 0.8 | 0.64 |
| METloc007 | 0.75 | 1 | 1 | 0.7 | 0.84 | 0.7199 |
| METloc008 | 0.79 | 0.99 | 0.99 | 0.73 | 0.86 | 0.7497 |

TABLE 1B
Differentially methylated regions (DMR's) that were identified in the MeD-seq assays.

| | | gene/ | Chromosomal | | | | |
|---|---|---|---|---|---|---|---|
| no | chromosome | region | location (hg38) | Ensembl ID | p-value | fold_change | AUC |
| 1 | chr3 | METloc002 | chr3: 147384749- | | 2.19388E−16 | 38.5775 | 0.9066 |
| | | | 147385097 | | | | |
| 2 | chr1 | METloc001 | chr1: 145475713- | | 5.49025E−17 | 43.512 | 0.89978 |
| | | | 145476399 | | | | |
| 3 | chr9 | ARID3C | chr9: 34623552- | ENSG00000205143 | 4.94517E−09 | 5.27 | 0.784 |
| | | | 34624056 | | | | |
| 4 | chr17 | ARL5C | chr17: 39165088- | ENSG00000141748 | 6.47365E−15 | 68.894 | 0.88922 |
| | | | 39165444 | | | | |
| 5 | chr1 | METloc003 | chr1: 148048856- | | 7.07972E−16 | 42.12333333 | 0.891866667 |
| | | | 148049542 | | | | |
| 6 | chr13 | METloc004 | chr13: 112056904- | | 1.66053E−15 | 41.07 | 0.876714286 |
| | | | 112057851 | | | | |
| 7 | chr7 | METloc005 | chr7: 101302849- | | 8.00582E−16 | 19.8325 | 0.910675 |
| | | | 101303191 | | | | |
| 8 | chr7 | METloc006 | chr7: 35261398- | | 2.99571E−15 | 20.306 | 0.88207 |
| | | | 35262258 | | | | |
| 9 | chr22 | TPTEP1 | chr22: 16601841- | ENSG00000100181 | 2.41102E−15 | 100.62 | 0.86915 |
| | | | 16602129 | | | | |
| 10 | chr1 | METloc007 | chr1: 50415661- | | 8.20676E−16 | 40.97666667 | 0.881733333 |
| | | | 50416003 | | | | |
| 11 | chr7 | METloc008 | chr7: 35257718- | | 7.20169E−16 | 40.67 | 0.881866667 |
| | | | 35258013 | | | | |

### Example 1. METloc001-METloc008, ARL5C, ARID3C and TPTEP1 as General Methylation Markers

H&E stainings of tumor and normal tissue sections were reviewed by a pathologist to confirm correct classification and to annotate tumor and corresponding normal tissue regions on the sections for LCM. The annotated regions were then laser capture microdissected for target cell enrichment. The genomic DNA from the microdissected tissues was extracted by proteinase K, and the extracted DNA was used for MeD-seq analysis.

By comparing methylation profiles of vulvar, cervical, endometrial, fallopian tube and ovarian carcinoma with the methylation profiles of corresponding normal tissue (see FIG. 1), we identified DMR's that displayed higher levels of methylation in the carcinoma compared to the levels of methylation in the corresponding normal tissues. The DMR's in the regions indicated in table 1B were promising to be used for epigenetic marker development.

The usability of the methylation markers for distinguishing (pre)cancerous tissue from normal tissue was investigated by testing a series of biopsies, including vulvar, cervical, endometrial, fallopian tube and ovarian carcinoma and corresponding normal tissue, in a multiplex qMSP. Primers and probes for this multiplex qMSP are shown in Table 14.

TABLE 14
Primer and probe sequences (5′-3′) used for qMSP analysis. Locked
nucleic acid bases are preceded by a plus sign (+); Probe with MGB
incorporate a 5′ fluorescent dye and a minor groove binder (MGB)
conjugated to the 3′ non-fluorescent quencher of the probe.

| Oligo | Sequence |
|---|---|
| ARID3C 1 Forward primer | TTTCGG+T+CGTTTTTATTTTA+C |
| ARID3C Reverse primer | CCCAAACCGAACCCGTA |
| ARID3C Probe with MGB | CGCCCCTAACCTC |
| ARL5C Forward primer | GGGATTTTTGTTTATCGTATCG+C |
| ARL5C Reverse primer | TCCAC+CGTCCCGA |
| ARL5C Probe with MGB | AACAAATATCGAAACTAAATAA |
| ACTB Forward primer | TGGTGATGGAGGAGGTTTAGTAAGT |
| ACTB Reverse primer | AACCAATAAAACCTACTCCTCCCTTAA |
| ACTB Probe | ACCACCACCCAACACACAATAACAAACACA |
| METloc001 Forward primer | GTTTGTGTTTTTTTTGTTTAGTCGTATTC |
| METloc001 Reverse primer | TAATAAAACCCCGATACCGAACG |
| METloc001 Probe with MGB | TTTTCGTTGTTTTTTTCGGATT |
| METloc002 Forward primer | TTGTTTTTTAAAGAGGTCGGGTC |
| METloc002 Reverse primer | TTCCTCCGTCTCGCGAAC |
| METloc002 Probe with MGB | AATTAAAAAAACCTCTCCTCG |

The ROC curve analysis of the MeD-seq was designed on basis of log 2-transformed MeD-seq read counts determined in cancerous tissues (n=116) and corresponding normal tissues (n=67) (See FIG. 2).

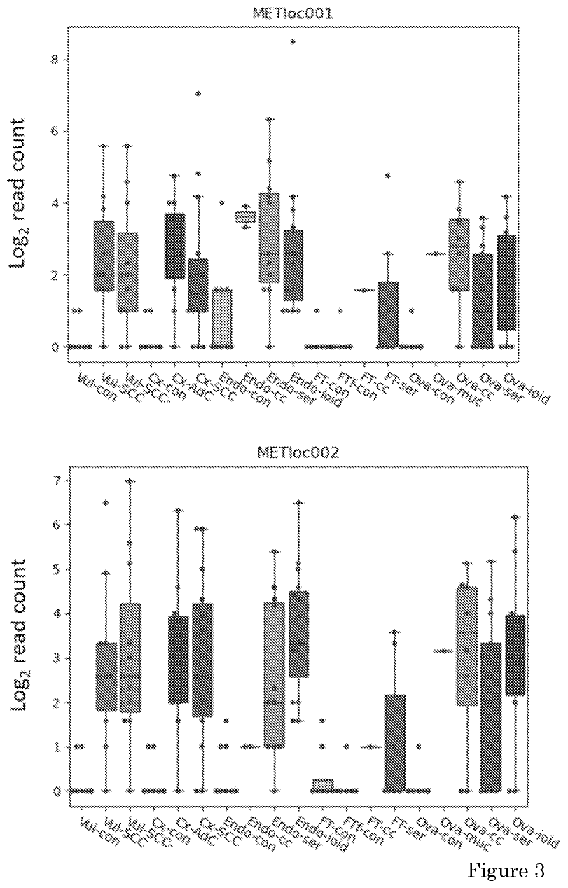

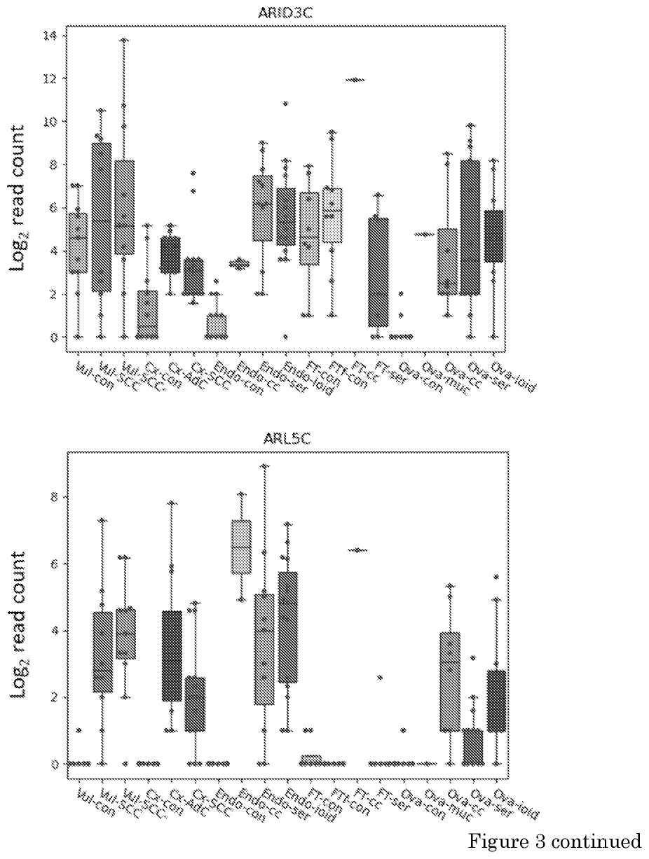

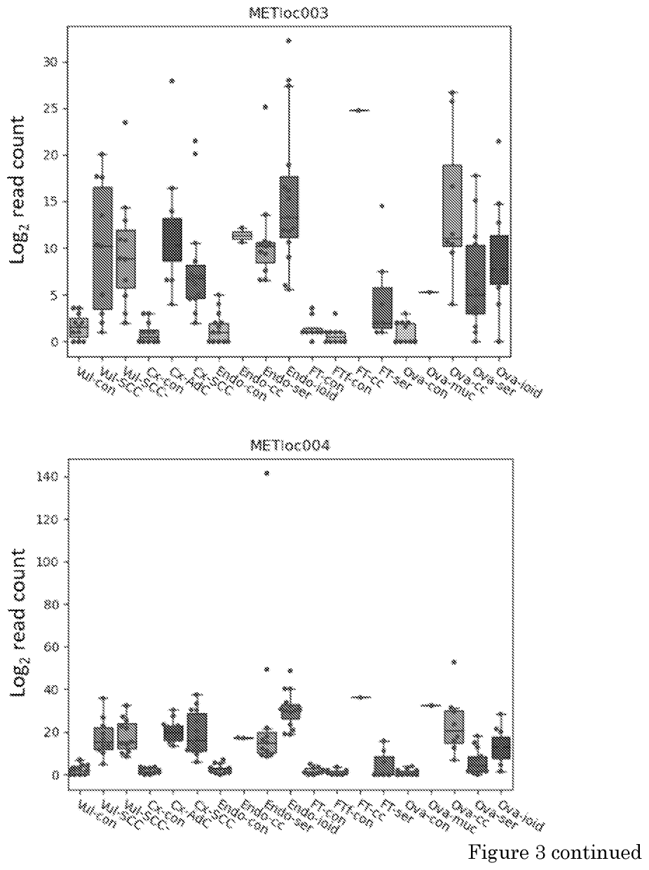

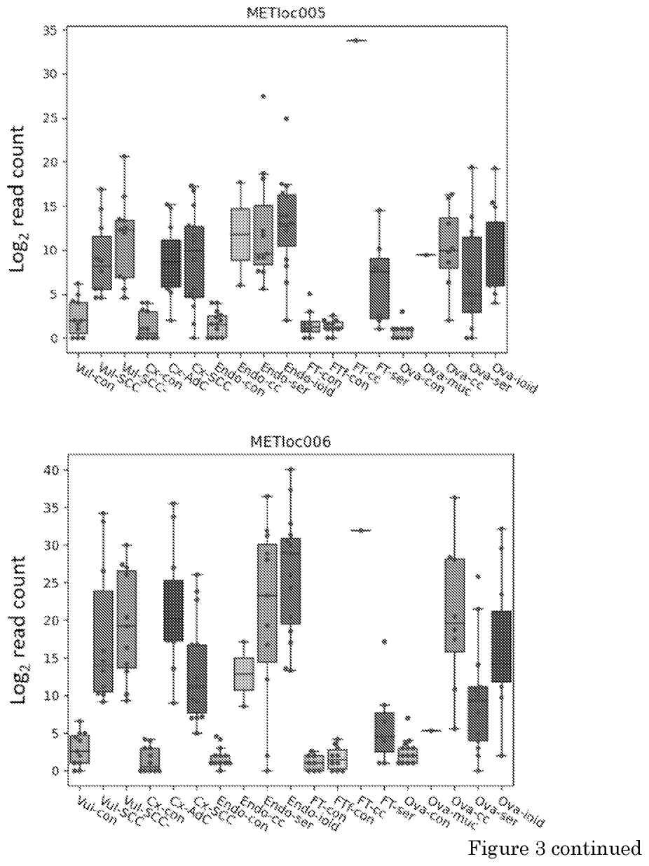

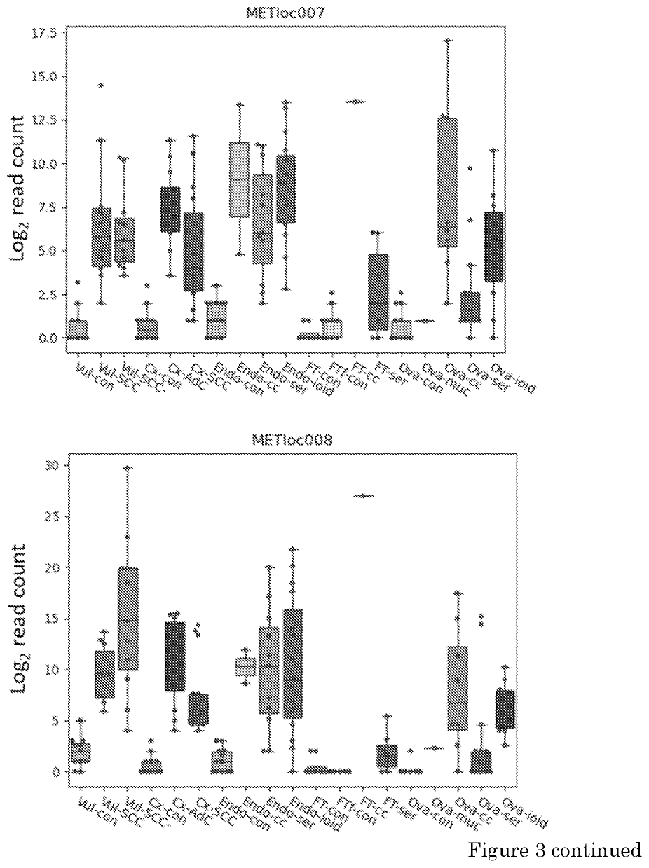

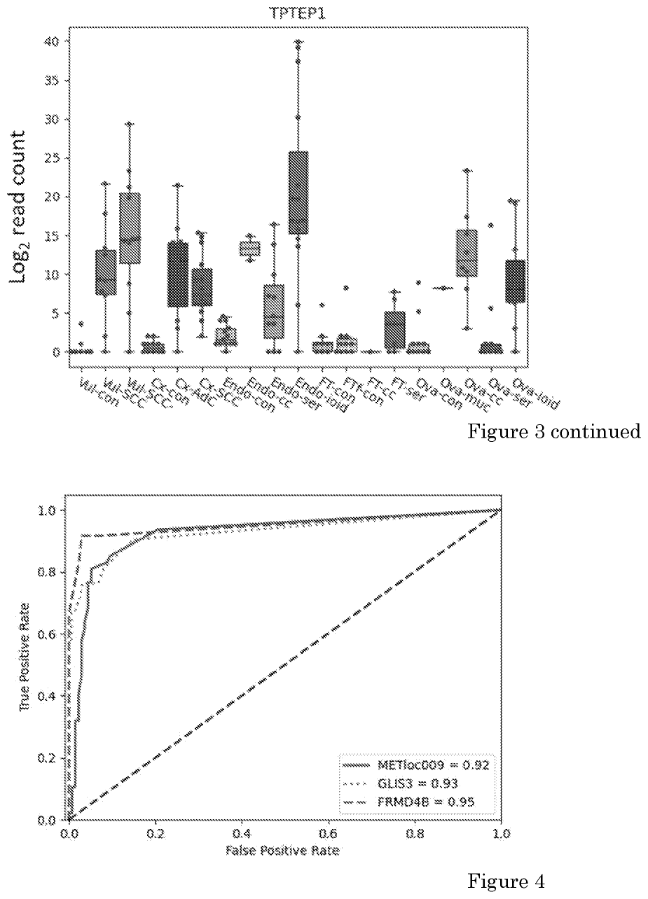

FIG. 3 shows the comparison of the MeD-seq read counts for eleven general markers for detecting vulvar, cervical, endometrial, fallopian tube and ovarian cancer in LCM samples.

ARL5C and ARID3C methylation was determined in cervical normal, precancer and cancer tissues (Example 2), vulvar normal, precancer and cancer tissues (Example 3), endometrial normal and cancer tissues (Example 4), ovarian normal and cancer tissues (Example 5), fallopian tube biopsies (Example 6), head/neck biopsies (Example 7), colorectal and anal biopsies (Example 8), and physician taken cytological samples (Example 10).

METloc001 and METloc002 methylation was determined in cervical normal, precancer and cancer tissues (Example 2A), vulvar normal, precancer and cancer tissues (Example 3A), endometrial normal and cancer tissues (Example 4A), ovarian normal and cancer tissues (Example 5A), fallopian tube biopsies (Example 6A), head/neck biopsies (Example 7A), and colorectal and anal biopsies (Example 8A), skin cancer (Example 9) and physician taken cytological samples (Example 10A).

FIG. 6 shows the receiver operating characteristic (ROC) curve analysis and area under the curve (AUC) of methylation ratios for METloc001, METloc002, ARID3C and ARL5C for detecting vulvar, cervical, endometrial, fallopian tube and ovarian cancer in whole tissue sections. Tables 4 and 5 show the sensitivity, specificity, positive and negative predictive values, accuracy and Matthews correlation coefficient for the individual markers METloc001, METloc002, ARID3C and ARL5C (table 4) and combinations of markers (table 5).

TABLE 4
Classification statistics for METloc001, METloc002, ARID3C
and ARL5C for detecting presence of vulvar, cervical,
endometrial, fallopian tube or ovarian cancer in whole
tissue sections. Sensitivity(SEN), specificity(SPC),
positive and negative predictive values(PPV, NPV), accuracy(ACC)
and Matthews correlation coefficient (MCC).

| Marker | SEN | SPC | PPV | NPV | ACC | MCC |
|---|---|---|---|---|---|---|
| METloc001 | 0.93 | 0.86 | 0.85 | 0.93 | 0.89 | 0.7957 |
| METloc002 | 0.93 | 0.84 | 0.83 | 0.93 | 0.88 | 0.7733 |
| ARID3C | 0.96 | 0.76 | 0.76 | 0.96 | 0.85 | 0.7277 |
| ARL5C | 0.73 | 0.78 | 0.72 | 0.78 | 0.76 | 0.5619 |

TABLE 5
Classification statistics for combinations of METloc001, METloc002,
ARID3C and ARL5C for detecting the presence of vulvar, cervical,
endometrial, fallopian tube and ovarian cancer in whole tissue
sections. Sensitivity(SEN), specificity(SPC), positive and
negative predictive values(PPV, NPV), accuracy(ACC) and Matthews
correlation coefficient (MCC).

| Marker 1 | Marker 2 | SEN | SPC | PPV | NPV | ACC | MCC |
|---|---|---|---|---|---|---|---|
| METloc001 | METloc002 | 0.97 | 0.99 | 0.99 | 0.97 | 0.98 | 0.957 |
| METloc001 | ARID3C | 0.94 | 0.99 | 0.99 | 0.93 | 0.96 | 0.927 |
| METloc001 | ARL5C | 0.93 | 0.99 | 0.99 | 0.92 | 0.96 | 0.9193 |
| METloc002 | ARID3C | 0.94 | 0.98 | 0.98 | 0.93 | 0.96 | 0.9187 |
| METloc002 | ARL5C | 0.93 | 0.97 | 0.98 | 0.92 | 0.95 | 0.9027 |
| ARID3C | ARL5C | 0.95 | 0.9 | 0.93 | 0.92 | 0.93 | 0.8533 |

### Example 2. Detection of ARL5C and ARID3C Methylation in Cervical Normal, Precancer and Cancer Tissues

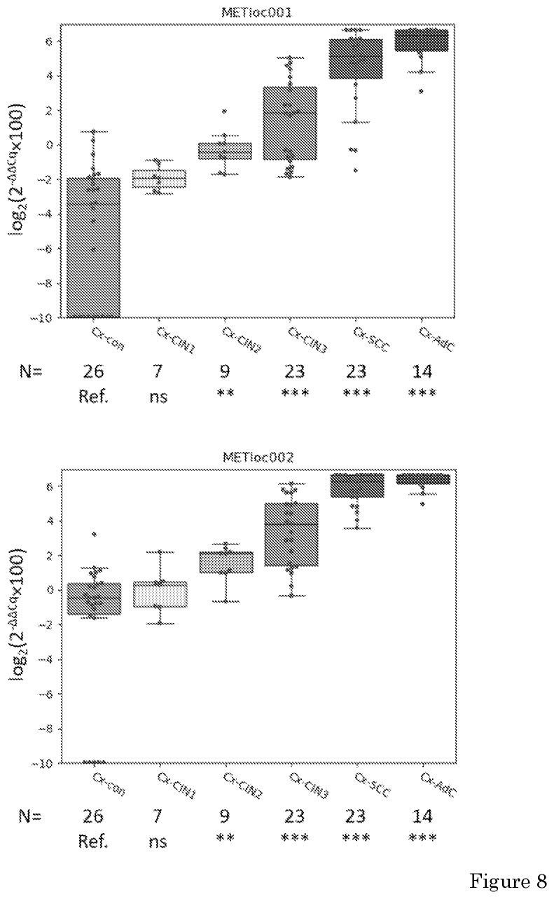

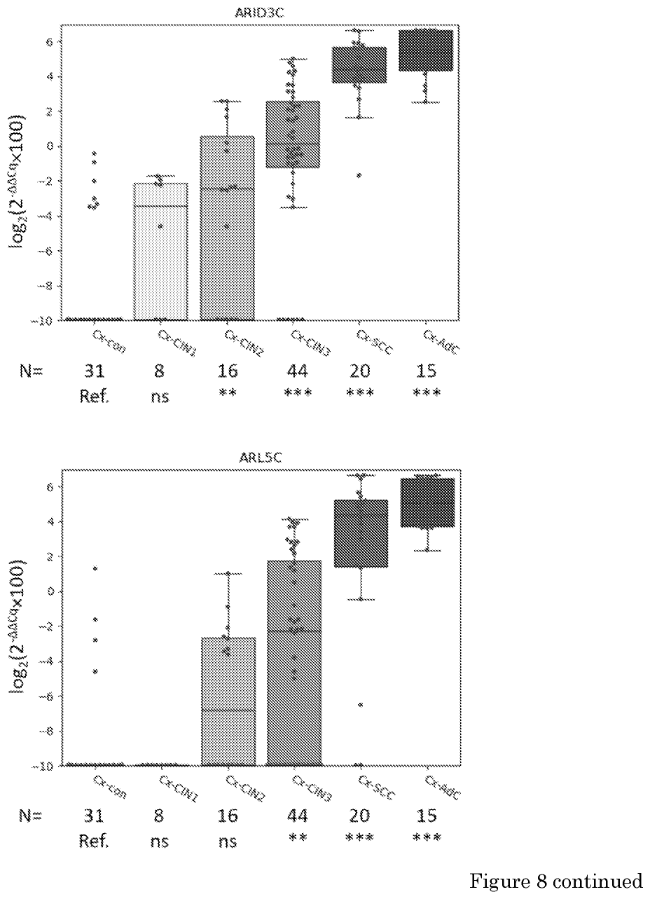

In this example we assessed the potential value of the DNA methylation of ARL5C and ARID3C as a marker for the detection of cervical cancer. For this purpose, the methylation levels of the markers ARL5C and ARID3C were analyzed in a test set of 134 cervical normal, precancer and cancer tissues by qMSP as described in MM. Of these 134 samples, 31 were classified as normal cervix (Cx-con), 8 as cervical intra-epithelial neoplasia 1 (Cx-CIN1), 16 as cervical intraepithelial neoplasia 2 (Cx-CIN2), 44 as cervical intra-epithelial neoplasia 3 (Cx-CIN3), 20 as cervical squamous cell carcinoma (Cx-SCC), and 15 as cervical adenocarcinoma (Cx-AdC). Methylation levels of both markers were calculated for each of the samples and the results are provided in FIG. 8. The methylation levels of both ARID3C and ARL5C significantly increased with increasing severity of the histological lesion.

### Example 2A. Detection of METloc001 and METloc002 Methylation in Cervical Normal, Precancer and Cancer Tissues

In this example we assessed the potential value of the DNA methylation of METloc001 and METloc002 as a marker for the detection of cervical cancer. For this purpose, the methylation levels of the markers METloc001 and METloc002 were analyzed in a test set of 102 cervical normal, precancer and cancer tissues by qMSP as described in MM. Of these 102 samples, 26 were classified as normal cervix (Cx-con), 7 as cervical intra-epithelial neoplasia 1 (Cx-CIN1), 9 as cervical intraepithelial neoplasia 2 (Cx-CIN2), 23 as cervical intra-epithelial neoplasia 3 (Cx-CIN3), 23 as cervical squamous cell carcinoma (Cx-SCC), and 14 as cervical adenocarcinoma (Cx-AdC). Methylation levels of both markers were calculated for each of the samples and the results are provided in FIG. 8. The methylation levels of both METloc001 and METloc002 significantly increased with increasing severity of the histological lesion.

### Example 3. Detection of ARL5C and ARID3C Methylation in Vulvar Normal, Precancer and Cancer Tissues

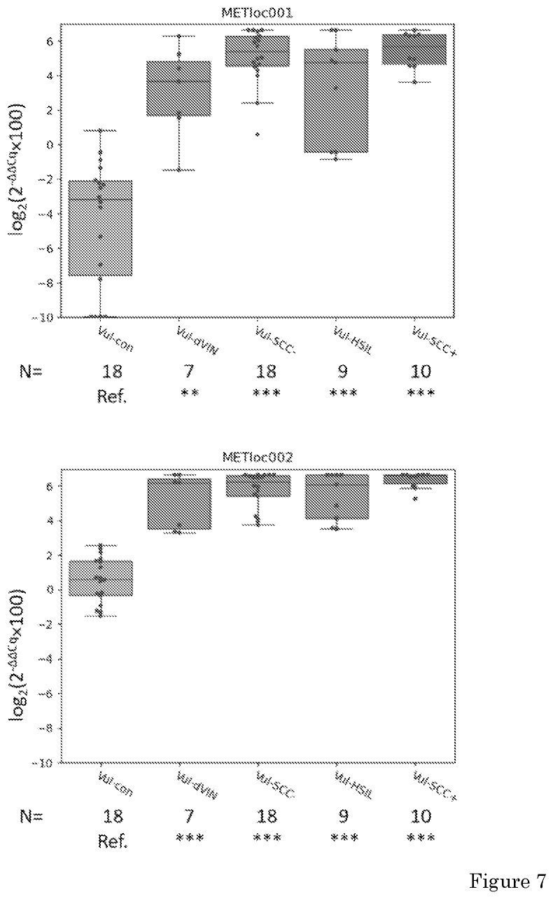

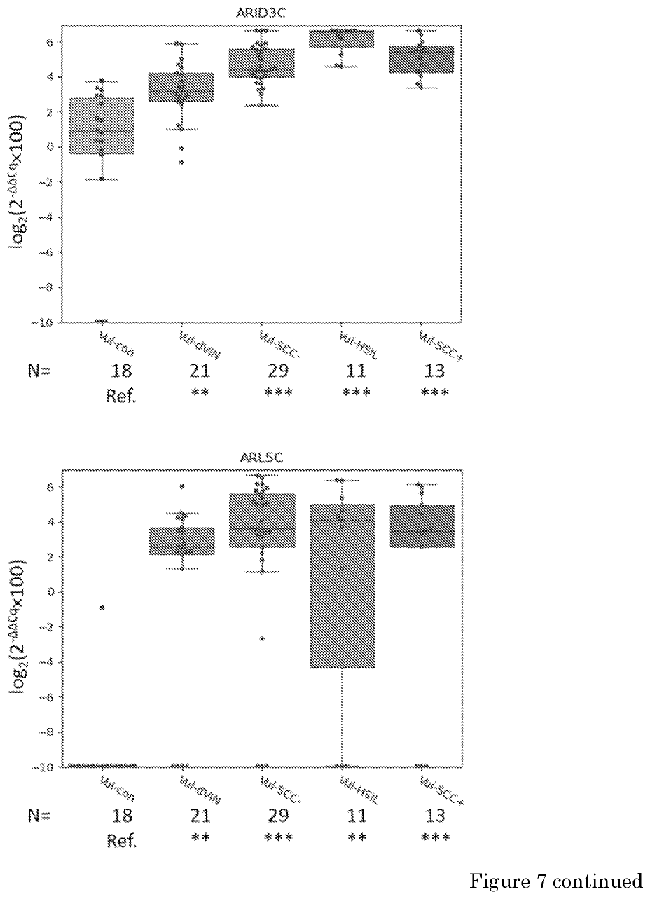

In this example we assessed the potential value of the DNA methylation of ARL5C and ARID3C as a marker for the detection of vulvar cancer. For this purpose, the methylation levels of ARL5C and ARID3C were analyzed in a test set of 92 vulvar normal, precancer and cancer tissues by qMSP as described in MM. Of these 92 samples, 18 were classified as normal vulva (Vul-con), 21 as differentiated vulvar intraepithelial neoplasia (Vul-dVIN), 11 as vulvar high-grade intraepithelial lesion (Vul-HSIL), 29 as HPV-negative squamous cell carcinoma (Vul-SSC−) and 13 as HPV-positive squamous cell carcinoma (Vul-SSC+). Methylation levels of both markers were calculated for each of the samples and the results are provided in FIG. 7. The methylation levels of both ARID3C and ARL5C were significantly increased if vulvar cancer or precancer was present.

### Example 3A. Detection of METloc001 and METloc002 Methylation in Vulvar Normal, Precancer and Cancer Tissues

In this example we assessed the potential value of the DNA methylation of METloc001 and METloc002 as a marker for the detection of vulvar cancer. For this purpose, the methylation levels of METloc001 and METloc002 were analyzed in a test set of 62 vulvar normal, precancer and cancer tissues by qMSP as described in MM. Of these 62 samples, 18 were classified as normal vulva (Vul-con), 7 as differentiated vulvar intraepithelial neoplasia (Vul-dVIN), 9 as vulvar high-grade intraepithelial lesion (Vul-HSIL), 18 as HPV-negative squamous cell carcinoma (Vul-SSC−) and 10 as HPV-positive squamous cell carcinoma (Vul-SSC+). Methylation levels of both markers were calculated for each of the samples and the results are provided in FIG. 7. The methylation levels of both METloc001 and METloc002 were significantly increased if vulvar cancer or precancer was present.

### Example 4. Detection of ARL5C and ARID3C Methylation in Endometrial Normal and Cancer Tissues

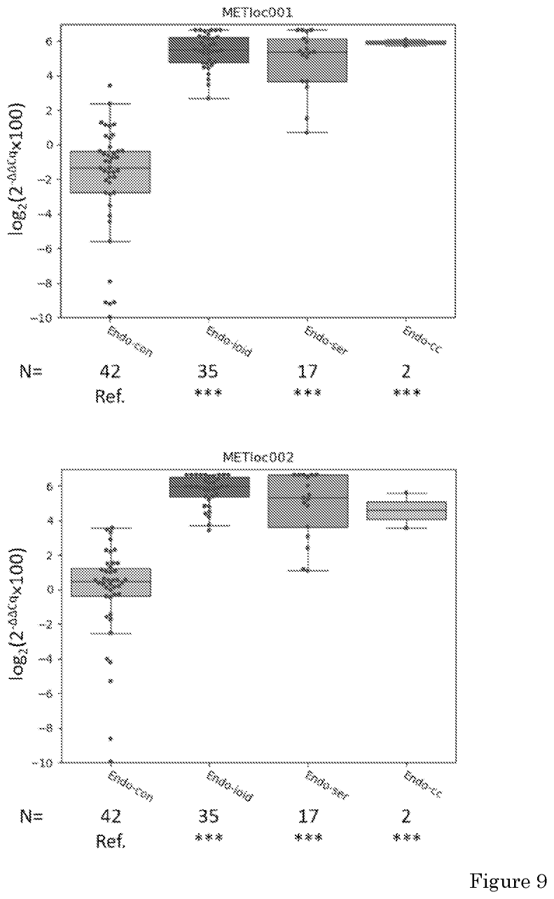

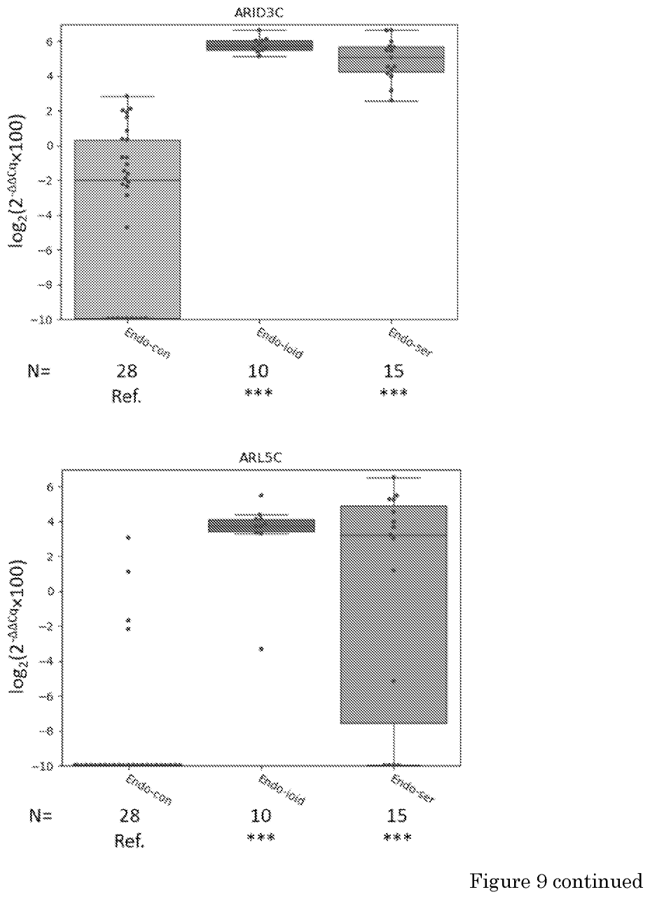

In this example we assessed the potential value of the DNA methylation of ARL5C and ARID3C as a marker for the detection of endometrial cancer. For this purpose, the methylation levels of ARL5C and ARID3C were analyzed in a test set of 53 endometrial normal and cancer tissues by qMSP as described in MM. Of these 53 samples, 28 were classified as normal endometrium (Endo-con), 10 as endometrial endometrioid (Endo-ioid), 15 as endometrial serous (Endo-ser.). Methylation levels of both markers were calculated for each of the samples and the results are provided in FIG. 9. The methylation levels of both ARID3C and ARL5C were significantly increased if endometrial cancer was present.

### Example 4A. Detection of METloc001 and METloc002 Methylation in Endometrial Normal and Cancer Tissues

In this example we assessed the potential value of the DNA methylation of METloc001 and METloc002 as a marker for the detection of endometrial cancer. For this purpose, the methylation levels of METloc001 and METloc002 were analyzed in a test set of 96 endometrial normal and cancer tissues by qMSP as described in MM. Of these 96 samples, 42 were classified as normal endometrium (Endo-con), 35 as endometrial endometrioid (Endo-ioid) and 17 as endometrial serous (Endo-ser.) and 2 as endometrial clear cell carcinoma (Endo-cc). Methylation levels of both markers were calculated for each of the samples and the results are provided in FIG. 9. The methylation levels of both METloc001 and METloc002 were significantly increased if endometrial cancer was present.

### Example 5. Detection of ARL5C and ARID3C Methylation in Ovarian Normal and Cancer Tissues

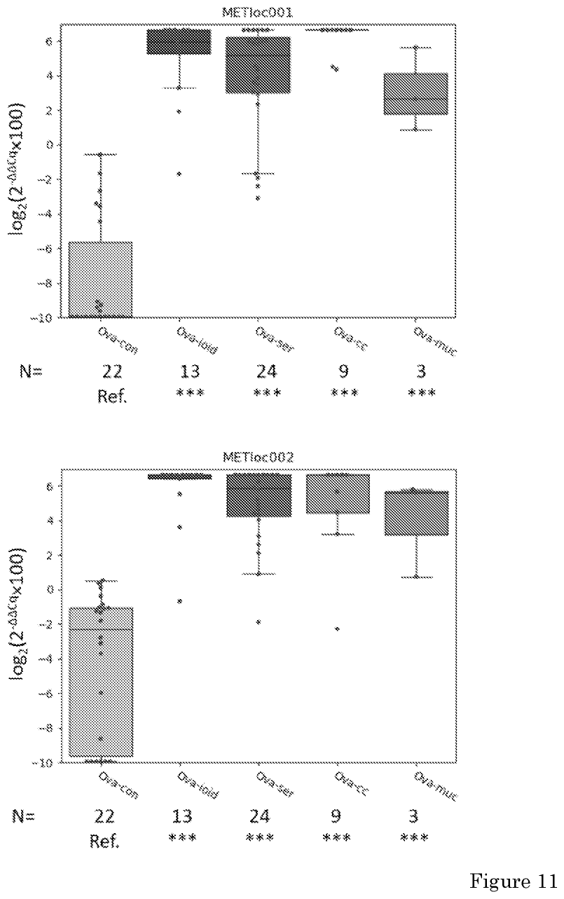

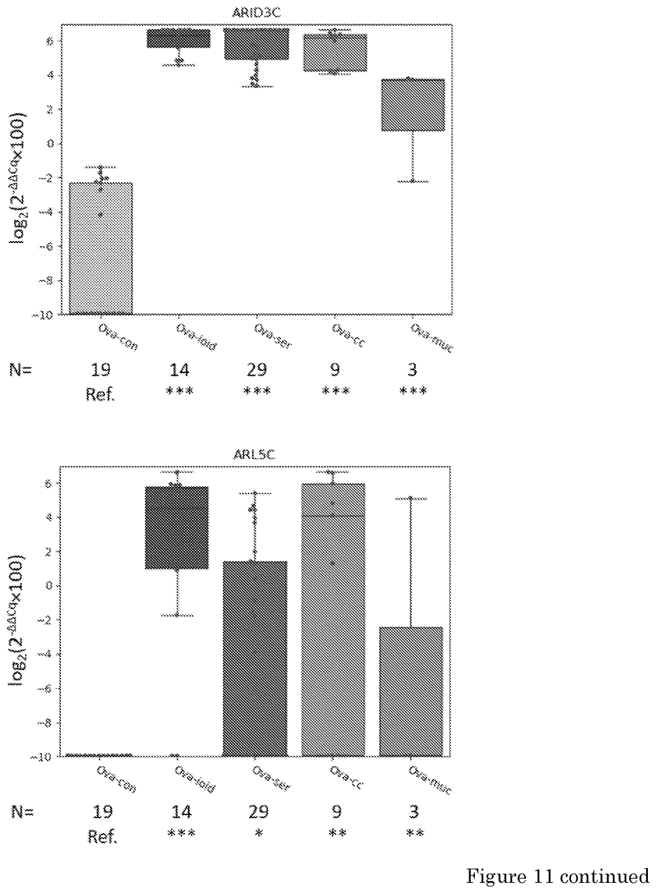

In this example we assessed the potential value of the DNA methylation of ARL5C and ARID3C as a marker for the detection of ovarian cancer. For this purpose, the methylation levels of ARL5C and ARID3C in a test set of 74 ovarian normal and cancer tissues were analyzed by qMSP as described in MM. Of these 74 samples, 19 were classified as normal ovarium (Ova-con), 14 as ovarian endometrioid (Ova-ioid), 29 as ovarian high-grade serous (Ova-ser), 9 as ovarian clear-cell (Ova-cc) and 3 as ovarian mucinous (Ova-muc). Methylation levels of both markers were calculated for each of the samples and the results are provided in FIG. 11. The methylation levels of both ARID3C and ARL5C were significantly increased if ovarian cancer was present.

### Example 5A Detection of METloc001 and METloc002 Methylation in Ovarian Normal and Cancer Tissues

In this example we assessed the potential value of the DNA methylation of METloc001 and METloc002 as a marker for the detection of ovarian cancer. For this purpose, the methylation levels of METloc001 and METloc002 in a test set of 71 ovarian normal and cancer tissues were analyzed by qMSP as described in MM. Of these 71 samples, 22 were classified as normal ovarium (Ova-con), 13 as ovarian endometrioid (Ova-ioid), 24 as ovarian high-grade serous (Ova-ser), 9 as ovarian clear-cell (Ova-cc) and 3 as ovarian mucinous (Ova-muc). Methylation levels of both markers were calculated for each of the samples and the results are provided in FIG. 11. The methylation levels of both METloc001 and METloc002 were clearly increased if ovarian cancer was present.

### Example 6. Detection of ARL5C and ARID3C Methylation in Fallopian Tube Normal and Cancer Tissues

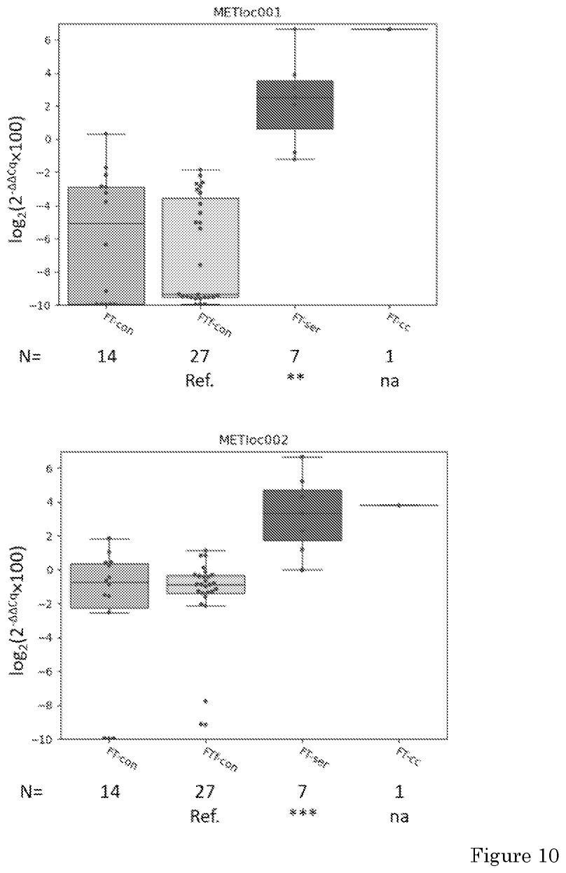

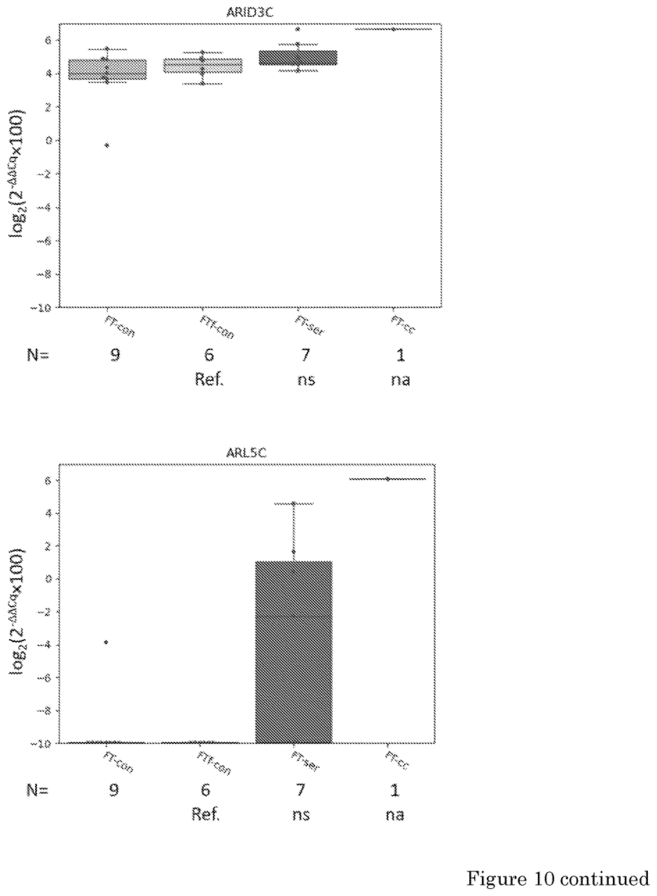

In this example we assessed the potential value of the DNA methylation of ARL5C and ARID3C as a marker for the detection of fallopian tube cancer. For this purpose, the methylation levels of ARL5C and ARID3C in a test set of 23 fallopian tube normal and cancer tissues were analyzed by qMSP as described in MM. Of these 23 samples, 9 were classified as normal fallopian tube (FT-con), 6 as normal fallopian tube and fimbriae (FTf-con), 7 as fallopian tube serous (FT-ser), and 1 as fallopian tube clear-cell (FT-cc). Methylation levels of both markers were calculated for each of the samples and the results are provided in FIG. 10. The methylation levels of both ARID3C and ARL5C were clearly increased if fallopian tube cancer was present.

### Example 6A Detection of METloc001 and METloc002 Methylation in Fallopian Tube Normal and Cancer Tissues

In this example we assessed the potential value of the DNA methylation of METloc001 and METloc002 as a marker for the detection of fallopian tube cancer. For this purpose, the methylation levels of METloc001 and METloc002 in a test set of 49 fallopian tube normal and cancer tissues were analyzed by qMSP as described in MM. Of these 49 samples, 14 were classified as normal fallopian tube (FT-con), 27 as normal fallopian tube and fimbriae (FTf-con), 7 as fallopian tube serous (FT-ser), and 1 as fallopian tube clear-cell (FT-cc). Methylation levels of both markers were calculated for each of the samples and the results are provided in FIG. 10. The methylation levels of both METloc001 and METloc002 were significantly increased if fallopian tube cancer was present.

### Example 7. Detection of ARL5C and ARID3C Methylation in Head and Neck Normal and Cancer Tissues

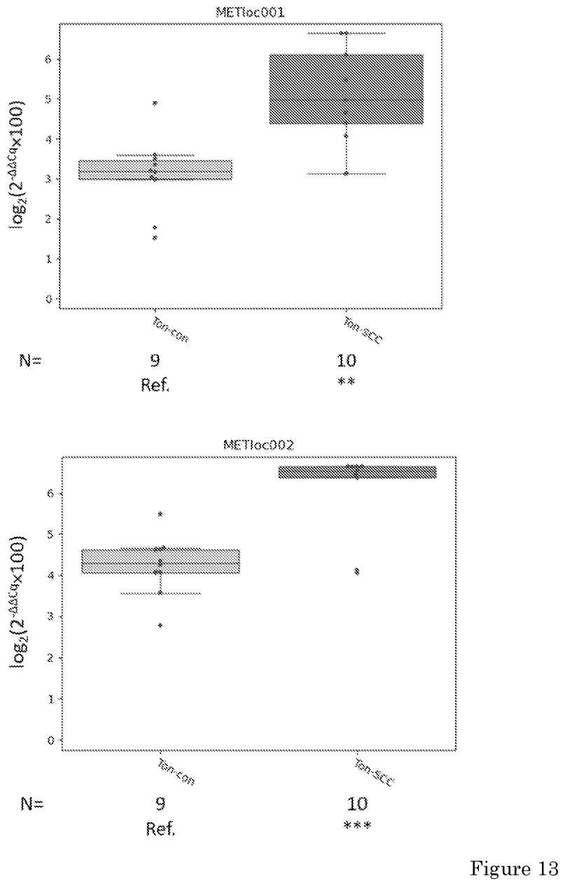

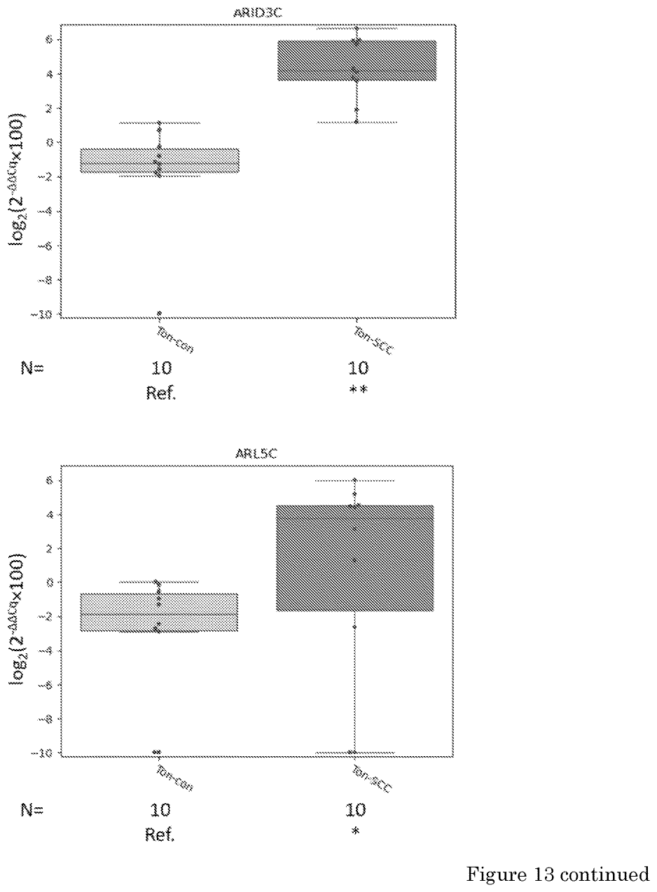

In this example we assessed the potential value of the DNA methylation of ARL5C and ARID3C as a marker for the detection of head and neck cancer. For this purpose, the methylation levels of ARL5C and ARID3C in a test set of 20 tonsillar formalin fixed paraffin embedded (FFPE) whole tissue samples (WTS) were analyzed by qMSP as described in MM. Of these 20 samples, 10 were classified as normal tonsil (Ton-con) and 10 as tonsil squamous cell carcinoma (Ton-SCC). Methylation levels of both markers were calculated for each of the samples and the results are provided in FIG. 13. The methylation levels of both ARID3C and ARL5C were significantly increased if cancer was present.

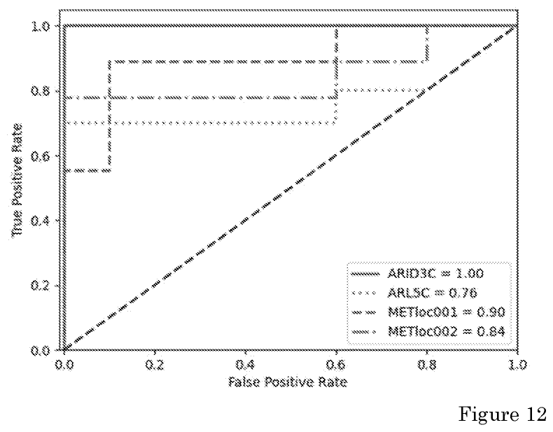

ROC curve analysis and area under the curve (AUC) of methylation ratios for detecting tonsil cancer is shown in FIG. 12. Table 6 shows the sensitivity, specificity, positive and negative predictive values, accuracy and Matthews correlation coefficient.

TABLE 6
Classification statistics for detecting tonsil cancer in
whole tissue sections. Sensitivity(SEN), specificity(SPC),
positive and negative predictive values(PPV, NPV), accuracy(ACC)
and Matthews correlation coefficient (MCC).

| Marker | SEN | SPC | PPV | NPV | ACC | MCC |
|---|---|---|---|---|---|---|
| METloc001 | 0.89 | 0.9 | 0.89 | 0.9 | 0.89 | 0.7778 |
| METloc002 | 0.78 | 1 | 1 | 0.83 | 0.89 | 0.7821 |
| ARID3C | 1 | 1 | 1 | 1 | 1 | 1 |
| ARL5C | 0.7 | 1 | 1 | 0.77 | 0.85 | 0.7024 |

### Example 7A. Detection of METloc001 and METloc002 Methylation in Head and Neck Normal and Cancer Tissues

In this example we assessed the potential value of the DNA methylation of METloc001 and METloc002 as a marker for the detection of head and neck cancer. For this purpose, the methylation levels of METloc001 and METloc002 in a test set of 19 tonsillar formalin fixed paraffin embedded (FFPE) whole tissue samples (WTS) were analyzed by qMSP as described in MM. Of these 19 samples, 9 were classified as normal tonsil (HN con) and 10 as tonsil squamous cell carcinoma (HN car). Methylation levels of both markers were calculated for each of the samples and the results are provided in FIG. 13. The methylation levels of both METloc001 and METloc002 were significantly increased if cancer was present.

ROC curve analysis and area under the curve (AUC) of methylation ratios for detecting tonsil cancer is shown in FIG. 12. Table 6 shows the sensitivity, specificity, positive and negative predictive values and Matthews correlation coefficient.

### Example 8. Detection of ARL5C and ARID3C Methylation in Colorectal and Anal Normal and Cancer Tissues

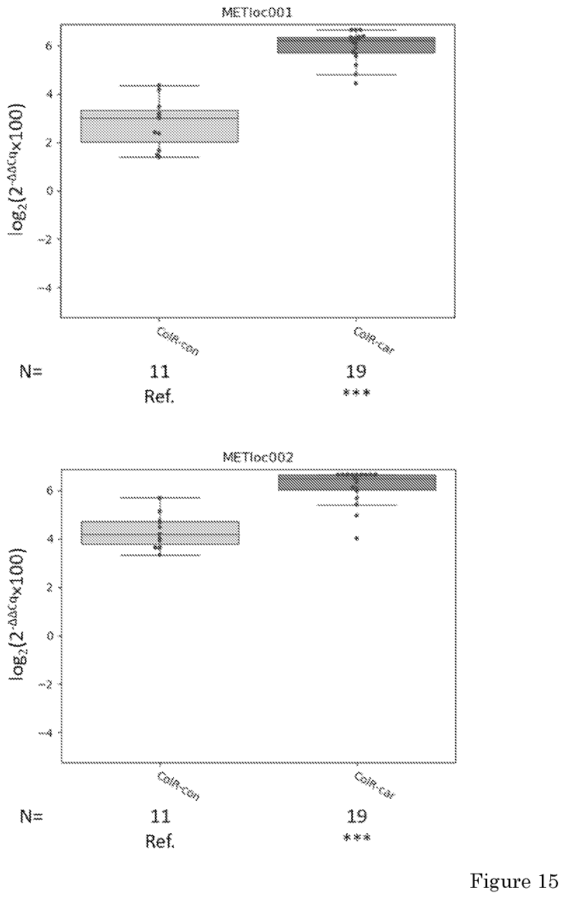

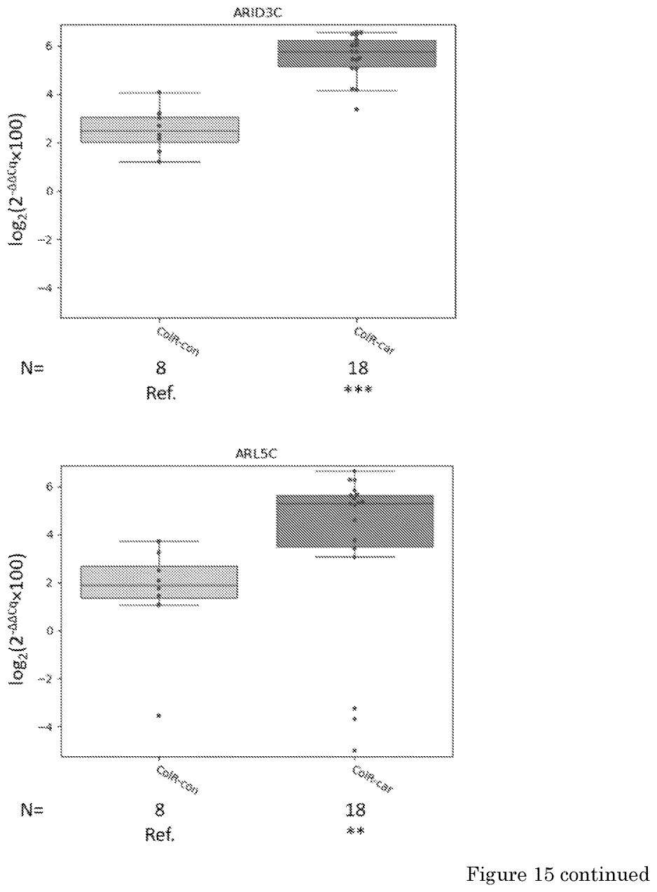

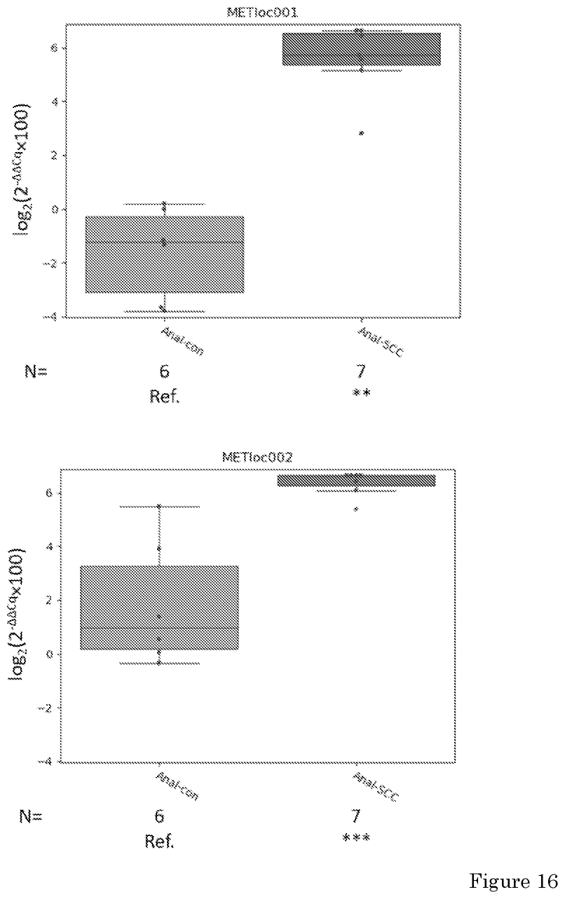

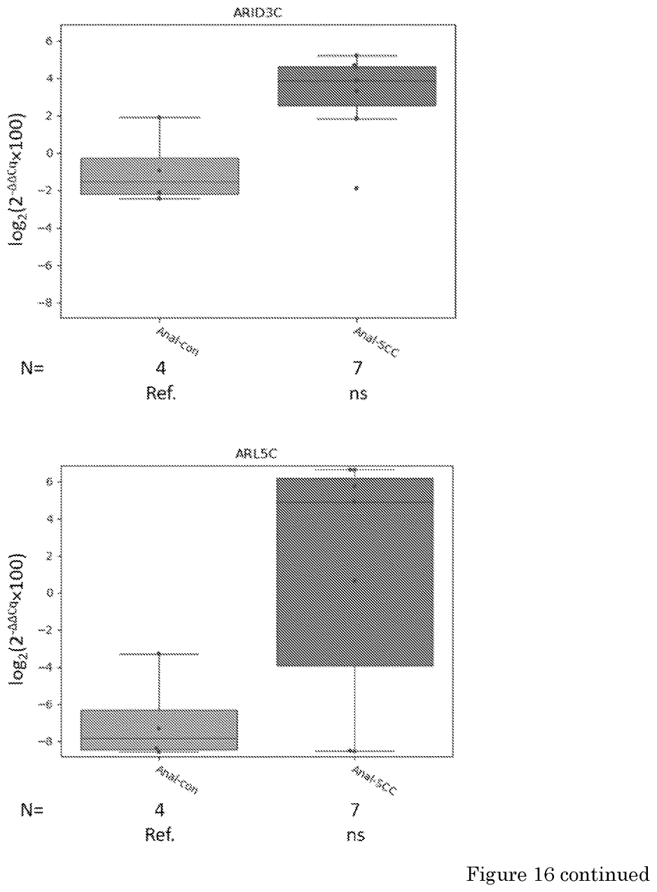

In this example we assessed the potential value of the DNA methylation of ARL5C and ARID3C as a marker for the detection of colorectal cancer. For this purpose, the methylation levels of ARL5C and ARID3C in a test set of 26 colorectal formalin fixed paraffin embedded (FFPE) whole tissue samples (WTS) were analyzed by qMSP as described in MM. Of these 26 samples, 8 were classified as normal colorectal tissue (ColR-con) and 18 as colorectal carcinoma (ColR-car). Further, the methylation levels of ARL5C and ARID3C were analyzed by qMSP in 11 anal formalin fixed paraffin embedded (FFPE) whole tissue samples (WTS). Of these 11 sample, 4 were classified as normal anal (Anal-con) and 7 were classified as anal squamous cell carcinoma (Anal-SCC). Methylation levels of both markers were calculated for each of the samples and the results are provided in FIGS. 15 and 16. The methylation levels of both ARID3C and ARL5C were (significantly) increased if colorectal or anal cancer was present.

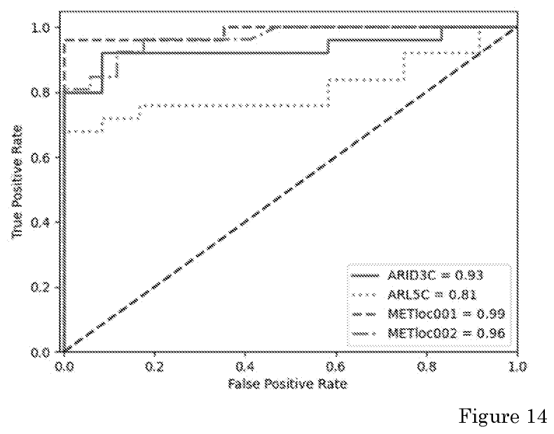

ROC curve analysis and area under the curve (AUC) of methylation ratios for detecting colorectal and anal cancer is shown in FIG. 14. Table 7 shows the sensitivity, specificity, positive and negative predictive values, accuracy and Matthews correlation coefficient.

TABLE 7
Classification statistics for detecting colorectal and anal
cancers in whole tissue sections. Sensitivity(SEN), specificity(SPC),
positive and negative predictive values(PPV, NPV), accuracy(ACC)
and Matthews correlation coefficient (MCC).

| Marker | SEN | SPC | PPV | NPV | ACC | MCC |
|---|---|---|---|---|---|---|
| METloc001 | 0.96 | 1 | 1 | 0.94 | 0.98 | 0.9507 |
| METloc002 | 0.69 | 1 | 1 | 0.68 | 0.81 | 0.6682 |
| ARID3C | 0.92 | 0.92 | 0.96 | 0.85 | 0.92 | 0.8172 |
| ARL5C | 0.68 | 1 | 1 | 0.6 | 0.78 | 0.6137 |

### Example 8A. Detection of METloc001 and METloc002 Methylation in Colorectal and Anal Normal and Cancer Tissues

In this example we assessed the potential value of the DNA methylation of METloc001 and METloc002 as a marker for the detection of colorectal cancer. For this purpose, the methylation levels of METloc001 and METloc002 in a test set of 30 colorectal formalin fixed paraffin embedded (FFPE) whole tissue samples (WTS) were analyzed by qMSP as described in MM. Of these 30 samples, 11 were classified as normal colorectal tissue (ColR-con) and 19 as colorectal carcinoma (ColR-car). Further, the methylation levels of METloc001 and METloc002 were analyzed by qMSP in 13 anal formalin fixed paraffin embedded (FFPE) whole tissue samples (WTS). Of these 13 sample, 6 were classified as normal anal (Anal-con) and 7 were classified as anal squamous cell carcinoma (Anal-SCC). Methylation levels of both markers were calculated for each of the samples and the results are provided in FIGS. 15 and 16. The methylation levels of both METloc001 and METloc002 were significantly increased if colorectal or anal cancer was present. ROC curve analysis and area under the curve (AUC) of methylation ratios for detecting tonsil cancer is shown in FIG. 14. Table 7 shows the sensitivity, specificity, positive and negative predictive values, accuracy and Matthews correlation coefficient.

### Example 9. Detection of METloc001 and METloc002 Methylation in Skin Cancer

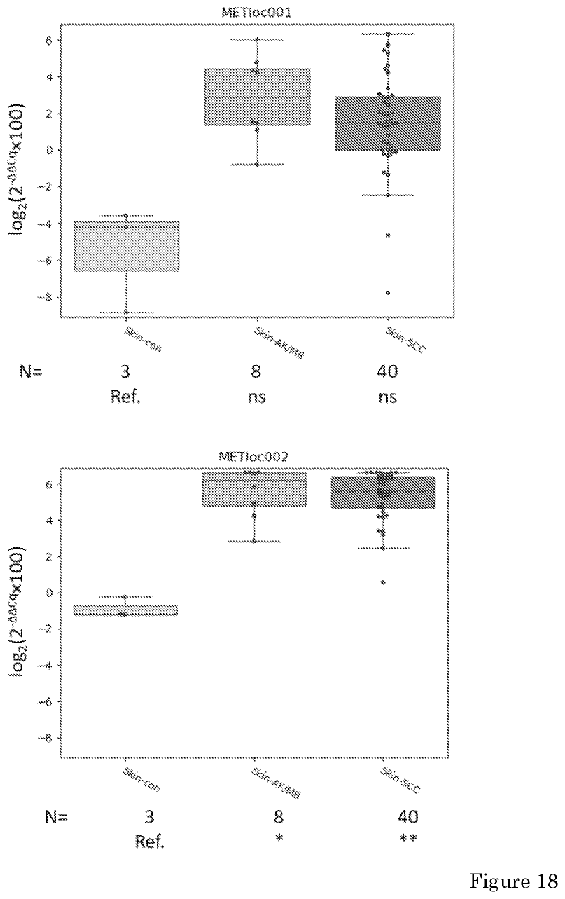

In this example we assessed the potential value of the DNA methylation of METloc001 and METloc002 as a marker for the detection of skin cancer. For this purpose, the methylation levels of METloc001 and METloc002 in a test set of 51 cutaneous formalin fixed paraffin embedded (FFPE) whole tissue samples (WTS) were analyzed by qMSP as described in MM. Of these 51 samples, 3 were classified as normal skin (Skin-con), 8 as precancer (Actinic Keratosis (AK), Bowen's disease (MB; Morbus Bowen)) and 40 as skin squamous cell carcinoma (Skin-SCC). Methylation levels of both markers were calculated for each of the samples and the results are provided in FIG. 18. The methylation levels of both METloc001 and METloc002 were clearly increased if skin cancer was present.

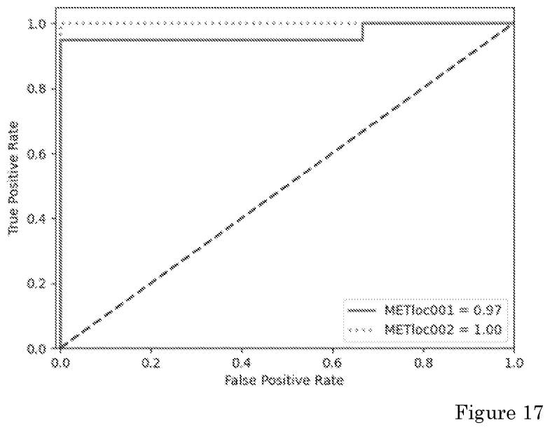

ROC curve analysis and area under the curve (AUC) of methylation ratios for detecting skin cancer is shown in FIG. 17. Table 8 shows the sensitivity, specificity, positive and negative predictive values, accuracy and Matthews correlation coefficient.

TABLE 8
Classification statistics for METloc001 and METloc002 for detecting
skin cancer in whole tissue sections. Sensitivity(SEN), specificity(SPC),
positive and negative predictive values(PPV, NPV), accuracy(ACC)
and Matthews correlation coefficient (MCC).

| Marker | SEN | SPC | PPV | NPV | ACC | MCC |
|---|---|---|---|---|---|---|
| METloc001 | 0.95 | 0.27 | 0.83 | 0.6 | 0.8 | 0.3269 |
| METloc002 | 1 | 0.27 | 0.83 | 1 | 0.84 | 0.4449 |

### Example 10. Detection of ARL5C and ARID3C Methylation in Physician Taken Cytological Samples

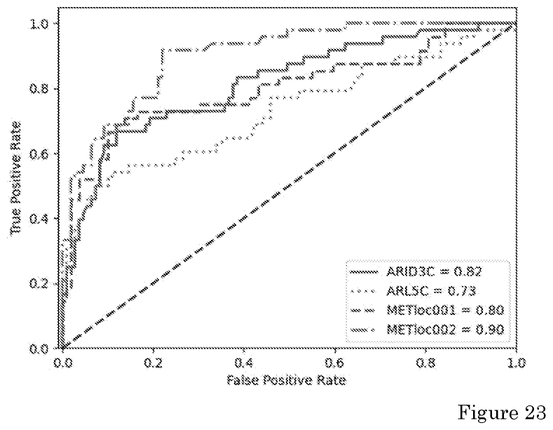

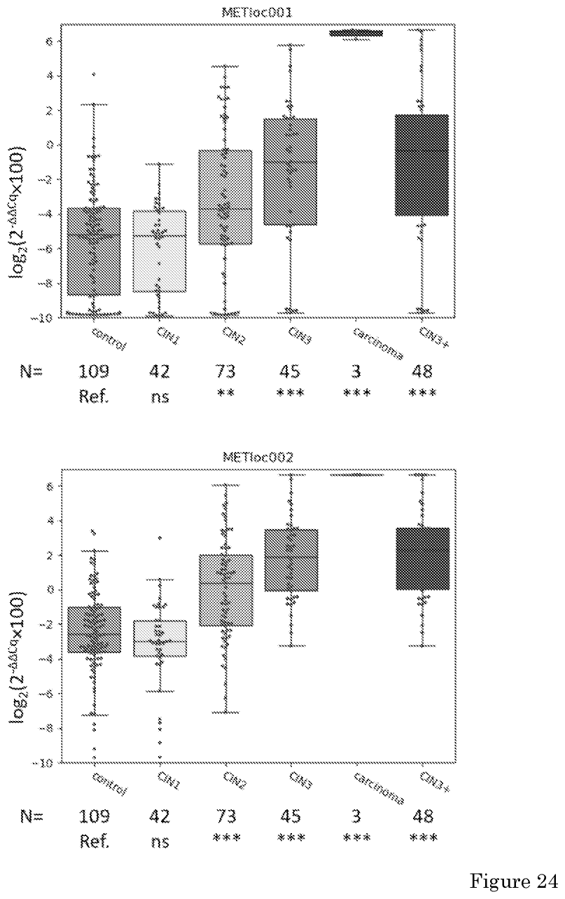

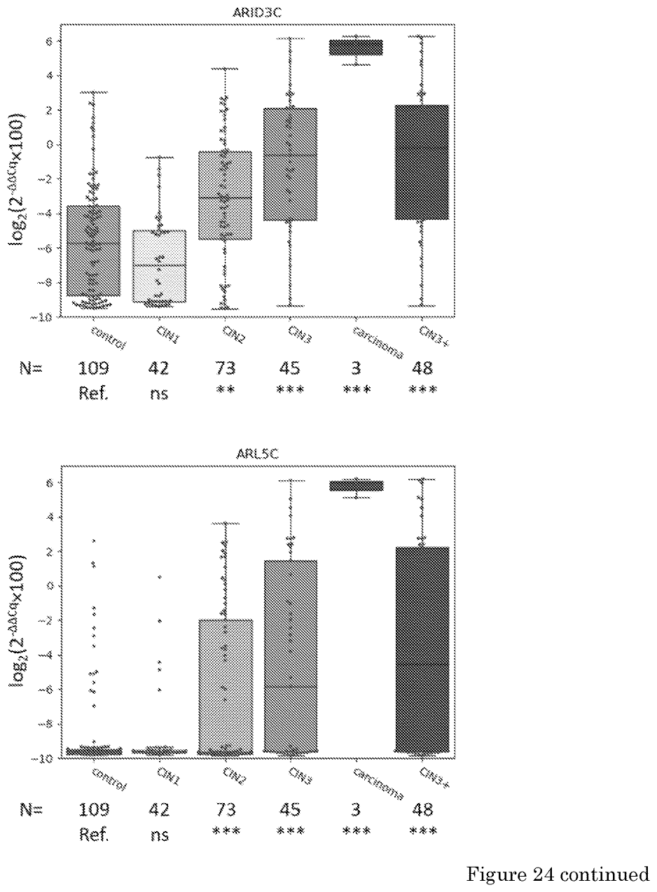

In this example we assessed the potential value of the DNA methylation of ARL5C and ARID3C as a marker for the detection of cervical cancer in physician taken cytological samples. For this purpose, the methylation levels of ARL5C and ARID3C in a test set of 272 physician taken cytological samples were analyzed by qMSP as described in MM. These samples are part of the EVAH-study, a follow-up study of woman referred to the colposcopy clinic of Hospital Clinic, Barcelona, Spain because of abnormal cytology (≥ASC-US). This study is registered in the Dutch Trial Register (NTR3464). Of these 272 samples, 109 were classified as CIN 0, 42 as CIN1, 73 as CIN2, 45 as CIN 3, and 3 as carcinoma. Methylation levels of both markers were calculated for each of the samples and the results are provided in FIGS. 23 and 24. The methylation levels of both ARID3C and ARL5C significantly increased with increasing severity of the histological lesion.

### Example 10A. Detection of METloc001 and METloc002 Methylation in Physician Taken Cytological Samples

In this example we assessed the potential value of the DNA methylation of METloc001 and METloc002 as a marker for the detection of cervical cancer in physician taken cytological samples. For this purpose, the methylation levels of METloc001 and METloc002 in a test set of 272 physician taken cytological samples were analyzed by qMSP as described in MM. These samples are part of the EVAH-study, a follow-up study of woman referred to the colposcopy clinic of Hospital Clinic, Barcelona, Spain because of abnormal cytology (≥ASC-US). This study is registered in the Dutch Trial Register (NTR3464). Of these 272 samples, 109 were classified as CIN 0, 42 as CIN1, 73 as CIN2, 45 as CIN 3, and 3 as carcinoma. Methylation levels of both markers were calculated for each of the samples and the results are provided in FIGS. 23 and 24. The methylation levels of both METloc001 and METloc002 significantly increased with increasing severity of the histological lesion.

Tables 11 and 12 show the sensitivity, specificity, positive and negative predictive values, accuracy and Matthews correlation coefficient individual (table 11) and combinations (table 12) of markers METloc001, METloc002, ARID3C and ARL5C for detecting CIN3+ in physician taken cytology samples. Table 13 shows correlation coefficient between METloc001, METloc002, ARID3C and ARL5C qMSP results of physician-taken cytology.

TABLE 11
Classification statistics for METloc001, METloc002, ARID3C
and ARL5C for detecting CIN3+ in physician taken
cytology samples. Sensitivity(SEN), specificity(SPC),
positive and negative predictive values(PPV, NPV), accuracy(ACC)
and Matthews correlation coefficient (MCC).

| Marker | SEN | SPC | PPV | NPV | ACC | MCC |
|---|---|---|---|---|---|---|
| METloc001 | 0.71 | 0.8 | 0.44 | 0.93 | 0.79 | 0.4753 |
| METloc002 | 0.92 | 0.67 | 0.37 | 0.97 | 0.71 | 0.4666 |
| ARID3C | 0.67 | 0.79 | 0.41 | 0.92 | 0.77 | 0.4417 |
| ARL5C | 0.44 | 0.88 | 0.43 | 0.88 | 0.8 | 0.3747 |

TABLE 12
Classification statistics for combinations of METloc001,
METloc002, ARID3C and ARL5C for detecting CIN3+ in
physician taken cytology samples. Sensitivity(SEN), specificity(SPC),
positive and negative predictive values(PPV, NPV), accuracy(ACC)
and Matthews correlation coefficient (MCC).

| Marker 1 | Marker 2 | SEN | SPC | PPV | NPV | ACC | MCC |
|---|---|---|---|---|---|---|---|
| METloc001 | METloc002 | 0.94 | 0.76 | 0.63 | 0.97 | 0.82 | 0.6557 |
| METloc001 | ARID3C | 0.79 | 0.83 | 0.67 | 0.9 | 0.82 | 0.6209 |
| METloc001 | ARL5C | 0.79 | 0.82 | 0.66 | 0.9 | 0.81 | 0.6116 |
| METloc002 | ARID3C | 0.92 | 0.78 | 0.65 | 0.96 | 0.82 | 0.6597 |
| METloc002 | ARL5C | 0.94 | 0.77 | 0.64 | 0.97 | 0.82 | 0.6647 |
| ARID3C | ARL5C | 0.71 | 0.88 | 0.72 | 0.87 | 0.83 | 0.6224 |

TABLE 13
Pearson correlation coefficient between METloc001,
METloc002, ARID3C and ARL5C qMSP results of
physician-taken cytology results.

| | METloc001 | METloc002 | ARID3C | ARL5C |
|---|---|---|---|---|
| METloc001 | 1.00 | 0.91 | 0.88 | 0.89 |
| METloc002 | 0.91 | 1.00 | 0.90 | 0.87 |
| ARID3C | 0.88 | 0.90 | 1.00 | 0.96 |
| ARL5C | 0.89 | 0.87 | 0.96 | 1.00 |

### Example 11. METloc009 (Cervical and Vulvar Cancer), GLIS3 (Vulvar Cancer) and FRMD4B (Cervical Adenocarcinoma) as Methylation Markers for Regional or Specific Cancers

H&E stainings of tumor and normal tissue sections were reviewed by a pathologist to confirm correct classification and to annotate tumor and corresponding normal tissue regions on the sections for LCM. The annotated regions were then laser capture microdissected for target cell enrichment. The genomic DNA from the microdissected tissues was extracted by proteinase K, and the extracted DNA was used for MeD-seq analysis.

By comparing methylation profiles of vulvar, cervical, endometrial, fallopian tube and ovarian carcinoma and the methylation profiles of corresponding normal tissue (for example see FIG. 1), we identified DMR's that displayed higher levels of methylation in regional or specific cancers compared to the levels of methylation in the other cancers and normal tissues: METloc009 (cervical and vulvar cancer), GLIS3 (vulvar cancer) and FRMD4B (cervical adenocarcinoma). The DMR's in the regions indicated in table 1A are promising to be used for epigenetic marker development.

The usability of the methylation markers for distinguishing (pre)cancerous tissue from normal tissue was investigated by testing a series of biopsies, including vulvar, cervical, endometrial, fallopian tube and ovarian carcinoma and corresponding normal tissue, in qMSP assays. Primers and probes for these qMSP assays are shown in Table 15.

TABLE 15
Primer and probe sequences (5′-3′) used for qMSP analysis. Probe
with MGB incorporate a 5′ fluorescent dye and a minor groove binder
(MGB) conjugated to the 3′ non-fluorescent quencher of the probe.

| Oligo | Sequence |
|---|---|
| METloc009 Forward primer | TTATAGAATTCGCGGCGTAGC |
| METloc009 Reverse primer | TCAATTCACAACCGACCG |
| METloc009 Probe with MGB | CAAACGACCGCTATT |
| GLIS3 Forward primer | AGAGGGAGATATAAGTTCGGTTTTC |
| GLIS3 Reverse primer | AACTACTTCCTAAATATAAACGCCGAA |
| GLIS3 Probe with MGB | CGCGAGTTCGTTTTT |
| FRMD4B Forward primer | GGAGAGAGGGAGTCGAAAGTTTC |
| FRMD4B Reverse primer | TTACAACTATACCCCGAACCG |
| FRMD4B Probe with MGB | TCCTTCCGTCCTACCTA |
| ACTB Forward primer | TGGTGATGGAGGAGGTTTAGTAAGT |
| ACTB Reverse primer | AACCAATAAAACCTACTCCTCCCTTAA |
| ACTB Probe | ACCACCACCCAACACACAATAACAAACACA |

The ROC curve analysis of the qMSP was designed on basis of log 2-transformed MeD-seq read counts determined in cancerous tissues (n=116) and corresponding normal tissues (n=67) (See FIG. 4).

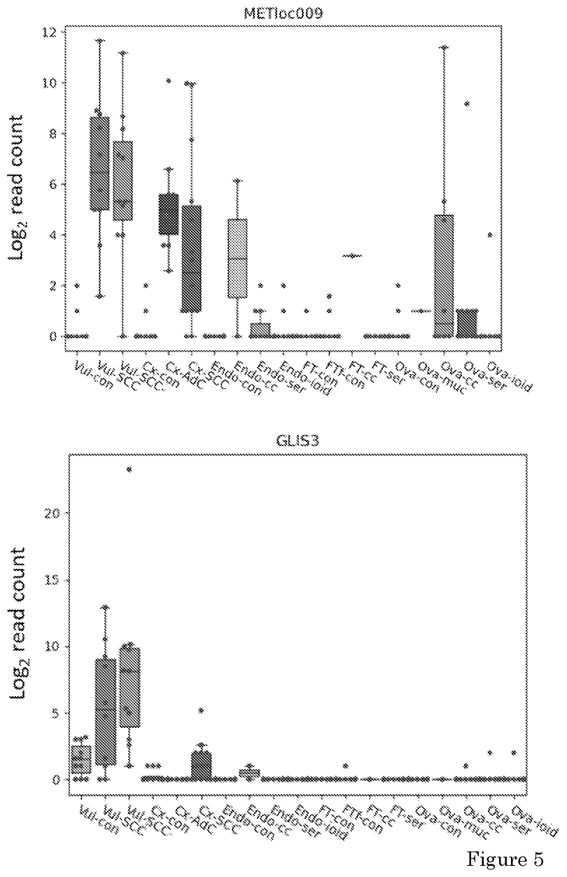

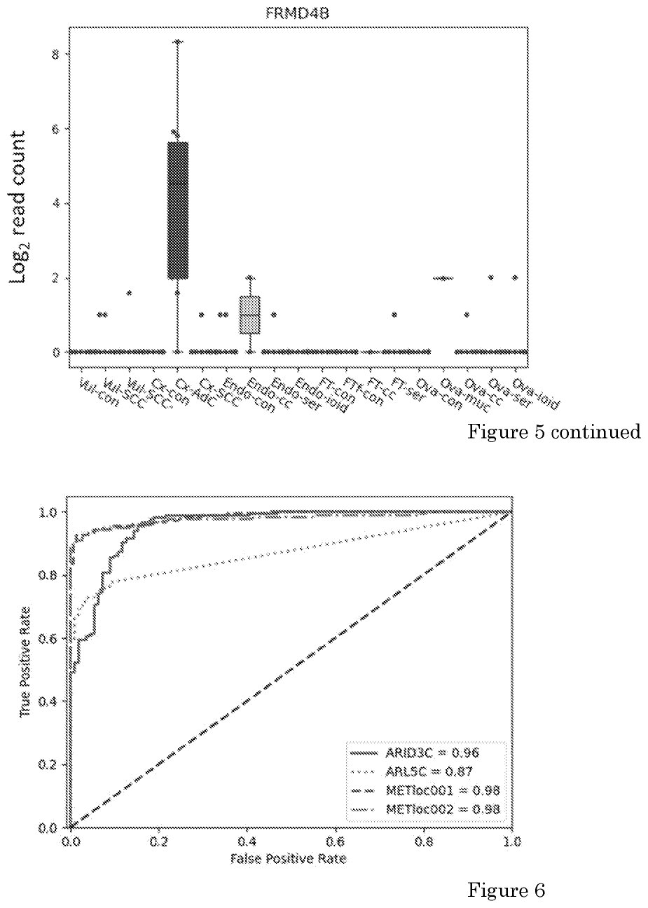

FIG. 5 shows the comparison of the MeD-seq read counts for METloc009; cervix and vulvar cancer, GLIS3; vulvar cancer and FRMD4B; cervical adenocarcinoma in LCM samples.

Table 3 shows the sensitivity, specificity, positive and negative predictive values, accuracy and Matthews correlation coefficient of MeD-seq read counts for METloc009; cervical and vulvar cancer, GLIS3; vulvar cancer and FRMD4B; cervical adenocarcinoma.

TABLE 3
Classification statistics of MeD-seq read counts for METloc009;
cervical and vulvar cancer, GLIS3; vulvar cancer and FRMD4B;
cervical adenocarcinoma. Sensitivity(SEN), specificity(SPC),
positive and negative predictive values(PPV, NPV), accuracy(ACC)
and Matthews correlation coefficient (MCC).

| Marker | SEN | SPC | PPV | NPV | ACC | MCC |
|---|---|---|---|---|---|---|
| METloc009 | 0.81 | 0.95 | 0.84 | 0.93 | 0.91 | 0.7755 |
| GLIS3 | 0.81 | 0.92 | 0.57 | 0.97 | 0.91 | 0.6367 |
| FRMD4B | 0.92 | 0.97 | 0.69 | 0.99 | 0.97 | 0.7773 |

METloc009 methylation was determined in cervical cancer and vulvar cancer. GLIS3 methylation was determined in vulvar cancer. FRMD4B methylation was determined in cervical cancer. For this purpose, the methylation levels were analyzed by qMSP as described in MM in test sets of:
54 cervical normal and cancer tissues; of these 54 samples, 20 were classified as normal cervix (Cx-con), 20 as cervical squamous cell carcinoma (Cx-SCC), and 14 as cervical adenocarcinoma (Cx-AdC)
37 vulvar normal, and cancer tissues; of these 37 samples, 18 were classified as normal vulva (Vul-con), 10 as HPV-negative squamous cell carcinoma (Vul-SSC−) and 9 as HPV-positive squamous cell carcinoma (Vul-SSC+).

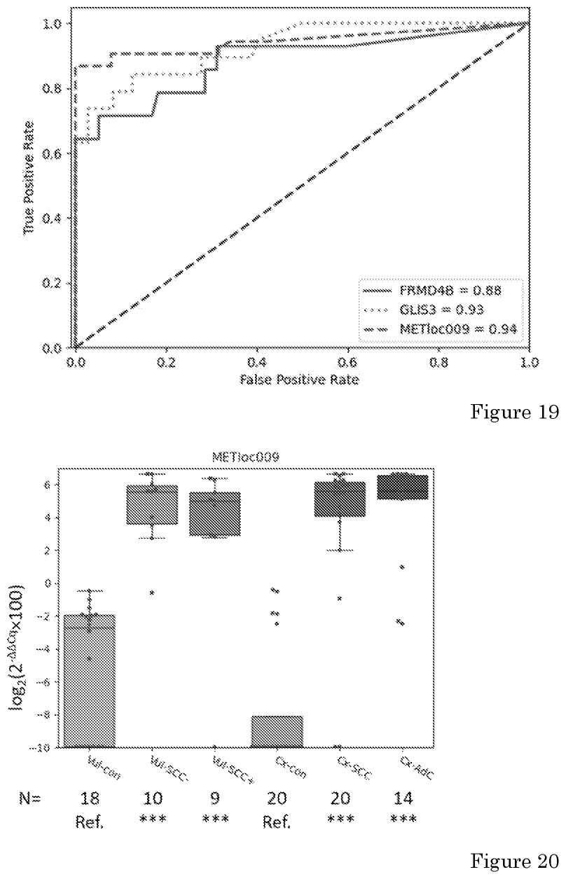

FIG. 19 shows the receiver operating characteristic (ROC) curve analysis and area under the curve (AUC) of methylation ratios for METloc009 (cervical and vulvar cancer), GLIS3 (vulvar cancer) and FRMD4B (cervical adenocarcinoma) in whole tissue sections. Table 9 shows the sensitivity, specificity, positive and negative predictive values, accuracy and Matthews correlation coefficient for the individual markers.

TABLE 9
Classification statistics for METloc009; cervical and vulvar
cancer, GLIS3; vulvar cancer and FRMD4B; cervical adenocarcinoma.
Results for vulvar and cervical cancer in whole tissue
sections. Sensitivity(SEN), specificity(SPC), positive
and negative predictive values(PPV, NPV), accuracy(ACC)
and Matthews correlation coefficient (MCC).

| Marker | SEN | SPC | PPV | NPV | ACC | MCC |
|---|---|---|---|---|---|---|
| METloc009 | 0.87 | 1 | 1 | 0.84 | 0.92 | 0.8527 |
| GLIS3 | 0.84 | 0.88 | 0.64 | 0.95 | 0.87 | 0.6629 |
| FRMD4B | 0.71 | 0.95 | 0.71 | 0.95 | 0.91 | 0.6698 |

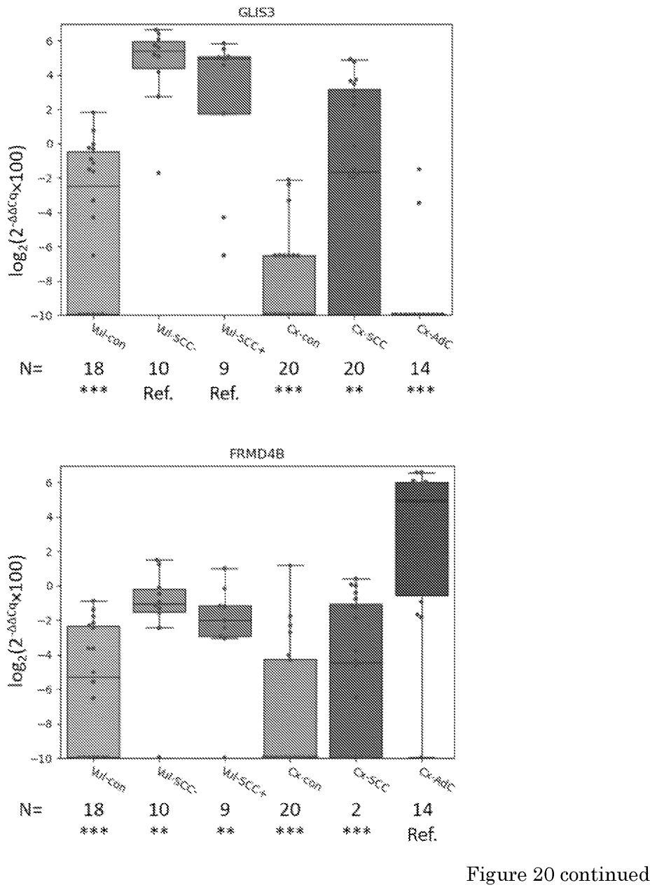

Methylation levels of the markers were calculated for each of the samples and the results are provided in FIG. 20. The methylation levels of METloc009 were clearly increased if vulvar cancer or cervical cancer was present. The methylation levels of GLIS3 was clearly increased if vulvar cancer was present. Finally, the methylation levels of FRMD4B was clearly increased if cervical adenocarcinoma was present.

METloc009 methylation was further determined in colorectal cancer and anal cancer. For this purpose, the methylation levels were analyzed by qMSP as described in MM in test sets of:
18 colorectal formalin fixed paraffin embedded (FFPE) whole tissue samples (WTS); of these 18 samples, 9 were classified as normal colorectal tissue (ColR-con) and 9 as colorectal carcinoma (ColR-car);
13 anal formalin fixed paraffin embedded (FFPE) whole tissue samples (WTS); of these 13 samples, 6 were classified as normal anal (Anal-con) and 7 were classified as anal squamous cell carcinoma (Anal-SCC).

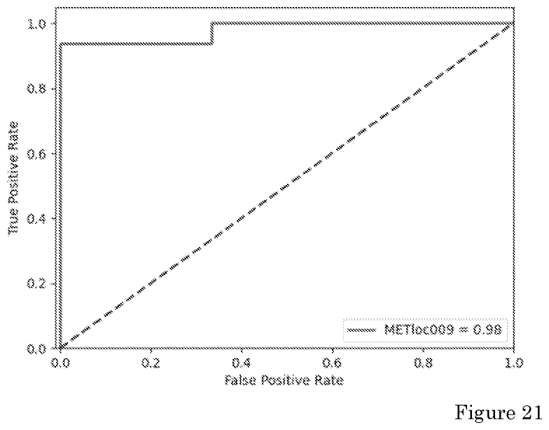

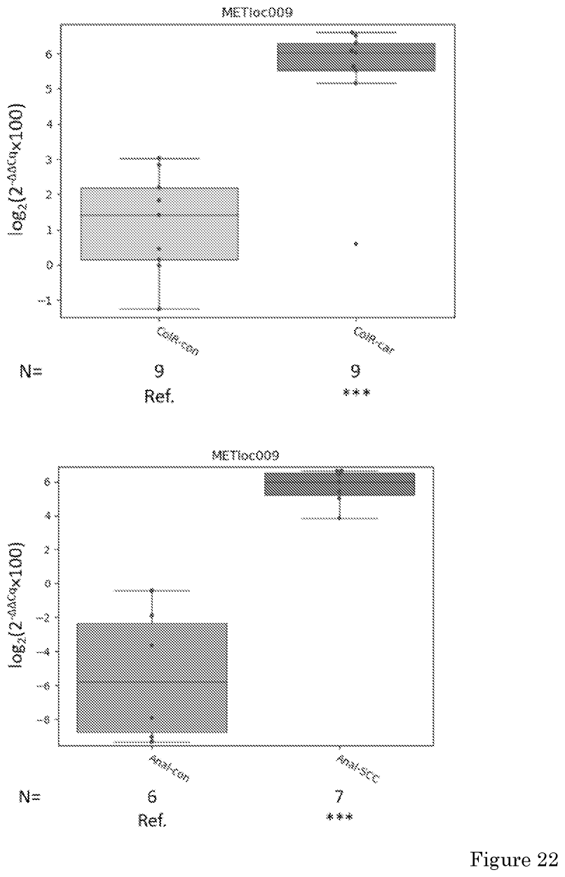

FIG. 21 shows the receiver operating characteristic (ROC) curve analysis and area under the curve (AUC) of methylation ratios for METloc009 in colorectal and anal cancer whole tissue sections. Table 10 shows the sensitivity, specificity, positive and negative predictive values, accuracy and Matthews correlation coefficient for the individual markers.

TABLE 10
Classification statistics for METloc009 detecting colorectal and
anal cancer in whole tissue sections. Sensitivity(SEN), specificity(SPC),
positive and negative predictive values(PPV, NPV), accuracy(ACC)
and Matthews correlation coefficient (MCC).

| Marker | SEN | SPC | PPV | NPV | ACC | MCC |
|---|---|---|---|---|---|---|
| METloc009 | 0.94 | 1 | 1 | 0.94 | 0.97 | 0.9333 |

1.-21. (canceled)

22. A kit of parts comprising means for the detection of DNA methylation of one or more genes and/or promoter regions thereof and/or one or more chromosomal regions selected from table 1, preferably wherein said means comprise one or more primers and/or probes specific for said one or more genes and/or promoter regions thereof and/or one or more chromosomal regions, or to a bisulfite converted sequence thereof.

23. Kit of parts according to claim 22, comprising means for the detection of DNA methylation of two or more genes and/or promoter regions thereof and/or chromosomal regions selected from table 1.

24. Kit of parts according to claim 22, wherein said at least two genes and/or promoter regions thereof and/or chromosomal regions are selected from METloc001, METloc002, ARID3C or promoter region thereof and ARL5C or promoter region thereof.

25.-26. (canceled)

27. A method for determining methylation status of one or more genes and/or promoter regions thereof and/or one or more chromosomal regions selected from table 1 in a sample of an individual.

28. The method according to claim 27, comprising contacting said sample with means for detecting methylation status of said one or more genes and/or promoter regions thereof and/or one or more chromosomal regions selected from table 1.

29. The method according to claim 27, wherein said one or more genes and/or promoter regions thereof and/or one or more chromosomal regions comprise METloc002.

30. The method according to claim 27, wherein said one or more genes and/or promoter regions thereof and/or one or more chromosomal regions comprise METloc001.

31. The method according to claim 27, wherein said one or more genes and/or promoter regions thereof and/or one or more chromosomal regions comprise ARID3C.

32. The method according to claim 27, wherein the methylation status of two or more genes and/or promoter regions thereof and/or chromosomal regions selected from table 1 is determined.

33. The method according to claim 32, wherein the methylation status of at least two genes and/or promoter regions thereof and/or chromosomal regions selected from METloc001, METloc002, ARID3C and ARL5C is determined.

34. The method according to claim 27, wherein said one or more genes and/or promoter regions thereof and/or one or more chromosomal regions comprise METloc001 and METloc002.

35. The method according to claim 27, wherein said one or more genes and/or promoter regions thereof and/or one or more chromosomal regions comprise ARID3C and ARL5C.

36. The method according to claim 27, wherein said sample is a tissue, cell, blood, plasma, serum, saliva, serous fluid, urine or stool sample and/or wherein said sample is a self-sample or a physician-taken sample.

37. The method according to claim 27, wherein said sample is a tissue, liquid biopsy or cell sample.

38. The method according to claim 27, wherein methylation status of said one or more genes and/or promoter regions thereof and/or one or more chromosomal regions selected from table 1 is a multi-cancer marker for a gynaecological, ano-uro-genital, head and neck or cutaneous cancer and precancerous lesions thereof.

39. The method according to claim 27 comprising comparing said DNA methylation with one or more reference values and classifying said individual based on said comparison.

40. The method according to claim 27, comprising classifying the individual as suffering from or being at risk of suffering from a gynaecological, ano-uro-genital, head and neck or cutaneous cancer based on said DNA methylation.

41. The method according to claim 40, wherein said cancer is selected from the group consisting of cervical, vulvar, endometrial, ovarian, fallopian tube, vaginal, colon, colorectal, anal, head and neck, penile, bladder, prostate and cutaneous cancer.

42. The method according to claim 27, wherein said one or more genes and/or promoter regions thereof and/or one or more chromosomal regions selected from table 1 are hypermethylated in at least two, preferably at least three, preferably all, of the following cancers and precancerous lesions: cervical, vulvar, endometrial, fallopian tube, head and neck, cutaneous and ovarian cancer and precancerous lesions thereof.

43. A method of treating an individual suffering from or being at risk of suffering from a gynaecological, ano-uro-genital, head and neck or cutaneous cancer, the method comprising:
classifying an individual as suffering from or being at risk of suffering from a gynaecological, ano-uro-genital, head and neck or cutaneous cancer, by determining DNA methylation of one or more genes and/or promoter regions thereof and/or one or more chromosomal regions selected from table 1 in a sample of said individual, and classifying said individual based on said DNA methylation;
identifying an individual that is suffering from or at risk of suffering from a gynaecological, ano-uro-genital, head and neck or cutaneous cancer; and
referring of the individual to the appropriate medical specialist,
the method further preferably comprising:
identifying the gynaecological, ano-uro-genital, head and neck or cutaneous cancer; and
regular examining of the status and/or monitoring of the cancer or precancerous lesion;
and/or
providing treatment of the cancer or precancerous lesion the identified individual, such as local resection or cryosurgery, laser therapy, hysterectomy, salpingo-oophorectomy, penectomy, transurethral resection, cystectomy, prostatectomy, polypectomy, colectomy, proctectomy, pelvic exenteration, laryngectomy, thyroidectomy, radiation therapy, chemotherapy, hormone therapy, immune therapy and combinations thereof.


SEQUENCE LISTING (24 sequences)

SEQ ID NO: 1 - DNA, 21 bp, synthetic construct
  source 1..21: mol_type = other DNA
  misc_structure 7: note = Locked Nucleic Acid
  misc_structure 8: note = Locked Nucleic Acid
  misc_structure 21: note = Locked Nucleic Acid
tttcggtcgtttttattttac

SEQ ID NO: 2 - DNA, 17 bp, synthetic construct
  source 1..17: mol_type = other DNA
cccaaaccgaacccgta

SEQ ID NO: 3 - DNA, 13 bp, synthetic construct
  source 1..13: mol_type = other DNA
  misc_feature 1..13: note = incorporates a 5 prime fluorescent dye and a minor groove binder (MGB) conjugated to the three prime non fluorescent quencher of the probe
cgcccctaacctc

SEQ ID NO: 4 - DNA, 23 bp, synthetic construct
  source 1..23: mol_type = other DNA
  misc_structure 23: note = Locked Nucleic Acid
gggatttttgtttatcgtatcgc

SEQ ID NO: 5 - DNA, 13 bp, synthetic construct
  source 1..13: mol_type = other DNA
  misc_structure 6: note = Locked Nucleic Acid
tccaccgtcccga

SEQ ID NO: 6 - DNA, 22 bp, synthetic construct
  source 1..22: mol_type = other DNA
  misc_feature 1..22: note = incorporates a 5 prime fluorescent dye and a minor groove binder (MGB) conjugated to the three prime non fluorescent quencher of the probe
aacaaatatcgaaactaaataa

SEQ ID NO: 7 - DNA, 25 bp, synthetic construct
  source 1..25: mol_type = other DNA
tggtgatggaggaggtttagtaagt

SEQ ID NO: 8 - DNA, 27 bp, synthetic construct
  source 1..27: mol_type = other DNA
aaccaataaaacctactcctcccttaa

SEQ ID NO: 9 - DNA, 30 bp, synthetic construct
  source 1..30: mol_type = other DNA
accaccacccaacacacaataacaaacaca

SEQ ID NO: 10 - DNA, 29 bp, synthetic construct
  source 1..29: mol_type = other DNA
gtttgtgttttttttgtttagtcgtattc

SEQ ID NO: 11 - DNA, 23 bp, synthetic construct
  source 1..23: mol_type = other DNA
taataaaaccccgataccgaacg

SEQ ID NO: 12 - DNA, 22 bp, synthetic construct
  source 1..22: mol_type = other DNA
  misc_feature 1..22: note = incorporates a 5 prime fluorescent dye and a minor groove binder (MGB) conjugated to the three prime non fluorescent quencher of the probe
ttttcgttgtttttttcggatt

SEQ ID NO: 13 - DNA, 23 bp, synthetic construct
  source 1..23: mol_type = other DNA
ttgttttttaaagaggtcgggtc

SEQ ID NO: 14 - DNA, 18 bp, synthetic construct
  source 1..18: mol_type = other DNA
ttcctccgtctcgcgaac

SEQ ID NO: 15 - DNA, 21 bp, synthetic construct
  source 1..21: mol_type = other DNA
  misc_feature 1..21: note = incorporates a 5 prime fluorescent dye and a minor groove binder (MGB) conjugated to the three prime non fluorescent quencher of the probe
aattaaaaaaacctctcctcg

SEQ ID NO: 16 - DNA, 21 bp, synthetic construct
  source 1..21: mol_type = other DNA
ttatagaattcgcggcgtagc

SEQ ID NO: 17 - DNA, 18 bp, synthetic construct
  source 1..18: mol_type = other DNA
tcaattcacaaccgaccg

SEQ ID NO: 18 - DNA, 15 bp, synthetic construct
  source 1..15: mol_type = other DNA
  misc_feature 1..15: note = incorporates a 5 prime fluorescent dye and a minor groove binder (MGB) conjugated to the 3 prime non fluorescent quencher of the probe
caaacgaccgctatt

SEQ ID NO: 19 - DNA, 25 bp, synthetic construct
  source 1..25: mol_type = other DNA
agagggagatataagttcggttttc

SEQ ID NO: 20 - DNA, 27 bp, synthetic construct
  source 1..27: mol_type = other DNA
aactacttcctaaatataaacgccgaa

SEQ ID NO: 21 - DNA, 15 bp, synthetic construct
  source 1..15: mol_type = other DNA
  misc_feature 1..15: note = incorporates a 5 prime fluorescent dye and a minor groove binder (MGB) conjugated to the 3 prime non fluorescent quencher of the probe
cgcgagttcgttttt

SEQ ID NO: 22 - DNA, 23 bp, synthetic construct
  source 1..23: mol_type = other DNA
ggagagagggagtcgaaagtttc

SEQ ID NO: 23 - DNA, 21 bp, synthetic construct
  source 1..21: mol_type = other DNA
ttacaactataccccgaaccg

SEQ ID NO: 24 - DNA, 17 bp, synthetic construct
  source 1..17: mol_type = other DNA
  misc_feature 1..17: note = incorporates a 5 prime fluorescent dye and a minor groove binder (MGB) conjugated to the 3 prime non fluorescent quencher of the probe
tccttccgtcctaccta